(19) United States
(12) Patent Application Publication
(10) Pub. No.: US 2024/0140844 A1
(43) Pub. Date: May 2, 2024

(54) WELL TREAMENT COMPOSITIONS INCLUDING ALGAE BIOMASSES AND METHODS FOR MAKING AND USING SAME

(71) Applicant: University Of Louisiana At Lafayette, Lafayette, LA (US)

(72) Inventors: William Chirdon, Lafayette, LA (US); Peter Schexnayder, Lafayette, LA (US); Garrett Thibodeaux, Lafayette, LA (US); Nicholas Baudoin, Lafayette, LA (US)

(73) Assignee: UNIVERSITY OF LOUISIANA AT LAFAYETTE, Lafayette, LA (US)

(21) Appl. No.: 18/207,417

(22) Filed: Jun. 8, 2023

Related U.S. Application Data
(63) Continuation-in-part of application No. 17/012,597, filed on Sep. 4, 2020, now Pat. No. 11,739,253.
(60) Provisional application No. 62/896,628, filed on Sep. 6, 2019.

(51) Int. Cl. C02F 3/32; C12N 1/12

(52) U.S. Cl. CPC C02F 3/325; C12N 1/12; C12R 2001/89

(57) ABSTRACT

Disclosed herein are well treatment composition and methods. In a specific embodiment, a well treatment composition includes: denatured algae, where the denatured algae have a protein content from about 20.0 wt % to about 90.0 wt %, and where the denatured algae have a lipid content from about 0.1 wt % to about 30.0 wt %; one or more carrier fluids; and one or more additives. In another specific embodiment, a method of making a well treatment composition can include contacting one or more algae with one or more denaturants to make a denatured algae, where the denatured algae have a protein content from about 20.0 wt % to about 90.0 wt %, and where the denatured algae have a lipid content from about 0.1 wt % to about 30.0 wt %; and contacting the denatured algae with one or more carrier fluids to make a well treatment composition.

### CROSS REFERENCE TO RELATED APPLICATIONS

This application is a continuation-in-part of U.S. patent application Ser. No. 17/012,597, filed Sep. 4, 2020, which claims the benefit of U.S. Provisional Patent Application No. 62/896,628, filed Sep. 6, 2019, each of which is incorporated by reference herein in its entirety.

### BACKGROUND

### Field

Disclosed herein are well treatment compositions that can be used to treat oil and gas wells.

### Description of the Related Art

The oil and gas industry continuously attempts to develop new technologies that can improve the ability to recover remaining and untapped oil from the subterranean reservoirs. One way that this can be accomplished is through the development of enhanced oil recovery (EOR) technologies. However, with currently available enhanced oil recovery technologies, significant oil reserves are often still left in the reservoirs.

Consequently, there is a need for new well treatment compositions that can be used to treat oil and gas wells to produce untapped subterranean reservoirs.

### SUMMARY

Provided herein are well treatment compositions and methods. In a specific embodiment, a well treatment composition includes: denatured algae, where the denatured algae have a protein content from about 20.0 wt % to about 90.0 wt %, and where the denatured algae have a lipid content from about 0.1 wt % to about 30.0 wt %; one or more carrier fluids; and one or more additives.

In another specific embodiment, A method of making a well treatment composition can include contacting one or more algae with one or more denaturants to make a denatured algae, where the denatured algae have a protein content from about 20.0 wt % to about 90.0 wt %, and where the denatured algae have a lipid content from about 0.1 wt % to about 30.0 wt %; and contacting the denatured algae with one or more carrier fluids to make a well treatment composition.

In another specific embodiment, a method of treating a well or subterranean formation can include: injecting a well treatment composition into a wellbore, where the well treatment composition includes: one or more denatured algae, where the denatured algae have a protein content from about 20.0 wt % to about 90.0 wt %, and where the denatured algae have a lipid content from about 0.1 wt % to about 30.0 wt %; one or more carrier fluids; and one or more additives.

### BRIEF DESCRIPTION OF THE DRAWINGS

The present disclosure can be better understood by referring to the following drawings. The drawings constitute a part of this specification and include exemplary embodiments of the well treatment compositions and methods, which may be embodied in various forms.

FIG. 1 is Rheological data (shear stress plotted vs. shear rate) for sodium hydroxide, which denotes polymer solutions by call codes comprised of three parts: two letters to denote the denaturant, a numerical value indicative of the volume of the denaturant solution in mL and a Greek letter indicating the amount of water added: α for 750 mL, β for 1000 mL, and γ for 1250 mL.

FIG. 2 is Rheological data (shear stress plotted vs. shear rate) for citric acid, which denotes polymer solutions by call codes comprised of three parts: two letters to denote the denaturant, a numerical value indicative of the volume of the denaturant solution in mL and a Greek letter indicating the amount of water added: α for 750 mL, β for 1000 mL, and γ for 1250 mL.

FIG. 3 is Rheological data (shear stress plotted vs. shear rate) for urea, which denotes polymer solutions by call codes comprised of three parts: two letters to denote the denaturant, a numerical value indicative of the volume of the denaturant solution in mL and a Greek letter indicating the amount of water added: α for 750 mL, β for 1000 mL, and γ for 1250 mL.

FIG. 4 is a Heat map of the percent of oil recovery that occurs during polymer flooding relative to the oil in the sand pack before polymer flooding (% RORP) for the denaturants.

FIG. 5a shows viscosity vs. rotational velocity for sodium hydroxide (α).

FIG. 5b shows viscosity vs. rotational velocity for sodium hydroxide (β).

FIG. 5c shows viscosity vs. rotational velocity for sodium hydroxide (γ).

FIG. 5d shows viscosity vs. rotational velocity for citric acid (α).

FIG. 5e shows viscosity vs. rotational velocity for citric acid e (β).

FIG. 5f shows viscosity vs. rotational velocity for citric acid (γ).

FIG. 5g shows viscosity vs. rotational velocity for urea (α≤).

FIG. 5h shows viscosity vs. rotational velocity for urea (β).

FIG. 5i shows viscosity vs. rotational velocity for urea (γ).

FIG. 6 is a graph of Plastic Viscosity (PV) results versus algae and caustic soda concentration.

FIG. 7 is a graph of Yield Point (YP) results versus algae and caustic soda concentration.

FIG. 8 is Fit of H-B parameter τ0 versus algae concentration.

FIG. 9 is Fit of H-B parameter k versus algae concentration.

FIG. 10 is Fit of H-B parameter n versus algae concentration.

FIG. 11 is Fit of H-B parameter τ0 versus caustic concentration.

FIG. 12 is Fit of H-B parameter k versus caustic concentration.

FIG. 13 is Fit of H-B parameter n versus caustic concentration.

FIG. 14 is a graph of Ten second gel strength results versus algae and caustic soda concentration.

FIG. 15 is a graph of Ten-minute gel strength results versus algae and caustic soda concentration.

FIG. 16 is a graph of Thirty-minute gel strength results versus algae and caustic soda concentration.

FIG. 17 is a graph of High-pressure, high temperature filtrate volume versus algae and caustic soda concentration.

FIG. 18 is a graph of Corrected coefficient of friction versus algae and caustic soda concentration.

FIG. 19 is a graph of Coefficient of friction (torque) reduction versus algae and caustic soda concentration.

FIG. 20a is a graph of Plastic Viscosity (PV) change results versus lubricant type.

FIG. 20b is a graph of Plastic Viscosity (PV) change results versus lubricant concentration.

FIG. 21a is a graph of Yield Point (YP) change results versus lubricant type

FIG. 21b is a graph of Yield Point (YP) change results versus concentration.

FIG. 22 is a Fit of H-B parameter τ0 versus algae concentration.

FIG. 23 is a Fit of H-B parameter k versus algae concentration.

FIG. 24 is a Fit of H-B parameter n versus algae concentration.

FIG. 25a is Ten second gel strength change results versus lubricant type.

FIG. 25b is Ten second gel strength change results versus concentration.

FIG. 26a is Ten-minute gel strength change results versus lubricant type.

FIG. 26b is Ten-minute gel strength change results versus concentration.

FIG. 27a is Thirty-minute gel strength change results versus lubricant.

FIG. 27b is Thirty-minute gel strength change results versus concentration.

FIG. 28a is High-pressure, high temperature filtrate volume change versus lubricant type.

FIG. 28b is High-pressure, high temperature filtrate volume change versus concentration.

FIG. 29a is Coefficient of friction (torque) change versus lubricant.

FIG. 29b is Coefficient of friction (torque) change versus concentration.

FIG. 30 is a table showing the viscosity over time for a variety of shear rates. It can be seen that as the RPM of the experiment is increased, the viscosity increases in response to the higher shear rate.

FIG. 31 is a graph showing percent oil produced vs. pH of well treatment compositions from denatured algal biomass.

FIG. 32 is the pH comparison of citric acid mixtures.

FIG. 33 is the pH comparison of urea mixtures.

FIG. 34 is the pH comparison of sodium hydroxide mixtures.

FIG. 35 shows the emulsion produced between free oil and the aqueous phase.

FIG. 36 shows the frequency sweep.

FIG. 37 shows the apparent viscosity as a function of shear rate.

FIG. 38 shows Table 1a: Summary of EOR results for NaOH as denaturant. 70 g of Spirulina powder was added to each water volume. Denaturants with a concentration of 1 M were then added as specified.

FIG. 39 shows Table 1b: Summary of EOR results for citric acid as denaturant. 70 g of Spirulina powder was added to each water volume. Denaturants with a concentration of 1 M were then added as specified.

FIG. 40 shows Table 1c: Summary of EOR results for urea as denaturant. 70 g of Spirulina powder was added to each water volume. Denaturants with a concentration of 1 M were then added as specified.

FIG. 41 shows a chromatogram of Spirulina during treatment with NaOH. The peak at 23 minutes that forms after the added denaturant seems to correlate with better enhancement of rheology including gluing and EOR effects. There are still distinct MW peaks after denaturation, but the peaks have changed. After NaOH addition, there is a strong peak at 23.937 min, which corresponds to approximately 250,000 g/mole molecular weight. I believe this is significant worth claiming. There is a slight increase in the low molecular weight range around 40.4 min (1,100 g/mole) which indicates that most of the protein has not hydrolyzed.

FIG. 42 shows a chromatogram of Chlorella during treatment with NaOH.

Denaturing causes the high MW peak at 14.823 min (5,073,000 g/mole) to decrease in amount and in molecular weight to 15.038 min (4,726,000 g/mole).

FIG. 43 shows a work flowchart for the performance mechanisms of Spirulina-based EOR Fluids.

FIG. 44 shows a flowchart for the well treatment composition.

FIG. 45 shows a table EOR, pH, phase, and interfacial data for well treatment compositions and baseline fluids.

FIGS. 46A, 46B and 46C show an amplitude sweep for well treatment compositions, between the range of 0.1-1000%, with 0.33%, 1.33%, and 2.33% concentrations of 1 M denaturants at 20° C.

FIGS. 47A, 47B and 47C shows a frequency sweep for well treatment compositions, between the range of 0.1-100 rads·^{s-1}, with 0.33%, 1.33%, and 2.33% concentrations of 1 M denaturant at 20° C.

FIGS. 48A, 48B, 48C, 48D, 48E and 48F show the flow curve of shear stress as a function of shear rate within (FIGS. 48A, 48C and 48E) the range of 0-20,000 s⁻¹, and (FIGS. 48B, 48D and 48F) the range of 0-200 s⁻¹ for well treatment compositions with 0.33%, 1.33%, and 2.33% concentration of 1 M denaturant at 20° C.

FIGS. 49A, 49B, 49C, 49D, 49E and 49F show the flow curve of viscosity as a function of shear rate within (FIGS. 49A, 49C and 49E) the range of 0-20,000 s⁻¹, and (FIGS. 49B, 49D and 49F) the range of 0-1,000 s⁻¹ for well treatment compositions with 0.33%, 1.33%, and 2.33% concentration of 1 M denaturant at 20° C.

FIGS. 50A, 50B, 50C and 50D show visual and intuitive representation of the high denaturant concentration effects on the “supernatant” and “settled” phases of the well treatment compositions that were observed after centrifugation. Depicted above are the undenatured EOR Fluid (50A), citric acid-based EOR fluid (50B), urea-based EOR fluid (50C), and sodium hydroxide-based EOR fluid (50D).

FIGS. 51A, 51B and 51C show a 3-Interval-Thixotropy Test for well treatment compositions (51A, 51B, 51C) with 0.33%, 1.33%, and 2.33% of 1 M denaturant at 20° C.

FIG. 52 shows a Table 6 of properties of the well treatment compositions.

### DETAILED DESCRIPTION

Proteinaceous enhanced oil recovery operates on the principle that proteins can be denatured using chemicals to break them down into a viscous polymeric mixture that exhibits an EOR effect. In one or more embodiments, the proteins in question are sourced from an algae (Spirulina) meal, which could be obtained as a waste product from wastewater treatment or bioprocessing operations, including biofuel production where the lipids are extracted for fuel but the large volumes of proteins are considered a waste material with little or negative value. In other embodiments, other biomasses are used, especially edible biomasses, thereby mitigating public resistance to drilling operations due to concerns of perceived toxicity of chemicals. This also mitigates legal liability from the use of chemicals that are known to be toxic. Additionally, the agents are denatured biomasses that have not undergone expensive purification or separation steps. This will make these agents highly cost competitive. And, because there is no separation or purification, there are no wastes or byproducts from the synthesis to be disposed of This also reduces costs compared to competitors.

In one or more embodiments, the well treatment compositions can include, but are not limited: one or more algae, one or more carrier fluids and/or solvents, and one or more additives. The well treatment compositions can provide a method for enhancing oil production which, although a type of chemical flooding (i.e., the injection of chemicals suspended in water in order to produce oil), does not emulsify the recovered oil as much as conventional EOR agents. Conventional EOR agents use emulsification as a primary drive mechanism and may emulsify nearly all the oil. In contrast, the well treatment compositions produce a smaller volume of emulsion. Without wanting to bound by theory, the well treatment the EOR drive mechanism is assumed to be related to the complex rheology and interfacial effects of the fluid.

The well treatment composition can have increased effectiveness, along with the benefits of renewable, sustainable biomass. The well treatment composition can be made from extremely inexpensive waste proteins that have little or no value, and in some instances have negative value. Thus, a company could be paid for removing waste proteins that would otherwise need to be landfilled, producing a collateral environmental benefit. Further, water streams that are polluted with organics/nutrients could be treated with microbes which could then be used to create these agents, thereby cleaning up polluted water streams, which is also a valuable service that can be monetized. In fact, microbes could also be harvested from polluted waterways where algal/microbial overgrowth has already occurred.

The well treatment composition can include wide variety of algae. The biomass can include, but is not limited to: living algae, dead algae or mixtures thereof. The one or more algae can include, but is not limited to: microalgae. The one or more algae can include, but are not limited to: non-oleaginous microalgae. The one or more algae can include, but are not limited to: algae from the phylum, cyanobacteria, chlorophyta, and mixtures thereof. The one or more algae can include, but are not limited to: algae from the class cyanophyceae, trebouxiophyceae, and mixtures thereof. The one or more algae can include, but are not limited to: algae from the order, spirulinales, chlorellales, and mixtures thereof. The one or more algae can include, but are not limited to: algae from the family, Spirulinaceae, Chlorellaceae, and mixtures thereof. The one or more algae can include, but are not limited to: algae from the Genus, Spirulina. In an embodiment, the Spirulina plantesis biomass can include about 45 vol % crude protein, above the average (42.8 vol. %) for algae used in biofuels, and both the grinding and the drying method used causes ruptures to cell walls, exposing more protein to the denaturants. In an embodiment, the algae biomass can include proteinaceous biomass with a low oil content. In an embodiment, the proteinaceous biomass can include unrefined proteinaceous biomass. In an embodiment, the one or more algae can include, but are not limited to: waste algae biomass. This waste product is becoming increasingly prevalent from biofuel sources because algae possesses both a higher oil content and has a higher carbon capture ability than most biofuel feedstocks and produces what is considered a carbon-neutral fuel.

The algae biomasses can include waste or byproduct of some sort of bioprocess. By creating a large-volume market for these waste biomasses, it can make the other bioprocesses viable or more profitable. For example, it could make biofuels from algae oil viable, or it could be used to recover costs related to wastewater treatment operations, including but not limited to municipal, agricultural, and industrial wastewater cleanup operations. Also, these biomasses could be resulting from wastewater cleanup, that is using an industrial bioprocess to clean up an organically polluted stream such as farm run-off. They also could result from environmental cleanup of lakes or waterways that were polluted by an algal bloom.

This well treatment composition covers agents for enhanced oil recovery (EOR) and additives for drilling fluids derived from proteinaceous biomasses. In the definition of biomass, we claim biomasses that are proteinaceous with at least 10 dry wt % of protein before or after modification with denaturant. In another embodiments, biomasses that are high in protein or mostly protein (over 50 dry wt %) may be used. In another embodiments, the biomasses can include, but is not limited to: one or more microbes. In another embodiments, the biomasses can include, but is not limited to: autotrophs, heterotrophs, and mixotrophs.

In an embodiment, the biomasses of microalgae or derived from microalgae with protein contents of at least 10 dry wt %. In other embodiments we claim biomasses of microalgae or derived from microalgae with protein contents of over 50 dry wt %. Of particular interest are “waste” proteins which have little value or even negative value. A significant aspect of the novelty of the well treatment composition is that biomasses, including unrefined biomasses, can be used to create agents without the need for expensive separations or purifications, thereby making this technology highly cost-competitive in comparison to others.

The one or more well treatment compositions can have a content of the one or more algae the varies widely. For example, the well treatment compositions can have a content of the one or more algae from a low of about 0.1 wt. %, about 1.0 wt. %, or about 5.0 wt. %, to a high of about 90.0 wt. %, about 95.0 wt. %, or about 99.9 wt. %. In another example, the well treatment compositions can have a content of the one or more algae from about 0.1 wt. % to about 99.9 wt. %, about 1.0 wt. % to about 99.0 wt. %, about 0.1 wt. % to about 9.0 wt. %, about 10.0 wt. % to about 90.0 wt. %, about 10.0 wt. % to about 20.0 wt. %, about 20.0 wt. % to about 30.0 wt. %, about 25.0 wt. % to about 75.0 wt. %, about 20.0 wt. % to about 80.0 wt. %, about 20.0 wt. % to about 30.0 wt. %, about 20.0 wt. % to about 60.0 wt. %, about 30.0 wt. % to about 40.0 wt. %, about 30.0 wt. % to about 70.0 wt. %, about 40.0 wt. % to about 60.0 wt. %, about 45.0 wt. % to about 55.0 wt. %, about 40.0 wt. % to about 50.0 wt. %, about 69.0 wt. % to about 75.0 wt. %, about 68.0 wt. % to about 82.0 wt. %, about 72.0 wt. % to about 86.0 wt. %, about 50.0 wt. % to about 73.0 wt. %, about 33.0 wt. % to about 48.0 wt. %, about 60.0 wt. % to about 70.0 wt. %, about 71.0 wt. % to about 81.0 wt. %, about 20.0 wt. % to 30.0 wt. %, about 50.0 wt. % to about 60.0 wt. %, or about 70.0 wt. % to about 80.0 wt. %. The weight percent of the one or more algae in the well treatment compositions can be based on the total weight of the well treatment compositions, or based on the total weight of the one or more algae, one or more carrier fluids, and one or more additives. In another example, the well treatment composition can have an algae from about 10 lb/bbl to about 40 lb/bbl.

The one or more algae can have a molecular weight that varies widely. For example, the one or more algae can have a molecular weight from a low of about 10,000 g/mol, about 100,000 g/mol, or about 150,000 g/mol, to a high of about 300,000 g/mol, about 400,000 g/mol, or about 500,000 g/mol. In another example, the one or more polymers can have a weight-average molecular weight that is less than 300,000 g/mol, less than 250,000 g/mol, or less than 200,000 g/mol. In another example, the one or more polymers can have a weight-average molecular weight from about 10,000 g/mol to about 500,000 g/mol, about 50,000 g/mol to about 275,000 g/mol, about 100,000 g/mol to about 300,000 g/mol, about 200,000 g/mol to about 250,000 g/mol, about 200,000 g/mol to about 275,000 g/mol.

The denatured algae can have a dry protein content that varies widely. For example, the denatured algae can have a dry protein content from a low of about 1.0 wt %, about 5.0 wt %, or about 10.0 wt %, to a high of about 90.0 wt %, about 95.0 wt %, or about 99.0 wt %. In another example, the denatured algae can have a dry protein content from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 20.0 wt % to about 90.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 50.0 wt % to about 70.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. In another example, the denatured algae can have a dry protein content of greater than 30 wt %, greater than 50%, or greater than 60%.

The denatured algae can have a dry lipid content that varies widely. For example, the denatured algae can have a dry lipid content from a low of about 1.0 wt %, about 5.0 wt %, or about 10.0 wt %, to a high of about 90.0 wt %, about 95.0 wt %, or about 99.0 wt %. In another example, the denatured algae can have a dry lipid content from about 0.1 wt % to about 99.9 wt %, about 1.0 wt % to about 99.0 wt %, about 1.0 wt % to about 30.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. In yet another example, the denatured algae can have a dry lipid content of less than 30% less than 20% or less than 10% lipid content.

The one or more solvents and/or carrier fluids can include, but are not limited to: water, hexanes, toluene, methanol, ethanol, propanol, isopropanol, acetone, acetonitrile, chloroform, diethyl ether, methylene chloride, dimethyl formamide, ethylene glycol, propylene glycol, triethylamine, tetrahydrofuran, and mixtures thereof.

The one or more well treatment compositions can have a content of the one or more carrier fluids and/or solvents the varies widely. For example, the well treatment compositions can have a content of the one or more the one or more carrier fluids and/or solvents from a low of about 0.1 wt. %, about 1.0 wt. %, or about 5.0 wt. %, to a high of about 90.0 wt. %, about 95.0 wt. %, or about 99.9 wt. %. In another example, the well treatment compositions can have a content of the one or more the one or more carrier fluids and/or solvents from about 0.1 wt. % to about 99.9 wt. %, about 1.0 wt. % to about 99.0 wt. %, about 10.0 wt. % to about 90.0 wt. %, about 10.0 wt. % to about 20.0 wt. %, about 20.0 wt. % to about 30.0 wt. %, about 25.0 wt. % to about 75.0 wt. %, about 20.0 wt. % to about 80.0 wt. %, about 20.0 wt. % to about 30.0 wt. %, about 20.0 wt. % to about 60.0 wt. %, about 30.0 wt. % to about 40.0 wt. %, about 30.0 wt. % to about 70.0 wt. %, about 40.0 wt. % to about 60.0 wt. %, about 45.0 wt. % to about 55.0 wt. %, about 40.0 wt. % to about 50.0 wt. %, about 69.0 wt. % to about 75.0 wt. %, about 68.0 wt. % to about 82.0 wt. %, about 72.0 wt. % to about 86.0 wt. %, about 50.0 wt. % to about 73.0 wt. %, about 33.0 wt. % to about 48.0 wt. %, about 60.0 wt. % to about 70.0 wt. %, about 71.0 wt. % to about 81.0 wt. %, about 20.0 wt. % to 30.0 wt. %, about 50.0 wt. % to about 60.0 wt. %, or about 70.0 wt. % to about 80.0 wt. %. The weight percent of the one or more algae in the well treatment compositions can be based on the total weight of the well treatment compositions, or based on the total weight of the one or more algae, one or more carrier fluids, and one or more additives.

The one or more additives can include, but are not limited to: one or more acids, one or more bases, one or more salts, one or more polymers, one or more weighting materials, one or more viscosifiers, one or more thinners, one or more dispersants, one or more temperature stability agents, one or more flocculants, one or more filtrate reducers, one or more pH control additives, one or more lost circulation materials, one or more lubricants, one or more shale control materials, one or more emulsifiers, one or more surfactants, one or more bactericides, one or more pipe-freeing agents, one or more corrosion inhibitors, one or more scale inhibitors, one or more breakers, one or more proppants, one or more friction reducers, one or more solvents and/or carrier fluid, and mixtures thereof.

The one or more salts can include, but are not limited to: cesium formate (HCOOCs), sodium chloride (NaCl), sodium carbonate (Na₂CO₃), sodium bicarbonate (NaHCO₃), potassium chloride (KCl), potassium carbonate (K₂CO₃), potassium bicarbonate (KHCO₃), potassium fluoride (KF), sodium fluoride (NaF), potassium formate (HCOOK), sodium formate (HCOONa), calcium chloride (CaCl₂), ammonium carbonate ((NH₄)₂CO₃), ammonium chloride (NH₄C₁), tetramethylammonium chloride (N(CH₃)₄Cl), sodium chloride (NaCl), potassium chloride (KCl), dipotassium glutarate (C₅H₆K₂O₄), disodium glutarate (C₅H₆K₂O₄), sodium citrate (Na₃C₆H₅O₇), potassium citrate (K₃C₆H₅O₇), potassium acetate (CH₃CO₂K), choline chloride [((CH₃)₃NCH₂CH₂OH)Cl], sodium acetate (CH₃CO₂Na), and mixtures thereof.

The one or more bases can include, but are not limited to: calcium hydroxide [Ca(OH)₂], sodium hydroxide (NaOH), potassium hydroxide (KOH), and mixtures thereof.

The one or more acids can include, but are not limited to: hydrochloric acid (HCl), carbonic acid (H₂CO₃), formic acid (CH₂O₂), citric acid (C₆H₈O₇), and mixtures thereof.

The one or more polymers can include, but are not limited: one or more celluloses, one or more carboxymethyl celluloses, one or more polypropylene glycol, one or more polyethylene glycol, one or more polyacrylamide, and mixtures thereof.

The one or more polymers can have a weight-average molecular weight (M_{w}) that varies widely. For example, the one or more polymers can have a weight-average molecular weight from a low of about 300 g/mol, about 3,000 g/mol, or about 10,000 g/mol, to a high of about 80,000 g/mol, about 100,000 g/mol, or about 200,000 g/mol. In another example, the one or more polymers can have a weight-average molecular weight that is less than 8,000 g/mol, less than 5,000 g/mol, or less than 1,000 g/mol. In another example, the one or more polymers can have a weight-average molecular weight from about 300 g/mol to about 200,000 g/mol, about 300 g/mol to about 1,200 g/mol, about 1,000 g/mol to about 10,000 g/mol, about 2,000 g/mol to about 50,000 g/mol, about 100,000 g/mol to about 200,000 g/mol.

The one or more polymers can have a number-average molecular weight (Mₙ) that varies widely. For example, the one or more polymers can have a number-average molecular weight from a low of about 300 g/mol, about 3,000 g/mol, or about 4,000 g/mol, to a high of about 80,000 g/mol, about 100,000 g/mol, or about 200,000 g/mol. In another example, the one or more polymers can have a number-average molecular weight that is less than 1,000 g/mol, less than 6,000 g/mol, or less than 10,000 g/mol. In another example, the one or more polymers can have a number-average molecular weight from about 300 g/mol to about 25,000 g/mol, about 30,000 g/mol to about 200,000 g/mol, about 20,000 g/mol to about 80,000 g/mol, about 40,000 g/mol to about 80,000 g/mol, about 100,000 g/mol to about 200,000 g/mol. The molecular weight of the one or more polymers can be measured by gel permeation chromatography with tri-detectors.

The one or more well treatment compositions can have a content of the one or more additives the varies widely. For example, the well treatment compositions can have a content of the one or more additives from a low of about 0.1 wt. %, about 1.0 wt. %, or about 5.0 wt. %, to a high of about 90.0 wt. %, about 95.0 wt. %, or about 99.9 wt. %. In another example, the well treatment compositions can have a content of the one or more additives from about 0.1 wt. % to about 99.9 wt. %, about 1.0 wt. % to about 99.0 wt. %, about 10.0 wt. % to about 90.0 wt. %, about 10.0 wt. % to about 20.0 wt. %, about 20.0 wt. % to about 30.0 wt. %, about 25.0 wt. % to about 75.0 wt. %, about 20.0 wt. % to about 80.0 wt. %, about 20.0 wt. % to about 30.0 wt. %, about 20.0 wt. % to about 60.0 wt. %, about 30.0 wt. % to about 40.0 wt. %, about 30.0 wt. % to about 70.0 wt. %, about 40.0 wt. % to about 60.0 wt. %, about 45.0 wt. % to about 55.0 wt. %, about 40.0 wt. % to about 50.0 wt. %, about 69.0 wt. % to about 75.0 wt. %, about 68.0 wt. % to about 82.0 wt. %, about 72.0 wt. % to about 86.0 wt. %, about 50.0 wt. % to about 73.0 wt. %, about 33.0 wt. % to about 48.0 wt. %, about 60.0 wt. % to about 70.0 wt. %, about 71.0 wt. % to about 81.0 wt. %, about 20.0 wt. % to 30.0 wt. %, about 50.0 wt. % to about 60.0 wt. %, or about 70.0 wt. % to about 80.0 wt. %. In another example, the well treatment compositions can be free of the one or more additives. The weight percent of the one or more additives in the well treatment compositions can be based on the total weight of the well treatment compositions, or based on the total weight of the one or more inorganic salts, one or more organic salts, one or more acids, one or more bases, one or more polymers, one or more solvents and/or carrier fluids, and one or more additives.

The well treatment compositions can have a content of NaOH and/or urea and/or citric acid that varies widely. For example, the content well treatment compositions o NaOH and/or urea and/or citric acid from about 0.25 to about 1.0 lb barrel. After NaOH addition, there is a strong peak at 23.937 min, which corresponds to approximately 250,000 g/mole molecular weight. There is a slight increase in the low molecular weight range around 40.4 min (1,100 g/mole) which indicates that most of the protein has not hydrolyze. Denaturing causes the high MW peak at 15.038 min (4,726,000 g/mole) to increase in amount and in molecular weight to 14.823 min (5,073,000 g/mole). This is novel and non-obvious because alkaline denaturation is thought to result in hydrolysis yielding more, smaller protein fragments. In this case, a buildup of more larger MW structures is observed in denaturation.

The well treatment composition can have a solids content that varies widely. For example, the well treatment composition can have solids content from a low of about 0.1 wt %, about 10.0 wt %, or about 20.0 wt %, to a high of about 90.0 wt %, about 95.0 wt %, or about 99.9 wt %. In other example, the well treatment composition can have a solids content from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the solids content in the well treatment composition can be based on the total weight of the well treatment compositions, or based on the one or more algae, the one or more additives. As used herein, the solids content of the well treatment composition, as understood by those skilled in the art, can be measured by determining the weight loss upon heating a small sample, e.g., 1-5 grams of the resin, to a suitable temperature, e.g., 125° C., and a time sufficient to remove the liquid therefrom.

The well treatment composition can have a viscosity that varies widely. For example, the well treatment composition can have a viscosity from a low of about 1 cP, about 100 cP, or about 10,000 cP, to a high of about 250,000 cP, about 900,000 cP, or about 2,500,000 cP. In another example, the well treatment composition can have a viscosity from about 1 cP to about 2,500,000 cP, about 10 cP to about 1,000 cP, about 10 cP to about 250,000 cP, about 2,500 cP to about 250,000 cP, about 2,500 cP to about 200,000 cP, about 10,000 cP to about 100,000 cP, about 10,000 cP to about 50,000 cP, about 100,000 cP to about 250,000 cP, about 620,000 cP to about 850,000 cP, about 700,000 cP to about 750,000 cP, about 700,000 cP to about 800,000 cP, about 650,000 cP to about 855,000 cP, about 700,000 cP to about 800,000 cP, about 500,000 cP to about 1,000,000 cP, or about 500,000 cP to about 2,500,000 cP. The viscosity of the well treatment composition can be measured on a Brookfield viscosimeter. The viscosity of the well treatment compositions can be measured at various temperatures, such as 25° C., 40° C., 60° C., and 100° C.

The well treatment composition can have viscosities over shear rate that varies widely. For example, the well treatment composition can have a viscosity from about 0.005 to about 0.015 Pa·s over the shear rate range of about 0 to about 20000 s⁻¹. In another example, the well treatment composition can include a shear thinning range at shear rates approximately less than 2000 s⁻¹, have a shear thickening range roughly in the 2000-5000 s⁻¹ range, then resume shear thinning above 5000 s⁻¹, approximately.

The well treatment composition can include a plastic viscosity that varies widely. For example, the well treatment composition can include a plastic viscosity from a low of about 1.0 cP, about 3.0 cP, or about 5.0 cP, to a high of about 40.0 cP, about 60.0 cP, or about 200.0 cP. In another example, the well treatment composition can include a plastic viscosity from about 1.0 cP to about 200.0 cP, about 2.0 cP to about 10.0 cP, about 5.0 cP to about 40.0 cP or about 7.0 cP to about 60 cP. The plastic viscosity can be based on Bingham plastic model and industry standards.

The well treatment composition can include a yield point that varies widely. For example, the well treatment composition can include yield point from a low of about 3.0 lbf/100 ft², about 5.0 lbf/100 ft², about 8.0 lbf/100 ft², to a high of about 40 lbf/100 ft², about 50 lbf/100 ft², or about 70 lbf/100 ft².

The well treatment composition can include a consistency index (k) within the range of 0.24-1.20 lbf-sⁿ/100 ft²), and power-low index (n) within the range of 0.60 and 0.70, and a yield stress ranging between 0.7 and 15 lbf/100 ft², based on the Hershel-Bulkley model.

The well treatment composition can have a gel strength that varies widely. For example, the well treatment composition can have a 10 s gel strength from about 1 lbf/100 ft² to about 30 lbf/100 ft². In another example, the well treatment composition can have a 10 m gel strength from about 1 lbf/100 ft² to about 50 lbf/100 ft². In another example, the well treatment composition can have a 30 m gel strength from about 1 lbf/100 ft² to about 75 lbf/100 ft².

The pH of the well treatment composition can have a pH vary widely. For example, the well treatment composition can have a pH from a low of about 0.1, about 1.0, about 2.0, to a high of about 12.0, about 13.0 or about 14.0. In another example, the well treatment composition can have a pH from about 0.1 to about 13.9, about 4.0 to about 12.0, about 5.0 to about 8.0, about 5.0 to about 10.0, about 6.0 to about 7.0, about 6.0 to about 8.0, about 7.5 to about 11.0, about 7.0 to about 10.0, about 8.0 to about 9.0, about 9.0 to about 10.0, about 8.0 to about 10.0, about 9.0 to about 11.0, or about 6.0 to about 9.0.

The well treatment composition can have a fluid loss as assessed by filtrate volumes that varies widely. For example, the well treatment composition can have fluid loss as assessed by filtrate volumes ranging from about 12 to about 60 mL.

The well treatment composition can have corrected coefficient of friction that varies widely. For example, the well treatment composition can have a corrected coefficient of friction from about 0.15 to about 0.30.

The well treatment composition can have coefficient of friction reduction that varies widely. For example, the well treatment composition can have coefficient of friction reduction from about 15% to about 37%.

The well treatment composition can have a surface tension that varies widely. For example, the well treatment composition can have a surface tension from a low of about 1 mN/m, about 5 mN/m, about 15 mN/m, to high of about 30 mN/m, about 40 mN/m, about 50 mN/m. For example, the well treatment composition can have a surface tension from about 1 mN/m to about 50 mN/m, about 5 mN/m to about 25 mN/m, about 10 mN/m to about 40 mN/m, or about 20 mN/m to about 45 mN/m.

The well treatment composition can have an interfacial tension that varies widely. For example, the well treatment composition can have an interfacial tension from a low of about 0.1 mN/m, about 0.5 mN/m, about 0.75 mN/m, to high of about 1.0 mN/m, about 1.2 mN/m, about 2.1 mN/m. For example, the well treatment composition can have a interfacial tension from about 0.1 mN/m to about 2.1 mN/m, about 0.5 mN/m to about 1.5 mN/m, about 0.9 mN/m to about 1.7 mN/m, or about 1.0 mN/m to about 1.2 mN/m

The well treatment composition can have a contact angle on quartz in air that varies widely. For example, the well treatment composition can have a contact angle on quartz in air from about 1° to about 50°. In another example, the well treatment composition can have a contact angle on quartz in air of less than 50°.

The well treatment composition can have a storage modulus (G′) greater than the loss modulus (G″) with a crossover point greater than 1% strain when tested in a frequency-controlled strain sweep at a frequency of 10 rad*s⁻¹. For example, the well treatment composition can have a modulus having a G′ and G″ ranging between 0.1 and 10 Pa at strains less than 1%.

The well treatment composition can be stable at low shear intervals of 10 s⁻¹. The well treatment composition can have break down of structure at 200 s⁻¹.

In or more embodiments, the methods of making the well treatment composition can include, but are not limited to: obtaining algae biomass, extracting at least a portion of algae biomass, grinding the at least portion of the algae biomass, contacting the grinded the algae biomass with one or more solvents to produce one or more mixtures, contacting the one or more mixtures with a denaturant to make a denatured algae biomass. In another embodiment, the method of making the well treatment composition can include, but is not limited to: contacting one or more aglae biomasses with one or more denaturants in one or more mixtures to make a denatured algae biomass. For example, the one or more mixtures can include, but are not limited to: a first mixtures, second mixtures, third mixture, fourth mixture, and more mixtures.

The algae can be denatured by the one or more acids and the one or more bases. These denaturants cause folded proteins to break their secondary bonds, reverting them from complex, folded quaternary structures to randomly coiled primary structures which entangle with one another to create a viscous fluid, which is then diluted in water to reduce viscosity to levels manageable for oilfield pumps.

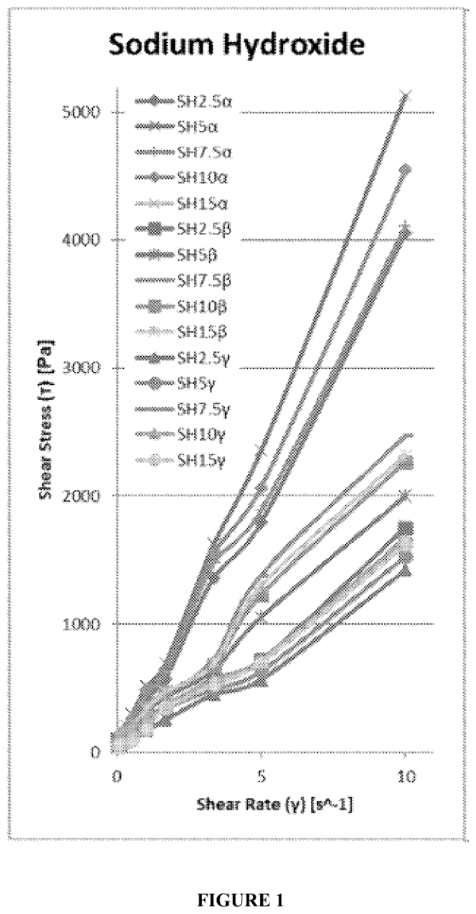

The denaturants can include, but are not limited: the one or more acids and the one or more bases. FIGS. 1, 2, and 3, shows the rheological data for the denaturants of sodium hydroxide, citric acid, and urea. After NaOH addition, there is a strong peak at 23.937 min, which corresponds to approximately 250,000 g/mole molecular weight. However, other denaturants may be used. All three of these preferred denaturants are waste products or byproducts of various industries. These inexpensive chemicals are only needed in small concentrations relative to the volume of EOR fluid they generate.

The pH of the first mixture, second mixture, third mixture, fourth mixture and more mixtures can have a pH vary widely. For example, the first mixture, second mixture, third mixture, fourth mixture and more mixtures can have a pH from a low of about 0.1, about 1.0, about 2.0, to a high of about 12.0, about 13.0 or about 14.0. In another example, the first mixture, second mixture, third mixture, fourth mixture and more mixtures can have a pH from about 0.1 to about 13.9, about 4.0 to about 12.0, about 5.0 to about 10.0, about 7.5 to about 11.0, about 7.0 to about 10.0, about 8.0 to about 9.0, about 9.0 to about 10.0, about 8.0 to about 10.0, about 9.0 to about 11.0, or about 6.0 to about 9.0.

The first mixture, first reaction mixture, second mixture, second reaction mixture, third mixture, third reaction mixture, fourth mixtures or more mixtures can have a viscosity that varies widely. For example, the first mixture, first reaction mixture, second mixture, second reaction mixture, third mixture, third reaction mixture, fourth mixtures or more mixtures can have a viscosity from a low of about 1 cP, about 100 cP, or about 100 cP, to a high of about 5,000 cP, to about 9,000 cP, or about 10,000 cP. In another example, the first mixture, first reaction mixture, second mixture, second reaction mixture, third mixture, third reaction mixture, fourth mixtures or more mixtures can have a viscosity from about 1 cP to about 10 cP, about 1 cP to about 500 cP, about 10 cP to about 50 cP, about 10 cP to about 100 cP, about 100 cP to about 500 cP, about 6,200 cP to about 8,500 cP, about 7,387 cP to about 7,500 cP, about 7,000 cP to about 8,000 cP, about 6,500 cP to about 8,550 cP, about 7,000 cP to about 8,000 cP, or about 5,000 cP to about 10,000 cP.

The first mixture, first reaction mixture, second mixture, second reaction mixture, third mixture, third reaction mixture, fourth mixtures or more mixtures can be heated to a temperature from a low of about 0° C., about 15° C., and about 25° C., to a high of about 35° C., about 65° C., and about 200° C. For example, the first mixture, first reaction mixture, second mixture, second reaction mixture, third mixture, third reaction mixture, fourth mixtures or more mixtures can be heated to a temperature from about 25° C. to about 28° C., about 25° C. to about 35° C., about 25° C. to about 90° C., about 30° C. to about 45° C., about 40° C. to about 90° C., about 43° C. to about 78° C., about 40° C. to about 90° C., about 100° C. to about 200° C. In another example, the first mixture, first reaction mixture, second mixture, second reaction mixture, third mixture, third reaction mixture, fourth mixtures or more mixtures can be at room temperature.

The first mixture, first reaction mixture, second mixture, second reaction mixture, third mixture, third reaction mixture, fourth mixtures or more mixtures can be reacted and/or stirred for a first reaction time, second reaction time, third reaction time, or more reaction times from a short of about 15 s, about 120 s, or about 300 s, to a long of about 1 h, about 24 h, or about 72 h. For example, first mixture, first reaction mixture, second mixture, second reaction mixture, third mixture, third reaction mixture, fourth mixtures or more mixtures can be from about 1 min to about 15 min, about 5 min to about 45 min, about 1 h to about 12 h, about 5 h to about 15 h, about 10 hours to about 24 hours, about 12 h to about 17 h, about 12 h to about 24 h, about 22 h to about 50 h, or about 24 h to about 72 h.

The algae can have an elution time that varies widely. For example, the algae can have an elution time from a short of about 15 s, about 120 s, or about 300 s, to a long of about 1 h, about 24 h, or about 72 h. In another example, algae can have an elution time from about 1 min to about 15 min, about 5 min to about 45 min, about 1 h to about 12 h, about 5 h to about 15 h, about 10 hours to about 24 hours, about 12 h to about 17 h, about 12 h to about 24 h, about 22 h to about 50 h, or about 24 h to about 72 h. Chromatography can be based on size-exclusion. As such, translating the elution time to molecular weight requires some assumptions based on the calibration standards. In short, one concern is that the denaturation might not be making the macromolecules more massive terms of weight, but instead just makes them larger which then appears to be higher molecular weight since this is not accounted for in the simple calibration which does not account for changes in conformation.

Without wanting to be bound by theory, it could be that the alkaline denaturation it often thought to result in hydrolysis yielding more, smaller protein fragments. Hence, a buildup of more larger molecular weight structures may be occurring. The chromatography based on size-exclusion. As such, translating the elution time to molecular weight requires some assumptions based on the calibration standards. The denaturation might not be making the macromolecules more massive terms of weight, but instead just makes them larger which then appears to be higher MW since this is not accounted for in the calibration.

The well treatment compositions can be used in well drilling fluids, well completion fluids, and well treatment compositions. The well treatment compositions can be used in oil-based well treatment compositions, synthetic-based well treatment compositions, and water-based well treatment compositions. In one or more embodiments, the method of using the well treatment compositions can include, but are not limited to: injecting a well treatment composition down a wellbore.

Tertiary EOR using polymer flooding is an established technology in the art using synthetic polymer, and some variations using polymers produced by microbes have been proposed. The current well treatment composition differs in that it utilizes a non-living, whole biomass that is proteinaceous.

Since the EOR technology involves injecting carbonaceous biomass deep into the earth where it will be trapped in formations, it could be considered as an act of carbon sequestration. This has many benefits in cases if/when/where there would be a “carbon tax” on oilfield production as this technology would offer a carbon offset to reduce these costs. Even if there is no carbon tax in the jurisdiction of the production, the carbon sequestration could be sold as a carbon offset to another company, or different region of operation of the same company, which needs to purchase a carbon offset. This could also be used in public relations to market the company as a cutting-edge and environmentally responsible energy producer.

In an embodiment, this well treatment composition claims direct application of biomass in EOR or drilling fluid applications, without processing or treatment. In another embodiment, this well treatment composition claims denaturation of biomass before direct use or mixing without components. In one or more embodiments, denaturation comprises modifying the pH using acids or bases; surfactants additions; and temperature and/or pressure changes which may be abrupt.

In an embodiment, the solids can be at least partially removed or reduced before or after denaturation through filtration, sedimentation, centrifuge, or other process if needed if they are excessive or detrimental. The need for this step may depend on the specific biomass and application, but was not required to generate promising results in the experiments shown. The EOR agents may be used in tertiary recovery after water flooding as is typically done in polymer flooding. In other embodiments, the EOR agents be added to the water flooding recovery phase and may eliminate the need for a tertiary recovery. There could be applications for these agents as a viscosifiers, completion fluid additives, or substitutes for starch or xanthan gum.

In an embodiment, alkaline denaturation of microalgal biomasses is performed. These biomasses can be used in ambient conditions or under “downhole” conditions where higher temperatures, pressures, and shear rates may be encountered. More specifically, since denaturation is a thermally activated process, lower denaturant concentrations, lower mixing temperatures, and lower reaction times will likely be optimal for actual oilfield implementation.

### EXAMPLES

To provide a better understanding of the foregoing discussion, the following non-limiting examples are offered. Although the examples can be directed to specific embodiments, they are not to be viewed as limiting the invention in any specific respect.

Three different denaturants were tested for their ability to convert algal biomass into an effective EOR fluid. The denaturants used in these experiments are sodium hydroxide, citric acid, and urea. The sodium hydroxide and citric acid were included to test the effectiveness of denaturing under alkaline and acidic conditions, respectively. Urea was tested as a naturally-occurring chaotropic denaturant. None of these are expensive chemicals, and very little is used relative to the volume of EOR fluid generated.

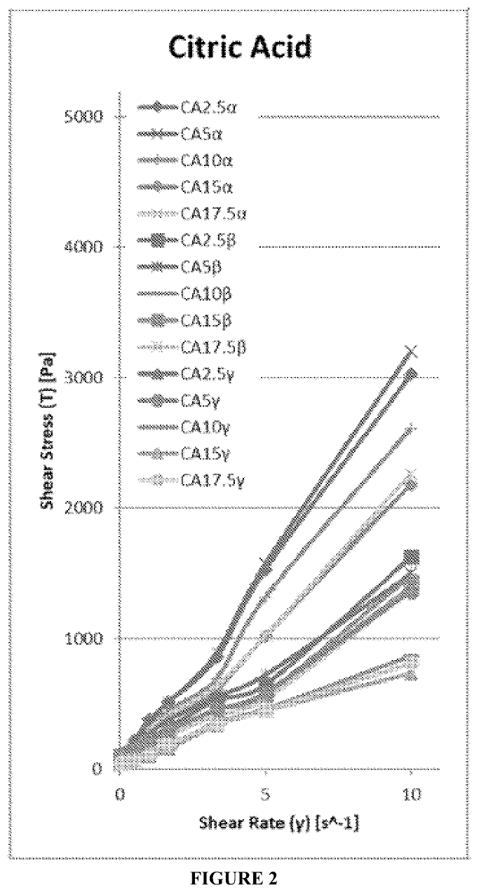

Spirulina algae meal (45-55% protein) was suspended in water and then chemically denatured using three different compounds in order to unravel proteins and create a slurry. Using different volumetric combinations of denaturant and water, 45 different EOR solutions were generated. Each sample of EOR fluid is made with 70 grams of Spirulina powder in various amounts of water as shown in FIG. 2. The denaturants are composed of 1 molar solutions that were added in quantities of 2.5, 5, 10, 15, and 17.5 mL per sample. The well treatment compositions are prepared by mixing on a magnetic stir plate. The dry algae powder is added slowly to the water while stirring until fully incorporated. Next, the denaturant is added to the solution, and the sample is allowed to mix for one hour. Afterward, the EOR fluid undergoes a series of tests.

The well treatment compositions were tested for displacement efficiency in sand packs. In order to approximate the environment of a reservoir, and since most sand is acquired naturally, e.g. from an unconsolidated formation (that is, unstratified sediment which has not yet become rock) which would later become sandstone, all-purpose sand will typically fit a normal distribution or grain size curve to better resemble a typical sandstone formation. This sand was poured into clear acrylic tubing capped with 200-mesh sieves set in PVC plugs which step down to brass bushings used for connecting to reciprocating pumps. Once packed so that the sand was immobilized, the columns were capped and weighed dry.

Reciprocating pumps were operated at an initial rate of 5 mL/min and increased slowly to 25 mL/min. The initial low rate is used to ensure there is no leakage, and also to minimize shifting of the sand as new material is introduced into the matrix. The maximum rate was capped at 25 mL/min in order to minimize channeling effects. At higher rates, the injected fluid may force separation of the sand matrix in order to accommodate the new volume, creating “channels” or cracks in the packed sand. By pumping at a sufficiently slow rate, the injected fluid achieves a cleaner sweep of the matrix and is able to contact nearly all sand particles. It is for this reason that clear acrylic tubing is employed: so that proper packing and proper flow without channeling can be visually confirmed with every sand pack. A channeling-free sweep is visually confirmed with every sand pack at every step. If the sand within the tube exhibits a major channel or fissure, it is discarded, and the test is performed again with a new sand pack.

Oil-bearing sandstone reservoirs are separated into three layers: gas-bearing, oil-bearing, and water-bearing. The top layer, known as the “gas cap,” puts pressure on the fluids and keeps vapors saturated in both the oil and water. However, when oil is produced, the formation is perforated just above the oil-water contact, and gravity, the weight of the formation above, and the pressure of the gas all push to produce oil. However, as oil is produced, the gas cap expands to fill the void. Once the gas in the formation reduces past the bubble point, the gas is no longer under sufficient pressure to retain vapors. Beyond this point, the gaseous pressure is no longer able to drive the oil out, and the oil, now without vapor, is considered “dead oil.” This is the point at which natural flow stops, and secondary production (water flooding) begins.

When oil is produced beyond the point of natural flow, water injection is employed to displace oil. However, the injection of liquids takes time to work its way through the pores of the formation, and the production pumps cannot realistically match rate of water absorbed into the formation. As a result, there is a discrepancy between the volume of water injected and the amount of fluid produced. This creates a phenomenon known as “water coning,” in which brine from below the oil/water contact is sucked up into the production tubing. Therefore, when tertiary production commences, there is brine mechanically mixed in with the oil.

In order to model the environment of oil-bearing sandstone reservoirs, packs were then saturated with a 25,000 ppm brine, which has a known density of 1.028 g/cm³ via the reciprocating pump. The samples are then weighed wet, and the difference in the weights is divided by the known density of the saline solution to determine the pore volume for the calculation of porosity.

Packs are then flooded with hydraulic oil, which has physical properties similar to “dead” crude oil (without vapors) but does not degrade to nearly the same extent as crude oil. This oil is pumped into one end of the sand pack and continues to be pumped until oil begins to exit the other end of the pack. A volumetric displacement is used to determine the oil saturation (generally in the 85% range) and brine saturation, also called irreducible water saturation. Because there are no vapors in the oil, both fluids are considered incompressible and a β value (oil compressibility factor) of 1 may be assumed, yielding a 1:1 displacement. At this point, the sand pack is structurally similar to a sandstone reservoir above the oil/water contact.

Keeping with industry standards, the pack is injected with pure water and production is continued until produced samples, being measured once per minute, reach a 50% water cut. This is to say that the volume of produced fluid within the time interval is 50% water or more. This is on the lower end of critical water cut, which may be as low as 50% or as high as 99%, depending on the operating conditions and price of crude on the open market. However, for the purposes of this research, 50% is taken to be the assumed critical water cut. After this water flood, the sand pack is injected with the EOR solution.

The EOR fluid is prepared by mixing on a magnetic stir plate. The water is spun up to a vortex, then the dry algae powder is added slowly until fully incorporated. Next, the denaturant is added to the solution and the sample is allowed to spin for one hour.

Finally, the EOR solution to be tested is injected into the sand pack in the amount of two pore volumes, beginning at 5 mL/min and slowly increased to 25 mL/min, after flooding the sand packs with a saline solution, dead oil (that is, devoid of suspended gases), and fresh water to a 50% water cut. The resulting slurry is allowed to separate out by gravity over a period of 2-3 days, generating three distinct layers: free oil at the top, oil/protein/water agglomerate in the middle, and a diluted polymer mixture in water at the bottom. The free oil volumes are measured for calculation purposes, though there is certainly more oil in the middle agglomerate.

Oil recovery was quantified in several ways. The “water flooding recovery” represents the volume of oil recovered during water flooding relative to the volume of the original oil in place (% OOIP). The “polymer recovery” represents the volume of oil recovered during EOR fluid flooding relative to the volume of oil originally in place (% OOIP). The total recovery is simply the sum of the water recovery and the polymer recovery. The inventors prefer to also report the percent of oil recovery that occurs during polymer flooding relative to the oil in the sand pack before polymer flooding (% RORP) which more directly shows the effect of the polymer by accounting for the amount of oil available to be produced in this stage. Using the % RORP minimizes the variance in the results arising from the variance in the water flooding recovery and serves to isolate the effect of the EOR fluid. The results are summarized in Tables 1a, b, c.

Oil recovery was calculated by post-flood recovery values that represent the volume of oil recovered during the EOR fluid injection divided by the volume of oil present immediately before EOR fluid injection (but after water flooding). This eliminates the variance in the recovery due to the differing amounts of oil remaining after the water flood and better isolates the effect of the EOR fluid.

The % Oil Recovered is defined as below.

$$\% Oil Recovered = \frac{Oil recovered by EOR fluid flood}{Oil present when EOR fluid is injected}$$

The calculated mixture measurements denote the pH of a simple dilution between the measured pH of the 1 molar denaturants and the water (tap). The theoretical mixtures denote the pH of perfect 1M denaturant solutions and the water (tap and deionized). The formulas used to calculate these pH trends are shown below:

$$pH of alkaline solution = 14 - {pOH}_{soln}$$

$${pOH}_{soln} = - \frac{Log ( {{\sum }_{i = 1}^{∞} [ {OH}^{-} ]}_{i} * {V}_{i} )}{{\sum }_{i = 1}^{∞} {V}_{i}} ;$$

$${[ {OH}^{-} ]}_{i} = {OH}^{-} concentration of component i$$

$${V}_{i} = partial volume of component i$$

$$pH of acidic solution = - \frac{Log ( {{\sum }_{j = 1}^{∞} [ {H}^{+} ]}_{j} * {V}_{j} )}{{\sum }_{j = 1}^{∞} {V}_{j}} ;$$

$${[ {H}^{+} ]}_{j} = {H}^{+} concentration of component j$$

$${V}_{j} = partical volume of component$$

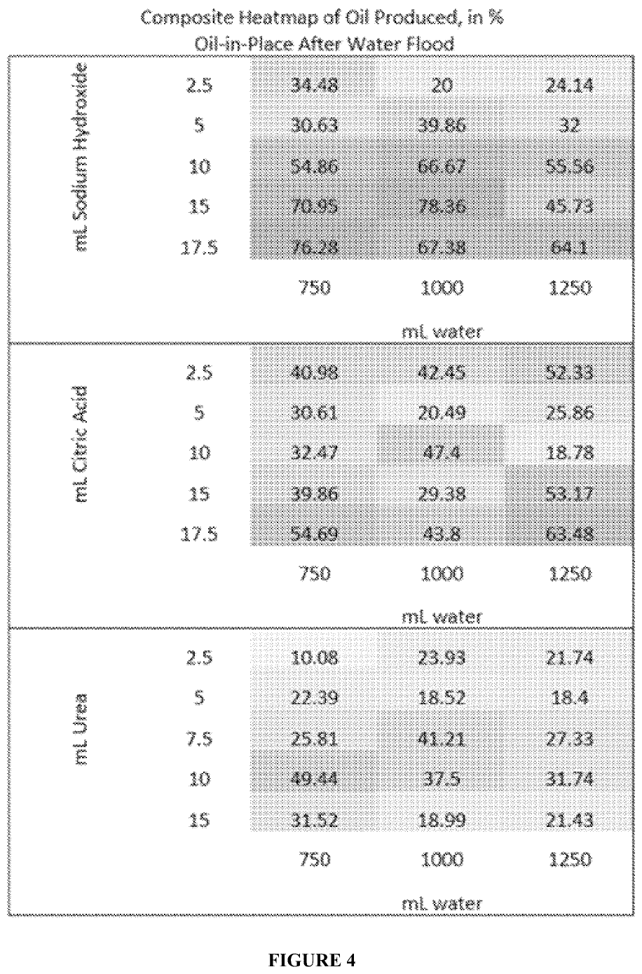

The results from the EOR experiments are presented in FIG. 4 and are shaded in order to provide a visual heat map. Values are shown as percentages, with darker colors indicating higher oil recovery values. While the chart for urea does not have a statistically significant correlation of increased oil recovery with respect to increased concentrations of denaturant (p=0.132), the charts for citric acid (p=0.021) and sodium hydroxide (p=0.048) do. The charts for sodium hydroxide and citric acid show higher production oriented toward more denaturant and less water. In the case of the urea samples, the peak oil recovery values occur with moderate amounts of denaturant added.

The composite pH chart (FIG. 31) shows interactions between the water, denaturants, and algae, respectively. If no chemical reactions occurred, the pH fluctuations would show nearly identical pH values and trends to that of theoretical dilutions of the materials. Deviations of these trends and values suggest that significant chemical reactions occur which can be attributed to the denaturation of the algae proteins, as well as other chemical reactions between denaturant and water impurities and the biomass.

To investigate the correlation between EOR and pH, the data observed in FIG. 31 was fitted with a linear trendline. It is evident that higher recoveries are highly correlated with the movement of pH from a neutral value when the denaturant is an acid or a base (p=0.037 for citric acid and p=0.002 for sodium hydroxide). The EOR mixtures containing urea do not yield very high recovery values and are accompanied by low changes in pH and did not show a significant correlation of oil recovery with pH (p=0.23). The EOR mixtures containing citric acid do yield relatively higher recovery values, but the dependency on citric acid concentration is non-linear with higher EOR values being achieved with low and high concentrations of citric acid. Lastly, the EOR mixtures containing sodium hydroxide have proved very effective and reliable in regards to yielding increasing EOR % values as the pH also increases. The trendline slope for the EOR mixture containing sodium hydroxide is notably larger than the trendline slope for the citric acid EOR mixture, which may be due to the alteration in rock wettability by the EOR fluid, the shear thickening effects exhibited in these well treatment compositions, the effectiveness of NaOH as a denaturant, or a combination of these mechanisms

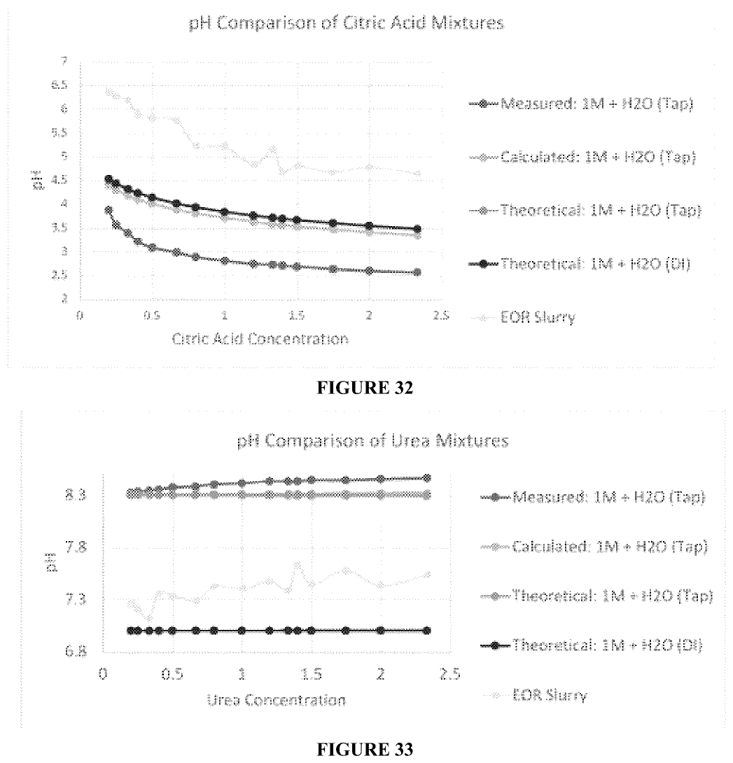

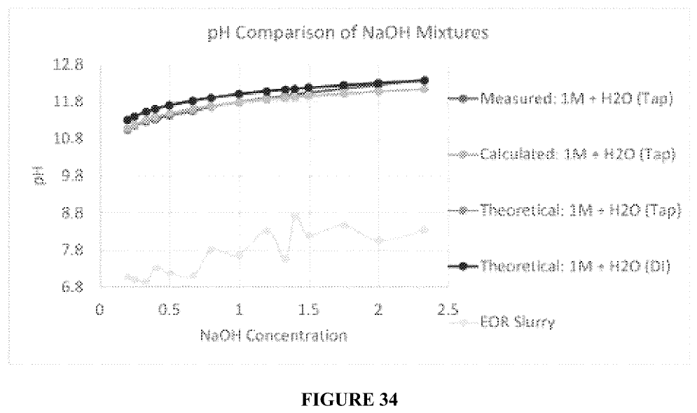

The pH comparison diagrams (FIGS. 32-34) show that there are significant chemical reactions between the denaturants and the algae. In mixtures of algae and water (absent of denaturant) the pH of the solutions α, β, and γ are: 6.67, 6.80, and 6.87, respectively. Thus, there is a slight reaction between the water and algal biomass, yielding a slightly acidic solution. If no significant chemical reactions were present, the pH values and trends of the EOR mixtures would remain almost identical to the theoretical dilution mixtures (absent of algae; see pH comparison charts), where no significant ion exchange takes place. As shown by the pH comparison charts, the values and trends of the EOR mixtures do not match their respective trends that are absent of algae, which depicts the presence of ion exchanges and ultimately denaturation of the algae within each EOR mixture.

Additionally, the diagrams denote the presence of chemical reactions occurring between the denaturants and tap water (in mixtures with an absence of algae), which can be seen by comparing the measured trend with the theoretical trends and calculated trends. Water and denaturant mixtures all show deviation between the measured pH values and the theoretical trends: Mixed only with tap water, sodium hydroxide mixtures showed a slight deviation in pH, urea mixtures showed a moderate deviation in pH, and citric acid mixtures showed large a deviation in pH. Additionally, the diagrams denote that the sodium hydroxide and citric acid show little to no signs of any significant interaction between the denaturants and tap water (in mixtures with an absence of algae), which can be seen by comparing the measured trends (denaturant and tap water) with the theoretical trends (denaturant and DI water), respectively.

FIGS. 32-34 denotes the presence of interaction between the tap water and the applied 1M urea solution, due to the relatively larger change in pH of the 1M urea+tap-water mixture than a urea+DI-water mixture. Water and denaturant mixtures all show deviation between the measured pH values and the theoretical trends: Mixed only with tap water, sodium hydroxide and citric acid mixtures showed a slight deviation in pH, while urea mixtures showed a moderate deviation in pH for the urea+tap-water mixtures. The deviation in urea may occur from interaction with any residual buffers within the tap water, interactions with dissolved inorganic components (such as calcium or magnesium), and changes in the amount of hydrogen bonding between urea molecules and water molecules, or interactions from residual isocyanate or ammonia within the 1M urea mixture.

The rheological properties were also characterized as shown in FIGS. 1-3. Shear-thickening (dilatant) fluids are known to have positive EOR effects, so this mechanism may be largely or partially responsible for the effectiveness of this EOR fluid.

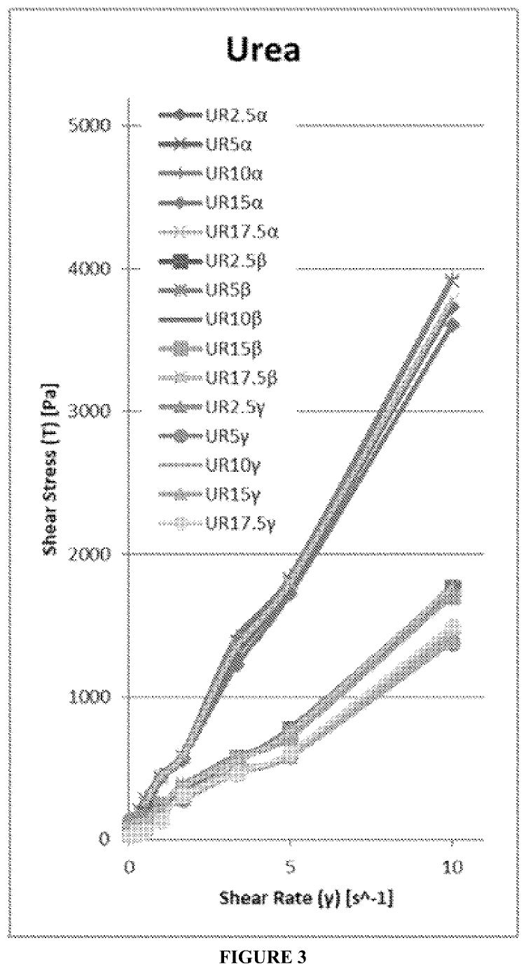

Each rheology chart shows how the amount of water present directly influences the relationship between shear rate and shear stress. All the polymer solutions tested seem to show dilatant trends in certain regions, though plotting the viscosity as a function of shear rate (FIG. 3) shows that the behavior is rather complex.

These rheology diagrams show that the EOR solutions showed dilatant tendencies over different ranges of shear rate. The increasing slopes of the shear stress with shear rate suggest that solutions in less water (α) are more dilatant than those in more water (γ), as shown by the clustering of α, β, and γ solutions yielding three distinct orientations within each fluid group. This is corroborated by the viscosity increases.

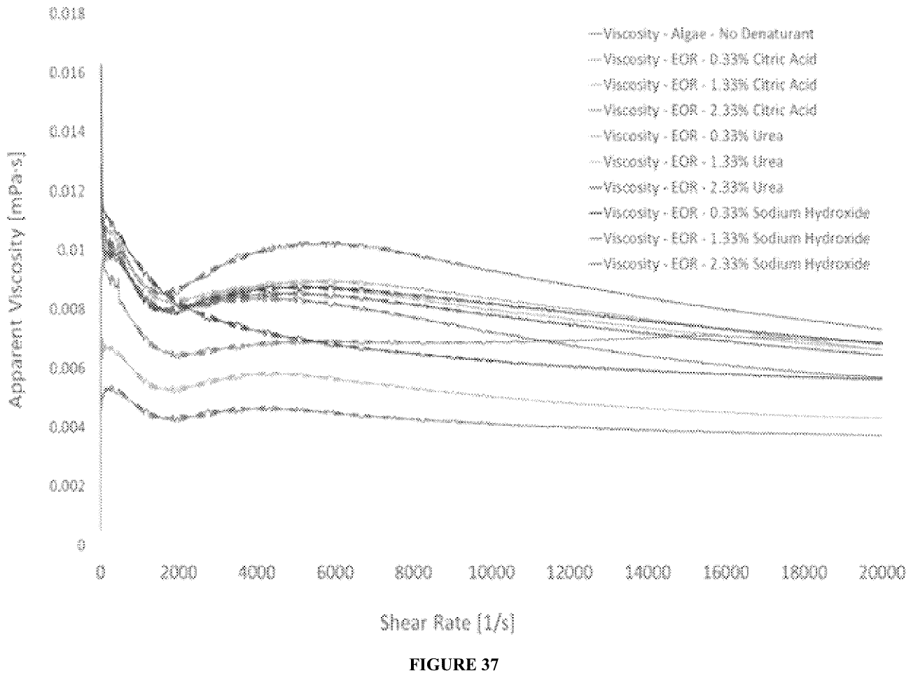

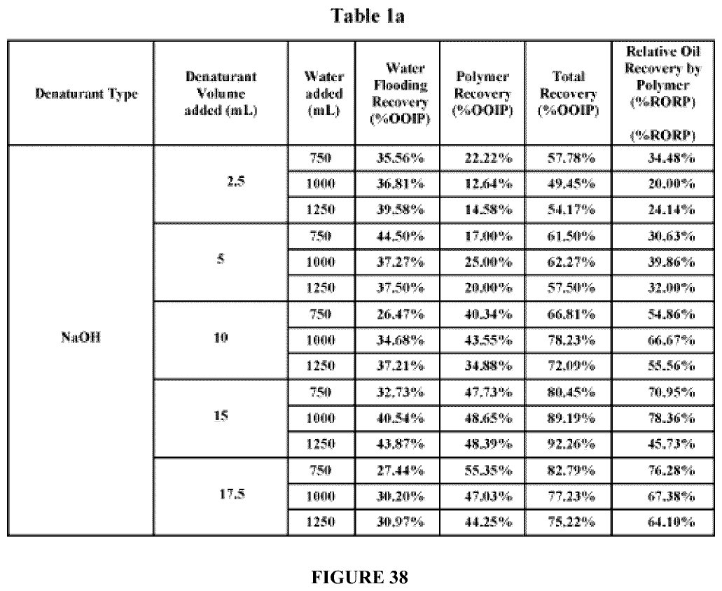

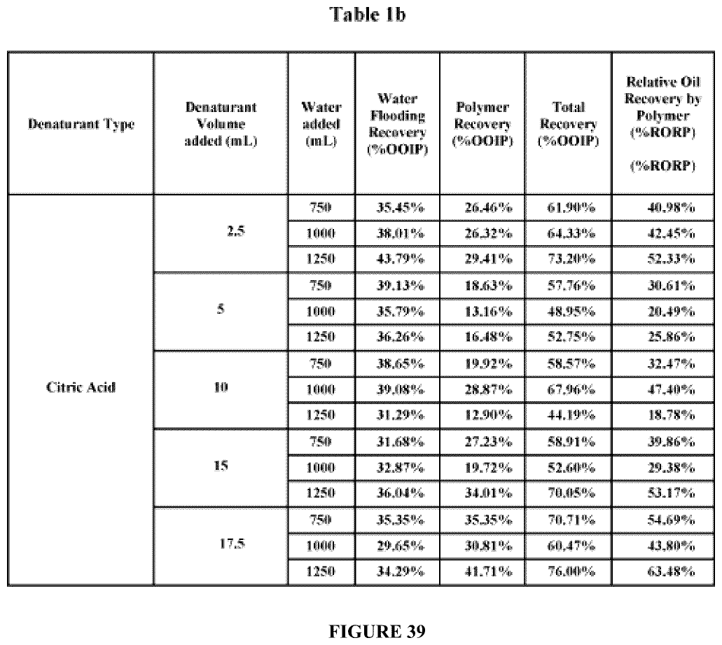

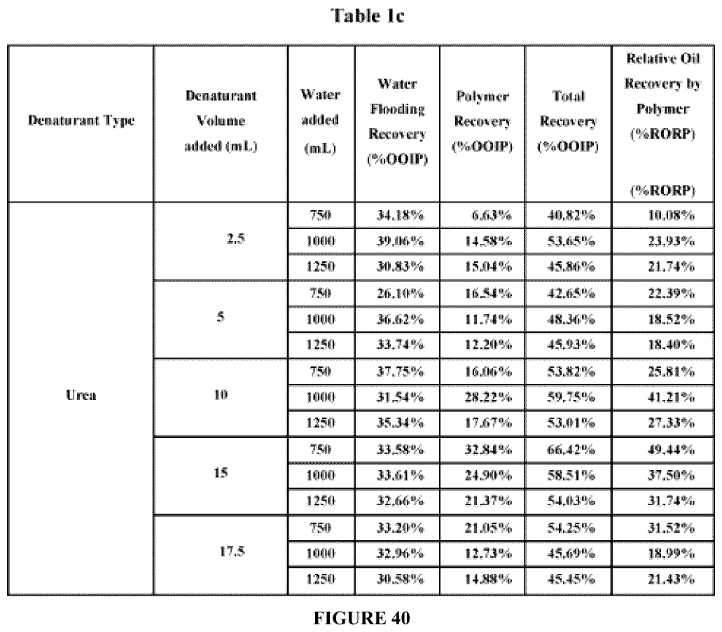

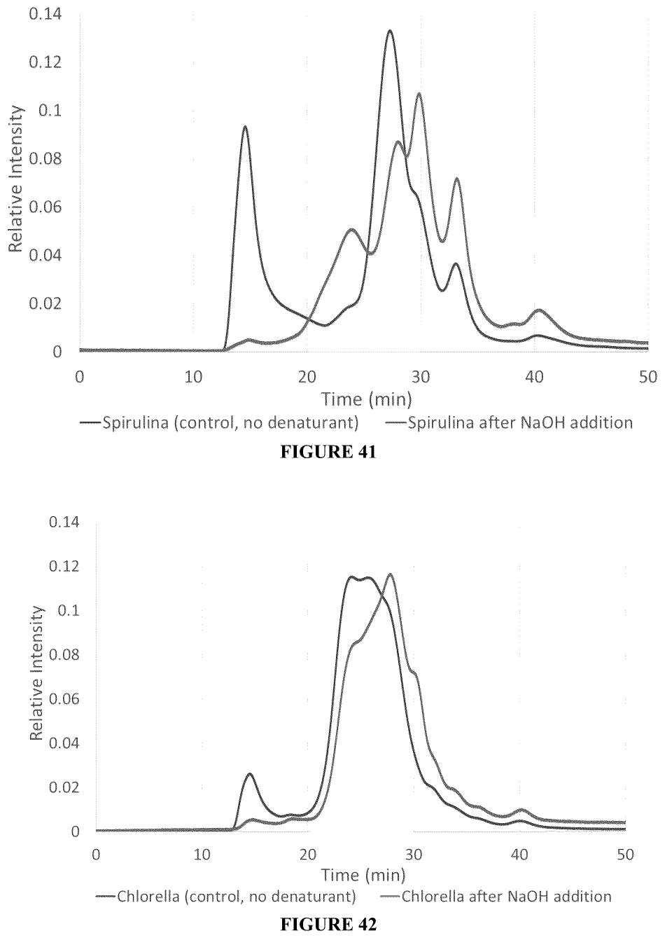

FIG. 37 shows the apparent viscosity increases with NaOH concentration and decreases with citric acid concentration. Many EOR agents work through a viscosifying effect, so the high apparent viscosities of the NaOH solutions are likely contributing to their effectiveness. These rotational results were collected on an Anton-Paar MCR 302 rheometer using a parallel plate geometry.

The ranges of shear-thickening change can be observed based on formulations. The EOR formulation can be modified to increase or decrease the flow through a given por size under specified conditions based on the location of the shear-thickening region.

As the composite heat map (FIG. 4) shows, the sodium hydroxide output is much higher than that of either citric acid or urea. This is likely because it was more effective in denaturing the algae. The discrepancy between the sodium hydroxide and citric acid results may also be explained via the phenomenon of alkaline flooding. This well-documented effect, while not well understood, states that alkaline fluids cause the surfaces of capillaries in reservoirs, if oil-wet, to become water-wet. This phenomenon is a result of the surface condition of the formation changing due to the properties of the substrate: the hydroxide ions from alkaline material increases the negative charges on silica and negates the positive charge on any clays in the formation. This results in a large sum of the oil that is untouched by water flooding to be released into the larger pores, increasing production. However, many of these capillaries are networked and could not be reached if the polymer favored the larger pores.

The rheology diagrams (FIGS. 1-3) show that the polymer solutions tested are dilatant. The diagrams also show that solutions with higher concentrations of protein are more dilatant. This, when observed in light of the first heat map, suggests that dilatant fluid properties aid in the production of oil. The reason dilatant fluids aid in oil production is their shear thickening effect. When a fluid is shear thickening, its resistance to motion increases with shear rate. This results in a fluid which becomes more viscous when moving at higher velocity in large pores, thereby directing more flow into smaller pores. This allows a more even sweep of fluid in the formation with fewer pores being bypassed by the shear thickening EOR fluid.

Important to note is that the oil values shown in these tables are “free” oil values. That is to say that the oil measured is the oil which has separated from the mix by gravity after a period of days. There is, however, more oil in mixture.

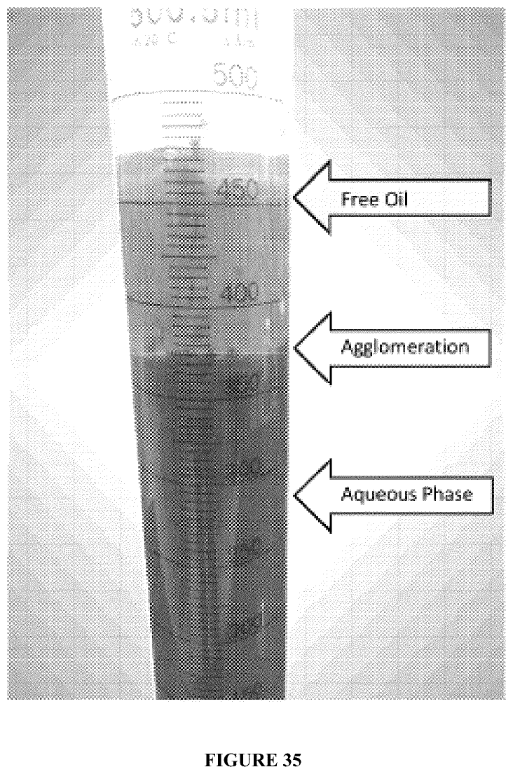

As displayed in FIG. 35, the free oil is separated from the dilute polymer mix below by an agglomeration of oil, water, and polymer. A close inspection shows that the interface is not flat; therefore, the mixture is not a pure liquid. The striations on the side suggest that it likely contains precipitated solids. The agglomeration is presumed to be oil suspended in precipitated protein, since it only develops when the EOR fluid is mixed with saltwater and oil, and not in the absence of salt.

It was also visually observed that oil is produced from the sand pack first, and once water begins to emerge, very little oil is produced afterward. This suggests that this heterogeneous mixture may be avoided if a new container is used to deposit the dilute polymer once the production line is expelling mainly water and polymer.

As expected, the water source significantly impacts the pH results. While deionized water is preferable for most laboratory experiments, tap water was used since deionized water would not likely be used in EOR formulations. The pH of the solutions was found to be significantly changed by the addition of algae, which indicates chemical reaction or interaction. The EOR effect seems to increase as the pH moves away from neutral. This result was expected since the proteinaceous biomass denatures as the pH moves away from neutral. A more detailed investigation into the specific chemical mechanisms of denaturation will need to be conducted in the future.

On the whole, comparing the aforementioned concentration groupings across the three fluid subsections of FIG. 4, well treatment compositions denatured with sodium hydroxide can be shown to exhibit a higher shear stress at any given shear rate than those denatured with urea, which in turn can be said to have a higher shear stress than those denatured with citric acid at any given shear rate. As such, well treatment compositions denatured with sodium hydroxide can be shown in FIG. 31 to have higher a higher viscosity than both those fluids denatured with urea and citric acid over the same intervals. Dilatant rheological behavior in EOR agents results in a fluid which moves more slowly in large pores, where it would normally flow at a higher rate, and moves relatively faster in smaller pores when compared to a Newtonian fluid. The added backpressure from the bulk of the fluid moving slowly in the large pores increases the flow into smaller pores, which increases the total surface area of the formation encountered by the EOR fluid.

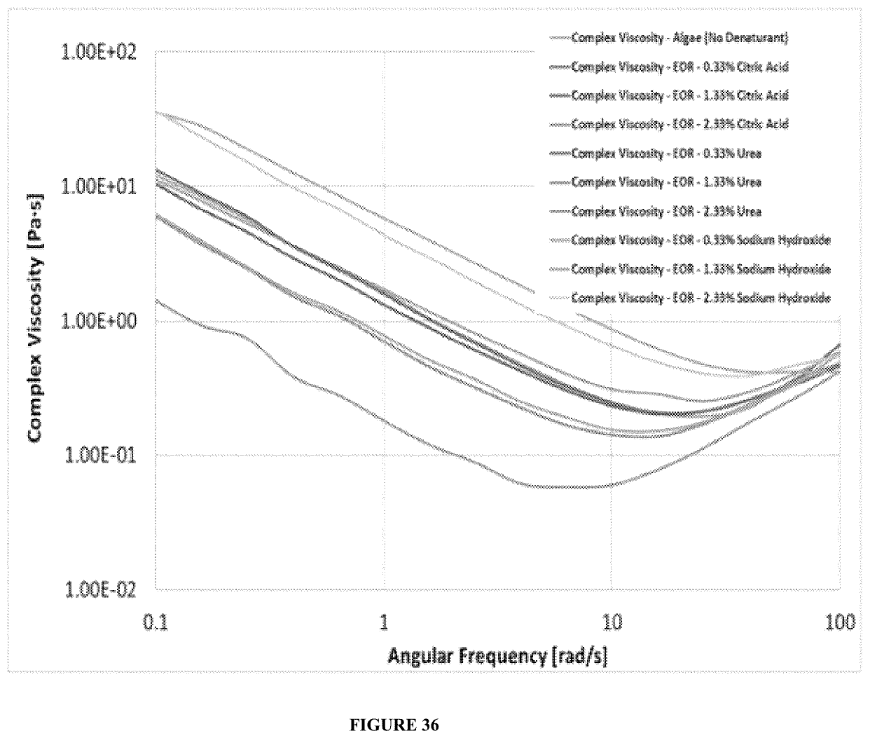

Oscillatory tests, such as this frequency sweep, also indicate the complex viscosity of the well treatment compositions exhibits a range of shear thickening that depends on the denaturant type and concentration. These results were collected on an Anton-Paar MCR 302 rheometer using a parallel plate geometry with an amplitude of 0.5%. A graph showing the results is at FIG. 36.

It is important to note that the rheology profile of these fluids is not shear-thickening over a broad range. The reality is more complex and interesting in that there is a shear-thinning behavior likely due to structural changes in the dissolved proteins at low shear rates, and a shear thickening effect likely due to the colloidal solids at higher shear rates.

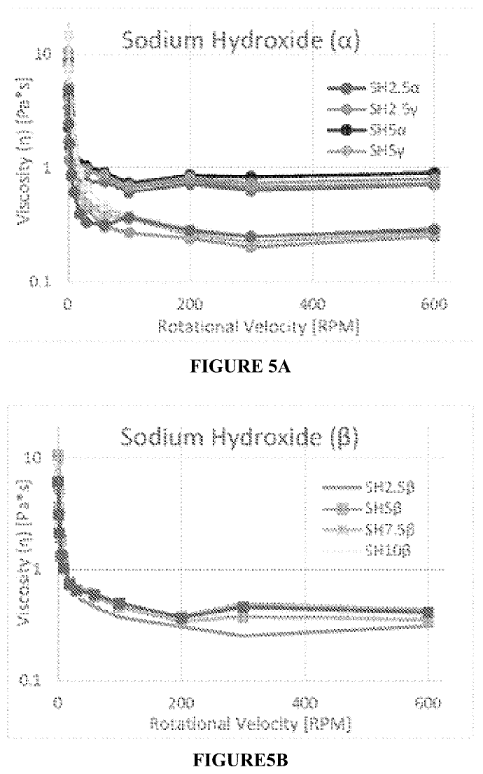

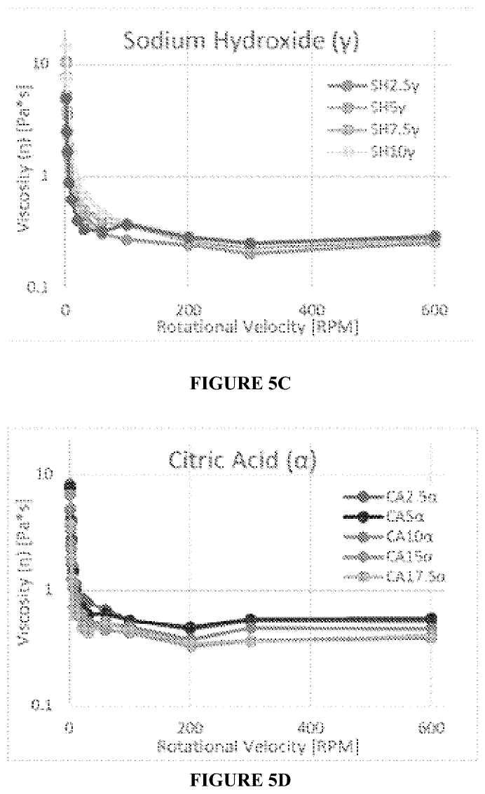

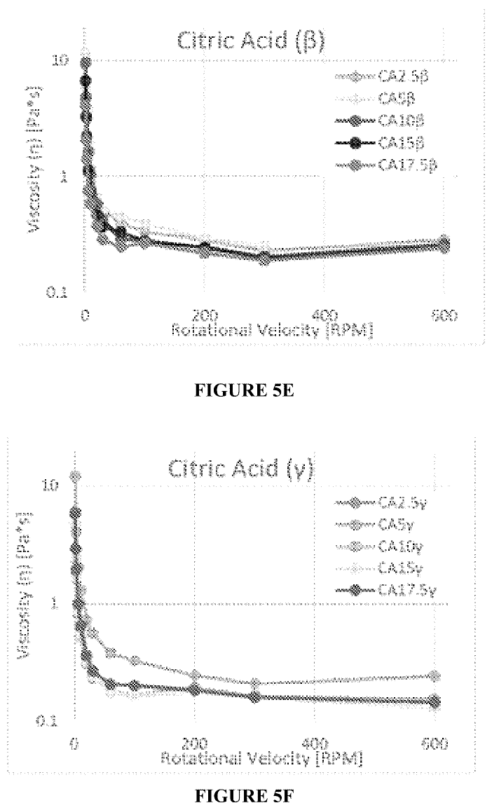

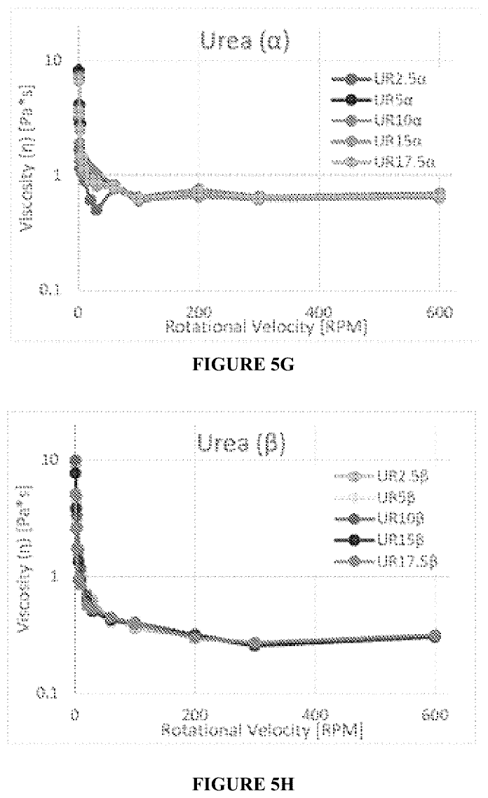

FIG. 5a-i displays the viscosity vs. shear rate measurements of all fluids tested, and the results do not fit any standard rheological model. As in FIG. 4, the results tend to split into three groupings which correspond with the concentration of algae with respect to water. There are regions of shear thickening displayed in the data, and these regions are reproduced in fluids with the same amount of added water. This shear-thickening region also moves reproducibly to different shear rates when different amounts of water are added, indicating that the region of shear thickening can likely be controlled and tailored to thicken at the desired shear rate for a particular application.

There are three plausible mechanisms that are likely to be the main drivers of the EOR effect of these fluids. First, all the fluids tested herein present a shear-thickening region in their rheological profile, which likely contributes to the overall increase in oil production of the system. Second, those fluids denatured with sodium hydroxide also benefit from the alkaline flooding effect which inverts the wettability of oil-wet capillaries and increases the overall yield. Third, all of the tested fluids have the benefit of a surfactant effect, as urea is itself a surfactant and alkaline flooding produces surfactants in situ both from naturally occurring “petroleum acids” and the decomposition of silica in the presence of hydroxides (Green & Willhite, 7.14 Alkaline Flooding, 2018). Citric acid produces surfactants by shifting the system to a lower pH.

Therefore, in the most successful EOR fluid tests, we propose the mechanisms of shear thickening, alkaline flooding, and surfactant activity contribute to the EOR efficacy. There are many drive mechanisms known to chemical flooding. However, the literature does not codify the types of mechanisms consistently, so one paper may claim a number of detailed drive mechanisms which may be encompassed by a single, broader mechanism claimed by another paper. Therefore, it is difficult to compare the mechanisms active in this system with others presented in literature, but we believe a novel combination of drive mechanisms cause the EOR effect in this fluid. With these mechanisms working in tandem, more interfacial surface area is contacted within the sand pack, more oil is expelled due to the sodium hydroxide samples' alkaline flooding properties, and the shear thickening behavior allows for oil to be pushed out with a more even sweep.

With a maximum efficiency of 78% of oil recovered from one injection of EOR solution (78% of oil in place after water flood was recovered by the EOR fluid injection. 96% of the oil originally in place was recovered with the water flooding and the EOR fluid injection combined), it is clear that well treatment compositions derived from algae-based biomasses have potential applications in the oilfield. The denaturing of waste proteins is an untapped reservoir of potential for generating production polymers with innate surfactant properties, negating the need for a surfactant sweep afterward, and has the potential for a very low cost when compared to other chemical flooding agents such as HPAM. Utilization of this waste material from biofuels and other bioprocessing operations in an EOR formulation could present a sustainably-sourced, environmentally-friendly, inexpensive, and yet effective solution for both conventional and emerging industries.

Of the three denaturants (sodium hydroxide, citric acid, and urea) tested, sodium hydroxide made the most effective well treatment compositions. The oil recovery reported was based on free oil volumes, and visual observation suggests that there is more oil produced in an agglomerated phase. This proteinaceous enhanced oil recovery is likely to operate by three mechanisms including shear thickening, alkaline flooding, and the surfactant effects.

Future research will test high-temperature, high-pressure systems, which will more closely model active reservoirs. Salinity effects will also be investigated as reservoirs can experience high variability in this area. There is also a need to determine the most effective method for breaking the emulsion produced below the free-oil line to increase oil recovery and recover clean water.

As shown in Table 2, the base drilling fluid components include a viscosifier, caustic soda (NaOH), defoamer, lignite, Desco, xanthan gum, and barite. All materials were measured by weight on laboratory balances and mixed in a stainless-steel mixing cup (FIG. 1a) using a five spindle, single speed Multimixer. Samples were aged in glass jars using a roller oven.

TABLE 2

| | | Concentration |
|---|---|---|
| Component | Function | (lb/bbl) |
| Water | Base fluid | Varied |
| Selected gel | Viscosifier | See |
| Organic polymer | Defoamer | 0.99 |
| Caustic soda | pH modifier | See |
| Lignite | Thinner | 23.1 |
| Desco | Deflocculant | 2.00 |
| Xanthan gum | Viscosifier | 0.50 |
| Barite | Weighting agent | Varied |

One lab barrel was mixed for each of the individual composition runs. A lab barrel is 350 milliliters and concentrations in pounds per barrel are roughly equivalent to grams per lab barrel as shown in Equation 1.

$$Lab barrel concentration conversion .$$

$$\frac{lbm}{bbl} = ( \frac{lbm}{bbl} ) ( \frac{453.592 g}{lbm} ) ( \frac{bbl}{158987.3 ml} ) ≅ \frac{g}{350 ml} \qquad Equation 1$$

All component concentrations were held constant except for the water, barite, gel, and caustic soda. Barite and water were only adjusted to ensure a final fluid density of ten pounds per gallon while the gel and caustic soda concentrations were varied according to Table 3.

TABLE 3

| Fluid ID | Gel NT (lb/bbl) | Algae (lb/bbl) | NaOH (lb/bbl) |
|---|---|---|---|
| L-1 | 20.0 | 0.0 | 0.25 |
| L-2 | 0.0 | 20.0 | 0.25 |
| L-3 | 0.0 | 30.0 | 0.25 |
| L-4 | 0.0 | 40.0 | 0.25 |
| M-1 | 20.0 | 0.0 | 0.50 |
| M-2 | 0.0 | 20.0 | 0.50 |
| M-3 | 0.0 | 30.0 | 0.50 |
| M-4 | 0.0 | 40.0 | 0.50 |
| H-1 | 20.0 | 0.0 | 1.00 |
| H-2 | 0.0 | 20.0 | 1.00 |
| H-3 | 0.0 | 30.0 | 1.00 |
| H-4 | 0.0 | 40.0 | 1.00 |
| Fluid ID | Gel NT (lb/bbl) | Algae (lb/bbl) | NaOH (lb/bbl) |

After measuring the proper amount of water into the mixing cup, the specified gel is slowly added while mixing. Then, the defoamer is added before allowing the gel to hydrate by mixing for thirty minutes. When the caustic soda is added, the fluid will thicken significantly, and the surface will no longer stir. At this point, the lignite is added to thin the fluid, followed by Desco. The xanthan gum and barite are added after ten and five minutes, respectively. After a final fifteen minutes of stirring, the mud is transferred to a glass jar and hot-rolled (aged) overnight in a roller oven for sixteen hours.

All rheology measurements were taken in accordance with API RP 13B-1 and manufacturer operating instructions using an OFITE Model 900 Viscometer.

Following hot roll, the fluid is mixed for five minutes before being added to the viscometer sample cup. The bob is submerged to the indicated line on the sleeve. The sample is heated to 120° F. and dial reading measurements are taken at 600, 300, 200, 100, 6, and 3 rpm speeds. Plastic viscosity and yield point are calculated using Equation 2 Rotation is stopped for a 10 seconds and the maximum initial dial reading at 3 rpm is recorded as the gel strength. This was repeated for 10 and 30 minutes.

PV=R₆₀₀−R₃₀₀ Equation 2: Calculation for plastic viscosity in centipoise.

YP=R₃₀₀−PV Equation 3: Calculation for yield point in lb/100 ft².

All fluid loss measurements were taken in accordance with API RP 13B-1 and manufacturer operating instruction using an OFITE 4-Unit HTHP Filter Press. After adjusting the filter press thermocouples to 250° F., the pressurized collection cell is filled to 0.5 inches below the top. A filter paper is placed on top of the cell before installing the cap and tightening all screws and valve stems. The cell is inserted into the preheated jacket apparatus with the filter paper side down. After adjusting the top and bottom regulators to 100 psi, the top valve stem is opened to apply back-pressure to the fluid while heating. Once the cell has reached temperature, the top regulator was adjusted to 600 psi and the bottom valve opened to begin filtration at a differential pressure of 500 psi. After 30 minutes the bottom valve stem can be closed, and filtrate is collected from the condenser. To correct to the standard API filter size, the filtrate volume is doubled and recorded as the total fluid loss.

All lubricity measurements were taken in accordance with manufacturer operating instructions using an OFITE EP and Lubricity Tester. Prior to testing a set of drilling fluids, a calibration check was performed on the OFITE lubricity meter by submerging the ring and block of the meter in deionized water while rotating at 60 rpm. Once the reading stabilizes, the torque on the meter was zeroed. Using the torque arm, 150 inch-pounds of force was applied for five minutes and the stabilized torque reading was recorded. The meter reading for deionized water was recorded and the correction factor (CF) was calculated using Equation 4.

$$Correction factor due to water reading .$$

$$CF = \frac{34}{Meter Reading for Water} \qquad Equation 4$$

After the calibration check, the sample container was filled with the first drilling fluid and the ring and block was submerged. The readings were allowed to stabilize and the torque was zeroed. 150 inch-pounds of torque was applied and the reading after 5 minutes was recorded. Equation 5 shows the coefficient of friction (CoF) calculation using the torque wrench reading of 150 inch-pounds and torque shaft lever arm length of 1.5 inches. This can be corrected using Equation 6 and the correction factor from Equation 4. For an easier comparison of different studies and lubricants, a better measure is the percentage reduction of torque shown in Equation 7.

$$Coefficient of friction calculation of OFITE lubricity meter .$$

$$CoF = \frac{Torque Reading}{( \frac{150 in - lb}{1.5 in} )} = \frac{τ}{100 lb} \qquad Equation 5$$

$$Correction of coefficient of friction with correction factor .$$

$${CoF}_{corr} . = ( CoF ) ( CF ) \qquad Equation 6$$

$$Calculation of reduction in torque in percent .$$

$${\%}_{red} = \frac{CoF - {CoF}_{base}}{{CoF}_{base}} ( 100 ) \qquad Equation 7$$

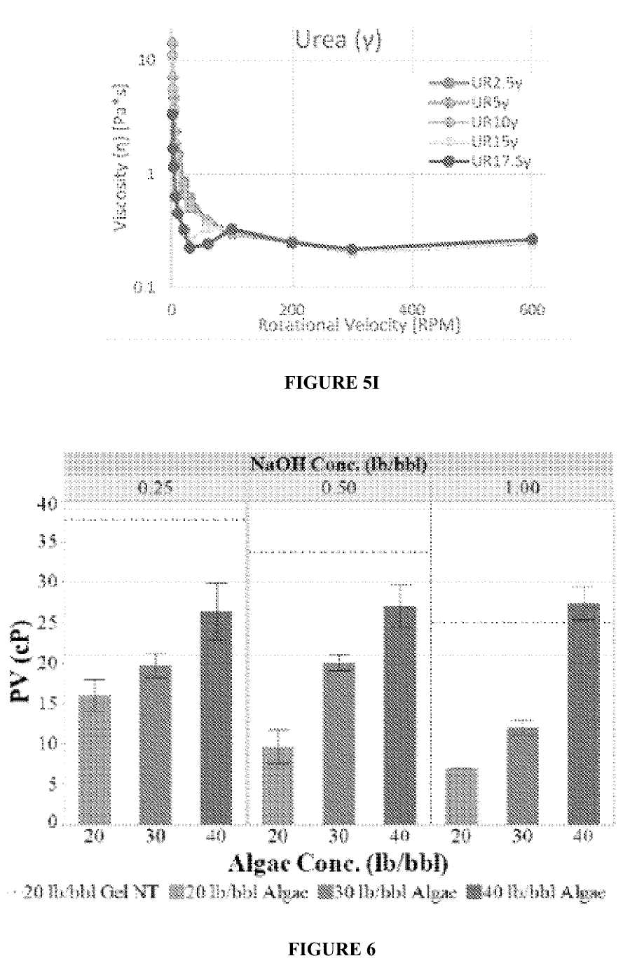

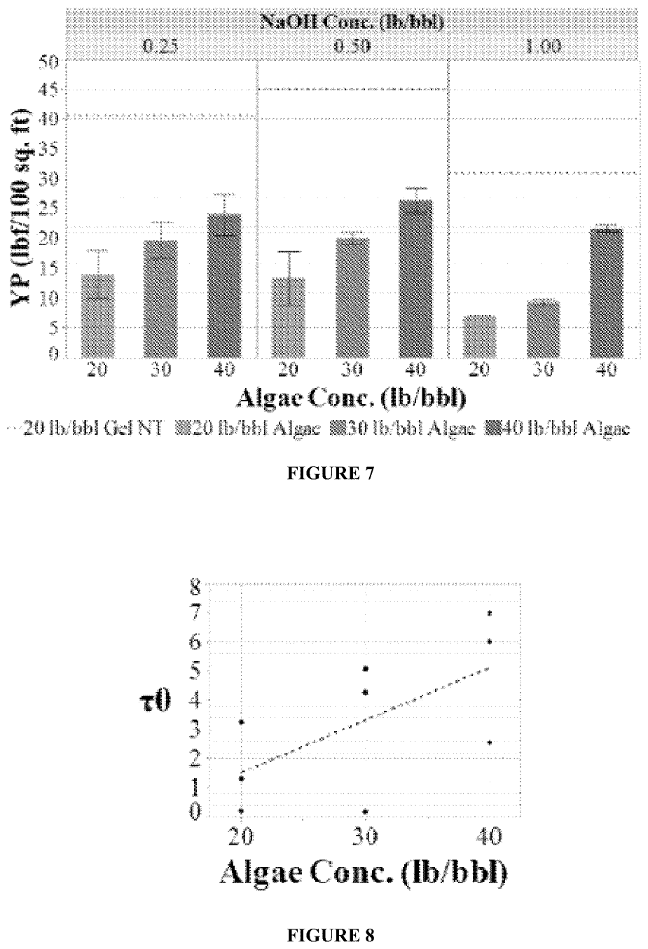

Bingham plastic is a common two-parameter model for non-Newtonian fluids and is the traditional method of fluid characterization in the field since the parameters can be quickly calculated without the use of a computer or complex algorithm. The plastic viscosity (PV) and yield point (YP) for each sample calculated using Equation 2 and Equation 3 are shown in FIG. 6 and FIG. 7. As seen in FIG. 6, the increase of algae concentration led to an increase in plastic viscosity across all caustic soda (NaOH) concentrations; however, the only sample that was able to match or surpass the twenty (20) pound per barrel bentonite (Gel NT) control was the forty (40) pound per barrel algae concentration at one (1) pound per barrel of caustic soda. As with plastic viscosity, yield point increased with increasing algae concentration. FIG. 7, 8 shows that none of the algae samples were able to achieve the yield point of their respective bentonite control. For plastic viscosity and yield point, the concentration of caustic soda did not have as significant of an effect on the values from low (0.25 pounds per barrel) to medium (0.50 pounds per barrel); however, there was a nearly fifty (50) percent drop in some cases when moving to the higher caustic concentration (1.00 pound per barrel).

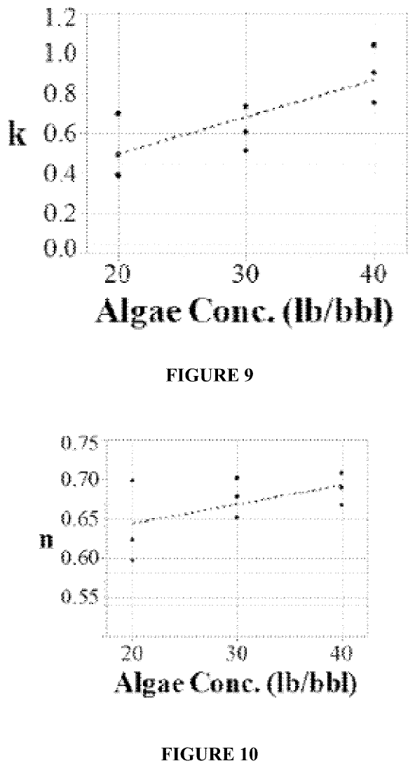

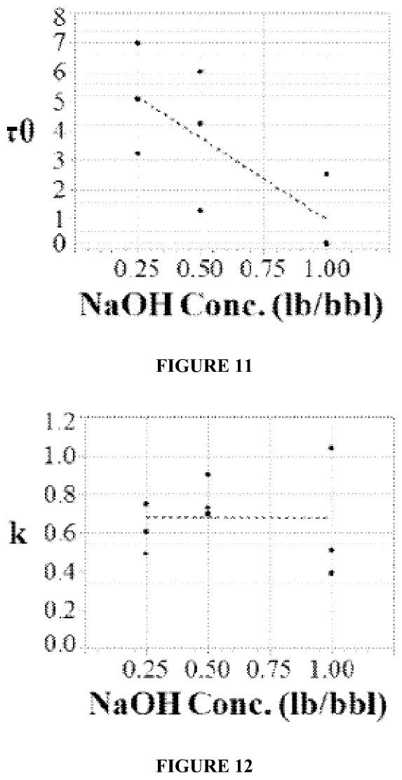

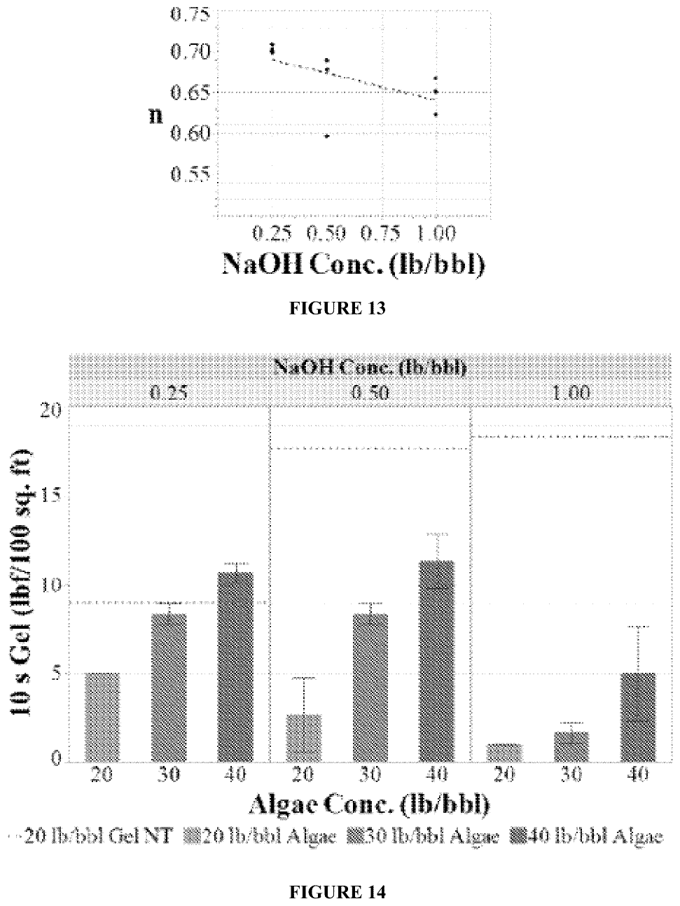

Though Bingham-plastic is commonly used for characterizing drilling fluid rheological properties, research has shown that Herschel-Bulkley, a three-parameter model for viscosity shown in Equation 8 is a better fit for the behavior of water-based bentonite drilling fluids. The rheology readings for all fluids with were fit to a curve using the Herschel-Bulkley model. The fit parameters are shown in Table 3 for each fluid and the fit of the parameters versus algae and caustic concentration are shown in FIG. 8, FIG. 9, FIG. 10, FIG. 11, FIG. 12, and FIG. 13. Based on this analysis, all three parameters have a positive correlation with algae concentration. Although k does not show a significant correlation with caustic concentration, τ0 and n have a negative correlation.

τ=τ₀+k\dot{γ}ⁿ Equation 8: Shear stress as a function of shear rate using the Herschel-Bulkley model.

TABLE 3.1
Herschel-Bulkley parameters fit for each fluid formulation

| Fluid ID | Gel NT (lb/bbl) | Algae (lb/bbl) | NaOH (lb/bbl) |
|---|---|---|---|
| L-1 | 20.0 | 0.0 | 0.25 |
| L-2 | 0.0 | 20.0 | 0.25 |
| L-3 | 0.0 | 30.0 | 0.25 |
| L-4 | 0.0 | 40.0 | 0.25 |
| M-1 | 20.0 | 0.0 | 0.50 |
| M-2 | 0.0 | 20.0 | 0.50 |
| M-3 | 0.0 | 30.0 | 0.50 |
| M-4 | 0.0 | 40.0 | 0.50 |
| H-1 | 20.0 | 0.0 | 1.00 |
| H-2 | 0.0 | 20.0 | 1.00 |
| H-3 | 0.0 | 30.0 | 1.00 |
| H-4 | 0.0 | 40.0 | 1.00 |
| Fluid ID | Gel NT (lb/bbl) | Algae (lb/bbl) | NaOH (lb/bbl) |

Table 3.2 shows the Mean Surface Tension Values of EOR Solutions and Baseline Fluids with 95% Confidence Intervals. The data was collected using the platinum DuNouy ring method with a KSV 702 Force Tensiometer. All of the EOR solutions significantly reduce the surface tension of the water, which may allow for greater penetration into porous media for improved oil recovery.

TABLE 3.2

| | Mean Surface | |
|---|---|---|
| | Tension | 2*Standard |
| Fluid | $( \frac{m N}{m} )$ | Deviation (95% confidence) |
| EOR Solution + No Denaturant | 46.60 | ±0.40 |
| EOR Solution + 0.33% 1M Citric Acid | 46.68 | ±0.24 |
| EOR Solution + 1.33% 1M Citric Acid | 48.15 | ±0.38 |
| EOR Solution + 2.33% 1M Citric Acid | 49.38 | ±0.34 |
| EOR Solution + 0.33% 1M Urea | 47.17 | ±0.62 |
| EOR Solution + 1.33% 1M Urea | 48.04 | ±0.22 |
| EOR Solution + 2.33% 1M Urea | 48.43 | ±0.38 |
| EOR Solution + 0.33% 1M Sodium | 46.52 | ±0.14 |
| Hydroxide | | |
| EOR Solution + 1.33% 1M Sodium | 47.33 | ±0.20 |
| Hydroxide | | |
| EOR Solution + 2.33% 1M Sodium | 47.25 | ±0.34 |
| Hydroxide | | |
| Tap Water | 75.81 | ±0.40 |
| Tap Water + 2.33% 1M Citric Acid | 68.86 | ±0.13 |
| Tap Water + 2.33% 1M Urea | 62.01 | ±0.08 |
| Tap Water + 2.33% 1M Sodium | 72.12 | ±0.63 |
| Hydroxide | | |
| Brine (25,000 ppm NaCl) | 76.01 | ±0.74 |
| Hydraulic Oil | 34.6 | ±0.06 |

Table 3.3 shows Mean Contact Angle Values of EOR Solutions and Baseline Fluids with 95% Confidence Intervals. The contact angles were determined via the sessile drop method using a Kruss advance drop shape analyzer—-DSA100. The contact angle between the EOR fluid and a quartz surface indicate how much the fluid spreads on the surface. Note the lowest EOR contact angle (2.33% NaOH) had the highest oil recovery. These values along with surface tension can be used to calculate work of adhesion.

TABLE 3.3

| | Mean Contact Angle | 2*Standard Deviation |
|---|---|---|
| Fluid | (° → radians) | (95% confidence) |
| EOR Solution + No Denaturant | 52.01 → 0.9073 | ±2.68 → ±0.0468 |
| EOR Solution + 0.33% 1M Citric Acid | 49.06 → 0.8558 | ±1.44 → ±0.0502 |
| EOR Solution + 1.33% 1M Citric Acid | 42.39 → 0.7395 | ±2.84 → ±0.0495 |
| EOR Solution + 2.33% 1M Citric Acid | 40.51 → 0.7067 | ±3.58 → ±0.0625 |
| EOR Solution + 0.33% 1M Urea | 46.69 → 0.8145 | ±3.00 → ±0.0523 |
| EOR Solution + 1.33% 1M Urea | 43.89 → 0.7656 | ±4.00 → ±0.0700 |
| EOR Solution + 2.33% 1M Urea | 40.55 → 0.7074 | ±2.72 → ±0.0474 |
| EOR Solution + 0.33% 1M Sodium | 47.75 → 0.8330 | ±3.76 → ±0.0656 |
| Hydroxide | | |
| EOR Solution + 1.33% 1M Sodium | 44.73 → 0.7803 | ±2.78 → ±0.0485 |
| Hydroxide | | |
| EOR Solution + 2.33% 1M Sodium | 40.35 → 0.7039 | ±2.98 → ±0.0520 |
| Hydroxide | | |
| Tap Water | 41.34 → 0.7212 | ±3.42 → ±0.0597 |
| Tap Water + 2.33% 1M Citric Acid | 39.62 → 0.6911 | ±2.96 → ±0.0516 |
| Tan Water + 2.33% 1M Urea | 40.51 → 0.7067 | ±3.34 → ±0.0583 |
| Tap Water + 2.33% 1M Sodium | 32.78 → 0.5718 | ±2.02 → ±0.0352 |
| Hydroxide | | |
| Brine (25,000 ppm NaCl) | 50.13 → 0.8745 | ±2.32 → ±0.0405 |
| Hydraulic Oil | 23.42 → 0.4085 | ±4.74 → ±0.0827 |

Table 3.4 shows Interfacial Tension Values of EOR Solution Components and Baseline Fluids Against Hydraulic Oil. The data was collected using the platinum DuNouy ring method with a KSV 702 Force Tensiometer. The values of the interfacial tension between EOR solutions and the recovery do not have a simple correlation. Citric acid may produce inferior (compared to NaOH) EOR agents due to the resultant interfacial tension. This high IFT may make it difficult for citric acid to penetrate oil-filled pores and to convey the oil.

TABLE 3.4

| Fluid | $Interfacial Tension ( \frac{m N}{m} )$ |
|---|---|
| EOR Solution + No Denaturant (suspension) | 2.26 |
| EOR Solution + No Denaturant (particulates) | 1.80 |
| EOR Solution + 0.33% 1M Citric Acid | 1.68 |
| (suspension) | |
| EOR Solution + 1.33% 1M Citric Acid | 1.71 |
| (suspension) | |
| EOR Solution + 2.33% 1M Citric Acid | 2.07 |
| (suspension) | |
| EOR Solution + 0.33% 1M Citric Acid | 12.81 |
| (particulates) | |
| EOR Solution + 1.33% 1M Citric Acid | 21.27 |
| (particulates) | |
| EOR Solution + 2.33% 1M Citric Acid | 60.27 |
| (particulates) | |
| EOR Solution + 0.33% 1M Urea (suspension) | 1.69 |
| EOR Solution + 1.33% 1M Urea (suspension) | 1.70 |
| EOR Solution + 2.33% 1M Urea (suspension) | 1.70 |
| EOR Solution + 0.33% 1M Urea (particulates) | 5.74 |
| EOR Solution + 1.33% 1M Urea (particulates) | 7.88 |
| EOR Solution + 2.33% 1M Urea (particulates) | 9.50 |
| EOR Solution + 0.33% 1M Sodium Hydroxide | 1.68 |
| (suspension) | |
| EOR Solution + 1.33% 1M Sodium Hydroxide | 1.67 |
| (suspension) | |
| EOR Solution + 2.33% 1M Sodium Hydroxide | 1.66 |
| (suspension) | |
| EOR Solution + 0.33% 1M Sodium Hydroxide | 1.82 |
| (particulates) | |
| EOR Solution + 1.33% 1M Sodium Hydroxide | 1.71 |
| (particulates) | |
| EOR Solution + 2.33% 1M Sodium Hydroxide | 1.70 |
| (particulates) | |
| Brine (25,000 ppm NaC1) | 4.03 |
| Tap Water | 5.95 |
| Tap Water + 2.33% 1M Citric Acid | 14.60 |
| Tap Water + 2.33% 1M Urea | 5.66 |
| Tap Water + 2.33% 1M Sodium Hydroxide | 2.37 |

Table 3.5 shows Volumetric Analysis of 50 mL Samples of EOR Solutions after Centrifuge. All of the EOR solutions were centrifuged to separated out the solids. The best performing EOR agent also had the lowest % solids. If there are significant amount of large particles, they can block pores and limit EOR. However, a dispersion of smaller particulates may yield a shear-thickening rheological effect to improve EOR.

TABLE 3.5

| | Suspension | Particulates | Volume Settled |
|---|---|---|---|
| Solution | Volume (mL) | Volume (mL) | (%) |
| EOR Solution + No Denaturant | 36 | 14 | 0.28 |
| EOR Solution + 0.33% 1M Citric Acid | 35 | 15 | 0.30 |
| EOR Solution + 1.33% 1M Citric Acid | 32 | 18 | 0.36 |
| EOR Solution + 2.33% 1M Citric Acid | 30 | 20 | 0.40 |
| EOR Solution + 0.33% 1M Urea | 36.5 | 13.5 | 0.27 |
| EOR Solution + 1.33% 1M Urea | 35 | 15 | 0.30 |
| EOR Solution + 2.33% 1M Urea | 33.5 | 16.5 | 0.33 |
| EOR Solution + 0.33% 1M Sodium | 35 | 15 | 0.30 |
| Hydroxide | | | |
| EOR Solution + 1.33% 1M Sodium | 36 | 14 | 0.28 |
| Hydroxide | | | |
| EOR Solution + 2.33% 1M Sodium | 44.5 | 55 | 0.11 |
| Hydroxide | | | |

Table 3.6 Mean Work of Adhesion Values of EOR Solutions and Baseline Fluids with 95% Confidence Intervals. The work of adhesion indicates the energy with which the EOR solution interacts with a quartz surface. A higher work of adhesion would indicate a higher energy available for the EOR fluid to displace oil from the sand's surface. While this does not correlate with the effectiveness of different denaturants, it does correlate with the increasing EOR effect with concentration for a given denaturant.

TABLE 3.6

| | Mean Work | |
|---|---|---|
| | of Adhesion | 2*Standard |
| Fluid | $( \frac{m N}{m} )$ | Deviation (95% confidence) |
| EOR Solution + No Denaturant | 74.98 | ±1.83 |
| EOR Solution + 0.33% 1M Citric Acid | 77.28 | ±1.81 |
| EOR Solution + 1.33% 1M Citric Acid | 83.72 | ±1.74 |
| EOR Solution + 2.33% 1M Citric Acid | 86.93 | ±2.09 |
| EOR Solution + 0.33% 1M Urea | 79.54 | ±2.08 |
| EOR Solution + 1.33% 1M Urea | 82.67 | ±2.35 |
| EOR Solution + 2.33% 1M Urea | 85.24 | ±1.64 |
| EOR Solution + 0.33% 1M Sodium | 77.81 | ±2.27 |
| Hydroxide | | |
| EOR Solution + 1.33% 1M Sodium | 80.97 | ±1.65 |
| Hydroxide | | |
| EOR Solution + 2.33% 1M Sodium | 83.27 | ±1.70 |
| Hydroxide | | |
| Tap Water | 132.75 | ±3.07 |
| Tap Water + 2.33% 1M Citric Acid | 121.92 | ±2.31 |
| Tap Water + 2.33% 1M Urea | 109.17 | ±2.36 |
| Tap Water + 2.33% 1M Sodium | 132.77 | ±2.70 |
| Hydroxide | | |
| Brine (25,000 ppm NaC1) | 124.76 | ±2.65 |
| Hydraulic Oil | 66.35 | ±1.14 |

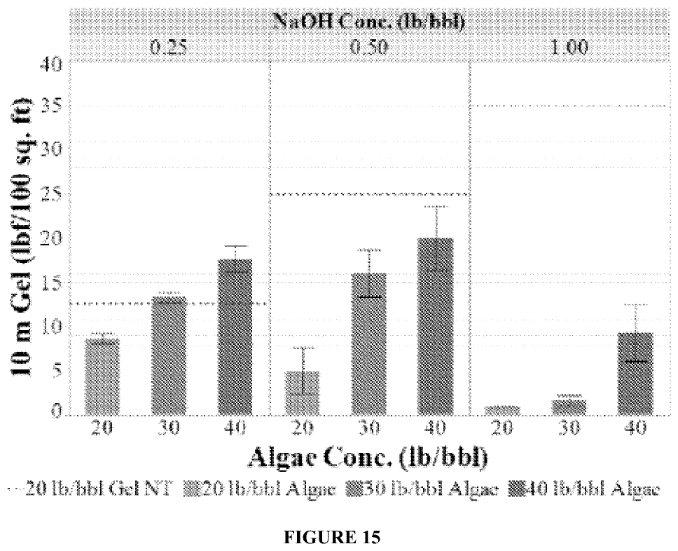

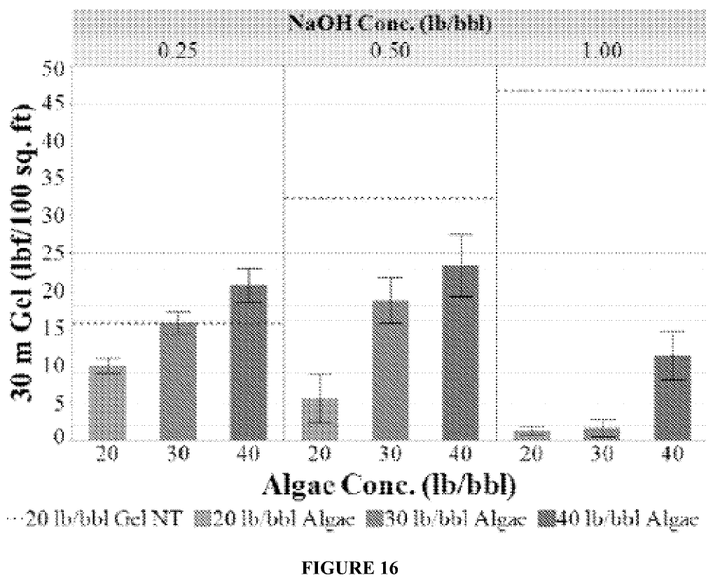

The gel strength is a measure of the ability of a fluid to maintain viscosity after a period of static rest. FIG. 14, FIG. 15, and FIG. 16 show the results of ten-second, ten-minute, and thirty-minute gel strength readings, respectively. All values are an average of triplicate tests and the error bars are one (1) standard deviation from the mean. By comparing the dotted control lines on all three gel strength graphs, it is evident that the gel strength performance for the bentonite control is significantly impacted by caustic concentration. Due in part to this, the thirty (30) pound per barrel algae sample performed similarly to the control and the forty (40) pound per barrel sample outperformed the control. Otherwise, the control outperformed the algae.

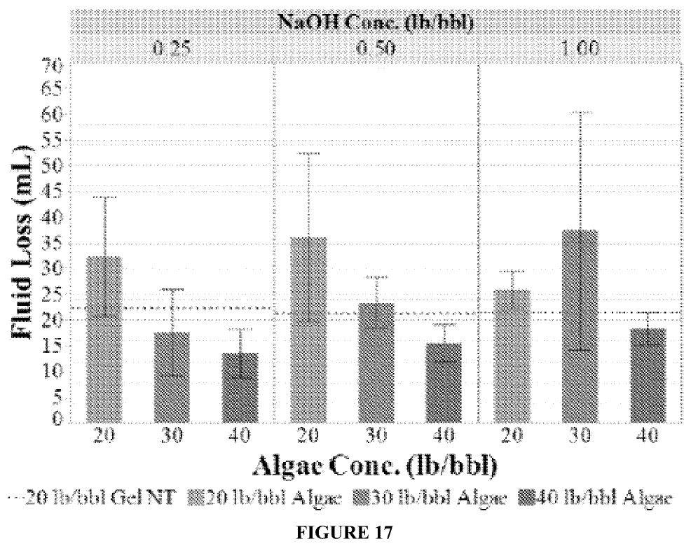

High pressure, high temperature filtrate volume is a laboratory measure of how well the sample can mitigate or reduce fluid losses into the pores of the formation. FIG. 17 shows the filtrate volume reported as fluid loss as described in the API procedure. All values are an average of triplicate tests and the error bars are one (1) standard deviation from the mean. The algae powder provided the greatest fluid loss reduction at a concentration of forty (40) pounds per barrel. Although this concentration outperformed the bentonite control at all three caustic concentrations, the best performance was seen at the lowest level (0.25 pounds per barrel).

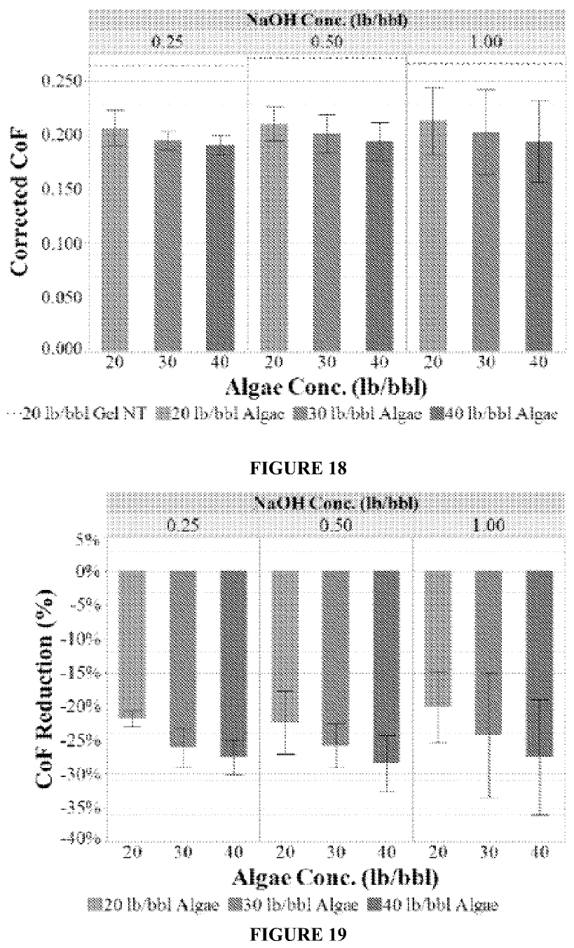

FIG. 18 shows the coefficient of friction for each sample corrected for the water reading as in Equation 6 using the correction factor from Equation 4. A more effective way to measure the performance of a lubricant is the reduction in coefficient of friction or torque from some control. The values for this reduction are shown in FIG. 19 calculated using Equation 7. Both figures show that an increase in caustic soda concentration correlates with an increase in coefficient of friction while an algae concentration increase leads to reduced coefficient of friction values. The worst algae sample performance still reduced the coefficient of friction by an average of twenty (20) percent compared to the bentonite control formulation; however, the reduction seen at forty (40) pounds per barrel of algae powder was the best at all three caustic concentrations with a reduction of over twenty seven (27) percent.

From these results, it appears that the denatured biomass does not have the proper rheological properties to replace bentonite in drilling fluids. However, it may help the rheological properties as an additive. While the denatured biomass did not outperform the control in regards to the rheological properties, it was found that the biomass was very effective at reducing fluid loss and increasing the lubricity. Hence, the denatured biomass holds great promise as an additive based on these latter two properties, which still being beneficial to the rheology.

The following lubricants are mentioned in this study for comparison: Lube A is a high-performance, non-EPA-approved lubricant (HDL Plus). Lube B is an EPA approved lubricant (CoastaLube).

As shown in Table 4, the base drilling fluid components include a non-treated bentonite gel (Gel NT), caustic soda (NaOH), defoamer, lignite, Desco, xanthan gum, and barite. The base fluid materials were measured by weight on laboratory balances and mixed in two (2) gallon batches using a Silverson L5M-A laboratory mixer like the one shown in FIG. 0.20. The lubricants were mixed in a lab barrel (350 ml) of base fluid in a stainless-steel mixing cup using a five spindle, single speed Multimixer as shown in FIG. 1a and FIG. 2a. Samples were aged in glass jars using a roller oven.

TABLE 4
Base drilling fluid component functions and concentrations.

| | | Concentration |
|---|---|---|
| Component | Function | (lb/bbl) |
| Water | Base fluid | Varied |
| Gel NT | Viscosifier | 20.0 |
| Organic polymer | Defoamer | 0.99 |
| Caustic soda | pH modifier | 0.5 |
| Lignite | Thinner | 2.00 |
| Desco | Deflocculant | 2.00 |
| Xanthan gum | Viscosifier | 0.50 |
| Barite | Weighting agent | Varied |
| Lubricant | Lubricant | See Table 0.5 |

Twenty lab barrels—approximately two gallons—of base fluid was mixed for each batch of testing. A lab barrel is 350 milliliters and concentrations in pounds per barrel are roughly equal to grams per lab barrel as shown in Equation 0.9. The additive concentrations were held constant except for the water, barite, and lubricant. Barite and water were only adjusted to ensure a final fluid density often pounds per gallon while the lubricant type and concentration were varied according to Table 5. After measuring the proper amount of water into the mixing vessel, the Gel NT is slowly added while mixing. Then, the defoamer is added before allowing the gel to hydrate by mixing for thirty minutes. When the caustic soda is added, the fluid will thicken significantly, and the surface will no longer stir. At this point, the lignite is added to thin the fluid, followed by Desco. The xanthan gum and barite are added after ten and five minutes, respectively. After fifteen more minutes of stirring, the mud is divided into lab barrels in stainless steel malt cups. The volume of fluid that will be replaced by lubricant is extracted by syringe. The selected lubricant is then added at the given volume (the algae weight was calculated using a measured specific gravity of 1.816). The mud is then transferred to a glass jar and hot-rolled (aged) overnight in a roller oven for sixteen hours at 150° F. (65.5° C.).

$$Lab barrel concentration conversion .$$

$$\frac{lbm}{bbl} = ( \frac{lbm}{bbl} ) ( \frac{453.592 g}{lbm} ) ( \frac{bbl}{158987.3 ml} ) ≅ \frac{g}{350 ml} \qquad Equation 0.9$$

TABLE 5
Additive concentration variations for
each drilling fluid formulation.

| Fluid | Lubricant | Concentration |
|---|---|---|
| ID | Type | (vol %) |
| 1 | None | 0.0 |
| 2 | Algae | 1.0 |
| 3 | Algae | 2.0 |
| 4 | Algae | 3.0 |
| 5 | Diesel | 1.0 |
| 6 | Diesel | 2.0 |
| 7 | Diesel | 3.0 |
| 8 | Lube A | 1.0 |
| 9 | Lube A | 2.0 |
| 10 | Lube A | 3.0 |
| 14 | Lube B | 1.0 |
| 15 | Lube B | 2.0 |
| 16 | Lube B | 3.0 |
| 17 | Algae & Lube B | 0.5 & 0.5 |
| 18 | Algae & Lube B | 1.0 & 1.0 |
| 19 | Algae & Lube B | 1.5 & 1.5 |

All rheology measurements were taken in accordance with API RP 13B-1 and manufacturer instructions using an OFITE Model 900 Viscometer.

Following hot roll, the fluid is mixed for five minutes before being added to the viscometer sample cup. The bob is submerged to the indicated line on the sleeve. The sample is heated to 120° F. and dial reading measurements are taken at 600, 300, 200, 100, 6, and 3 rpm speeds. Plastic viscosity and yield point are calculated using Equation 2 and Equation 3. Rotation is stopped for 10 seconds and the maximum initial dial reading at 3 rpm is recorded as gel strength. This is repeated for 10 and 30 minutes.

PV=R₆₀₀−R₃₀₀ Equation 0.10: Calculation for plastic viscosity in centipoise.

YP=R₃₀₀−PV Equation 0.11: Calculation for yield point in lb/100 ft².

All fluid loss measurements were taken in accordance with API RP 13B-1 and manufacturer operating instruction using an OFITE 4-Unit HTHP Filter Press. After adjusting the filter press thermocouples to 250° F., the pressurized collection cell is filled to 0.5 inches below the top. A filter paper is placed on top of the cell before installing the cap and tightening all screws and valve stems. The cell is inserted into the preheated jacket apparatus with the filter paper side down. After adjusting the top and bottom regulators to 100 psi, the top valve stem is opened to apply back-pressure to the fluid while heating. Once the cell has reached temperature, the top regulator was adjusted to 600 psi and the bottom valve opened to begin filtration at a differential pressure of 500 psi. After 30 minutes the bottom valve stem can be closed, and filtrate is collected from the condenser. To correct to the standard API filter size, the filtrate volume is doubled and recorded as the total fluid loss.

All lubricity measurements were taken in accordance with manufacturer operating instructions using an OFITE EP and Lubricity Tester. Prior to testing a set of drilling fluids, perform a calibration check on the OFITE lubricity meter by submerging the ring and block of the meter in deionized water while rotating at 60 rpm. Once the reading stabilizes, zero the torque on the meter. Using the torque arm, apply 150 inch-pounds of force for five minutes and record the stabilized torque reading. If the reading is not 34±2, repeat this process up to three times. If the issue remains after that, correct the calibration as outlined in the OFITE instruction manual. Once the reading for deionized water is within the acceptable range, record this value as the meter reading for deionized water. Calculate the correction factor (CF) using Equation 4.

$$Correction factor due to water reading .$$

$$CF = \frac{34}{Meter Reading for Water} \qquad Equation 0.12$$

After the calibration check, fill the sample container with the first drilling fluid and submerge the ring and block. Allow the readings to stabilize and zero the torque. Apply 150 inch-pounds of torque and record the reading after 5 minutes. Equation 5 shows the coefficient of friction (CoF) calculation using the torque wrench reading of 150 inch-pounds and torque shaft lever arm length of 1.5 inches. This can be corrected using Equation 6 and the correction factor from Equation 4. For an easier comparison of different studies and lubricants, a better measure is the percentage reduction of torque shown in Equation 7.

$$Coefficient of friction calculation for OFITE lubricity meter .$$

$$CoF = \frac{Torque Reading}{( \frac{150 in - lb}{1.5 in} )} = \frac{τ}{100 lb} \qquad Equation 0.13$$

$$Correction of coefficient of friction with correction factor .$$

$${CoF}_{corr} . = ( CoF ) ( CF ) \qquad Equation 0.14$$

$$Calculation of reduction in torque in percent .$$

$${\%}_{red} = \frac{CoF - {CoF}_{base}}{{CoF}_{base}} ( 100 ) \qquad Equation 0.15$$

All fluids tested in this section of experiments share the same base fluid with only lubricant type and concentration varying from sample to sample; however, there are slight fluctuations in the properties of the base fluid with each batch mixed. To reduce any skew of the data due to this, a control with no lubricant was run with each batch of testing to give a clear baseline. For an easier comparison among batches and tests, the results for each test are calculated as a percent change from the appropriate control sample. This also allows for a more straightforward method of comparing the results with literature data with which base fluid composition may differ.

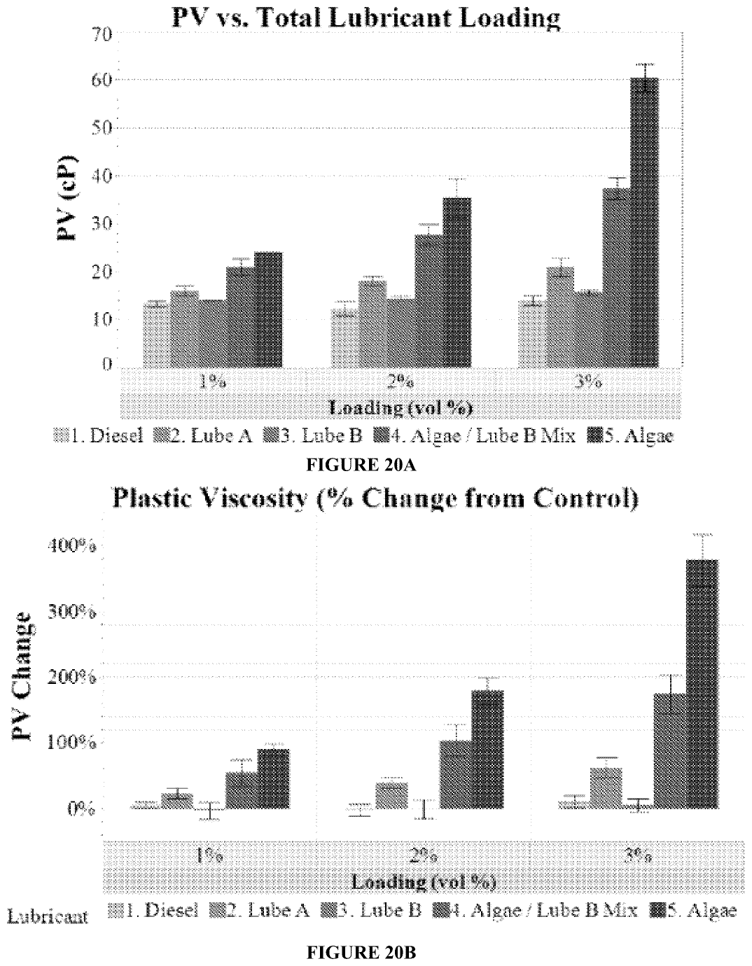

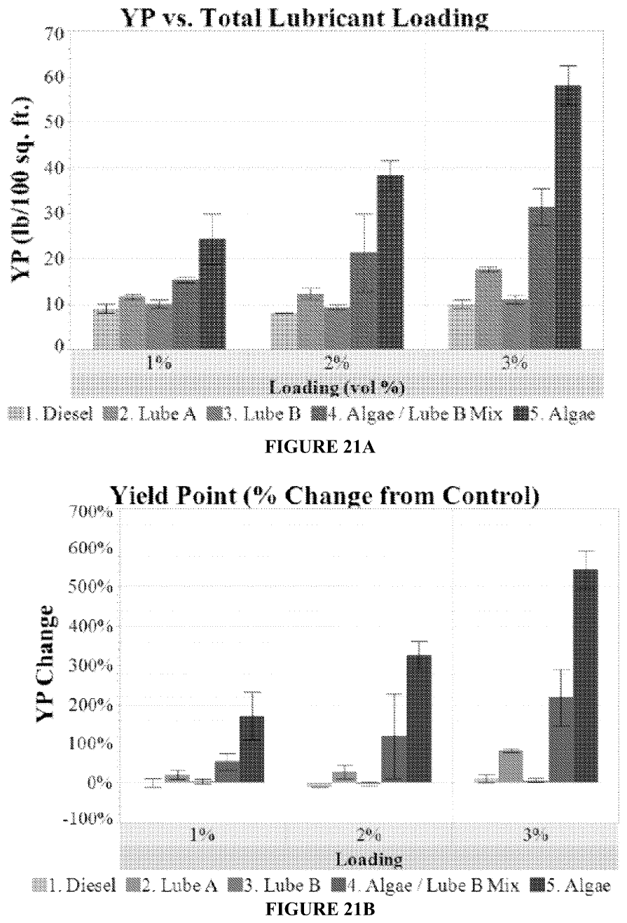

Bingham plastic is a common two-parameter model for non-Newtonian fluids and is the traditional method of fluid characterization in the field since the parameters can be quickly calculated without the use of a computer or complex algorithm. The plastic viscosity (PV) and yield point (YP) for each sample calculated using Equation 2 and Equation 3 are shown in FIG. 20 and FIG. 21. All values are an average of triplicate tests and the error bars are one (1) standard deviation from the mean. As seen in FIG. 20 and FIG. 21, the algae and algae/lube B samples had the largest effect on plastic viscosity and yield point with lube A also showing a lesser increase. At three (3) percent algae concentration the PV and YP increased from the control by nearly 380 percent and 540 percent, respectively. At the same total lubricant concentration, the half-and-half mixture of algae and Lube B only caused a 175 percent PV increase and a 220 percent YP increase.

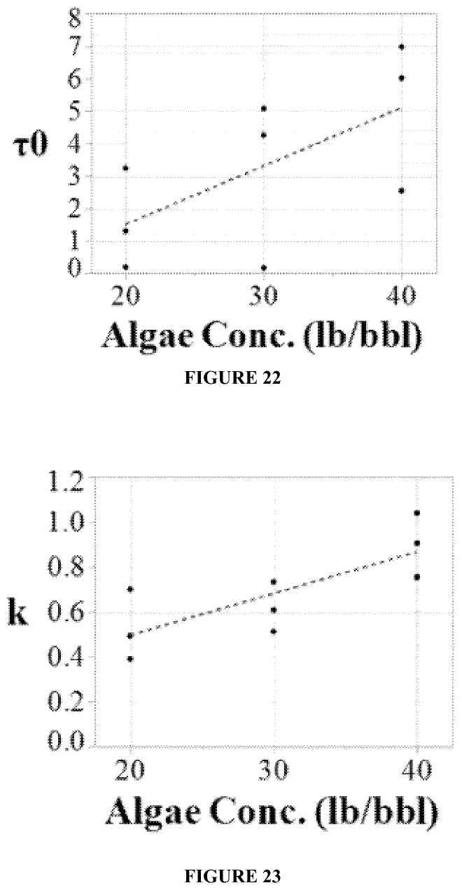

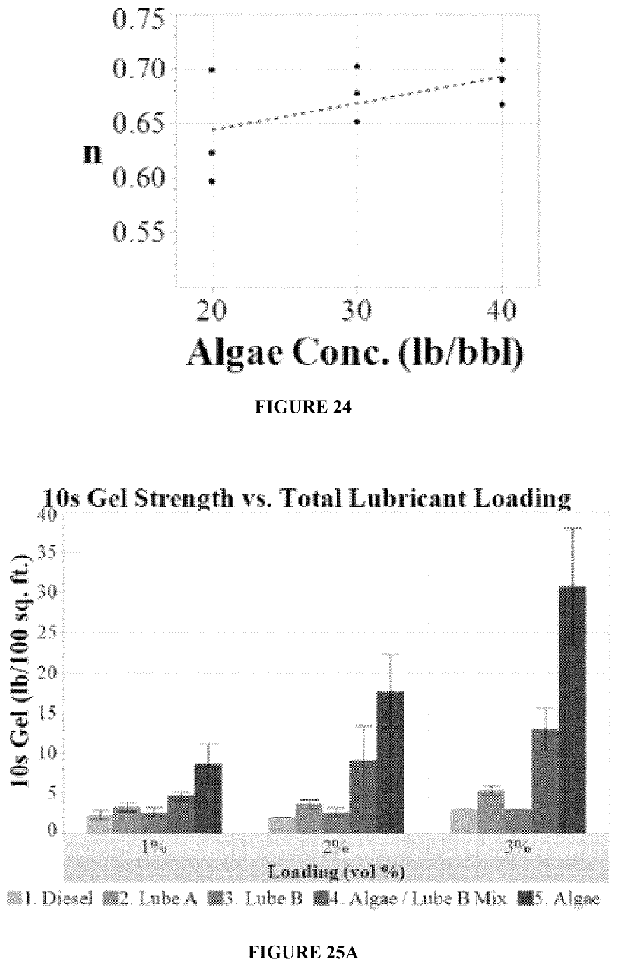

Though Bingham-plastic is commonly used for characterizing drilling fluid rheological properties, research has shown that Herschel-Bulkley, a three-parameter model for viscosity shown in Equation 8 is a better fit for the behavior of water-based bentonite drilling fluids. The rheology readings for all fluids with were fit to a curve using the Herschel-Bulkley model. The fitted model parameters versus algae and caustic concentration are shown in FIG. 22, FIG. 23, and FIG. 24.

τ=τ₀+k\dot{γ}ⁿ Equation 0.16: Shear stress as a function of shear rate using the Herschel-Bulkley model.

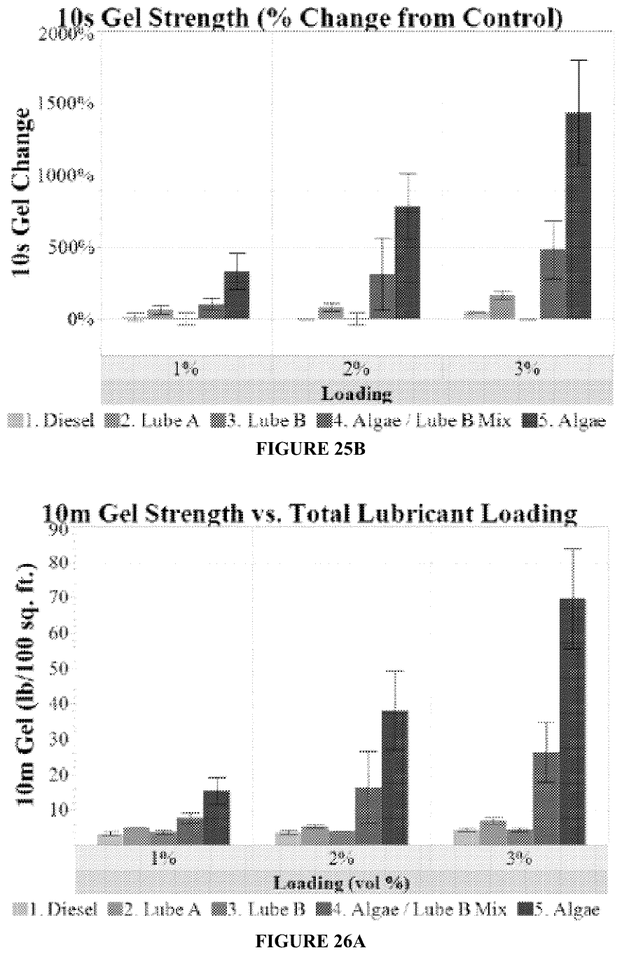

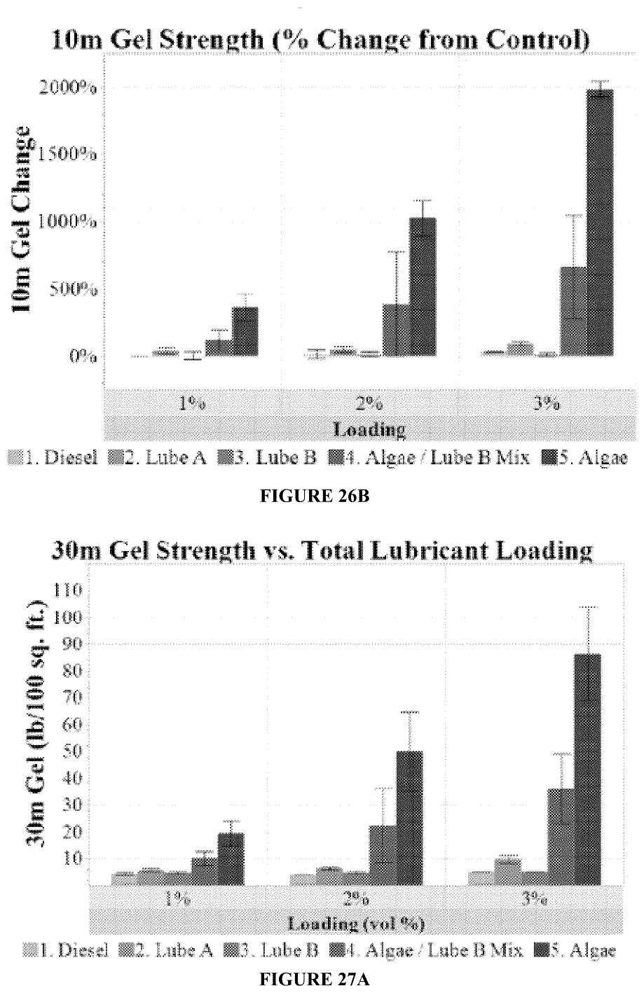

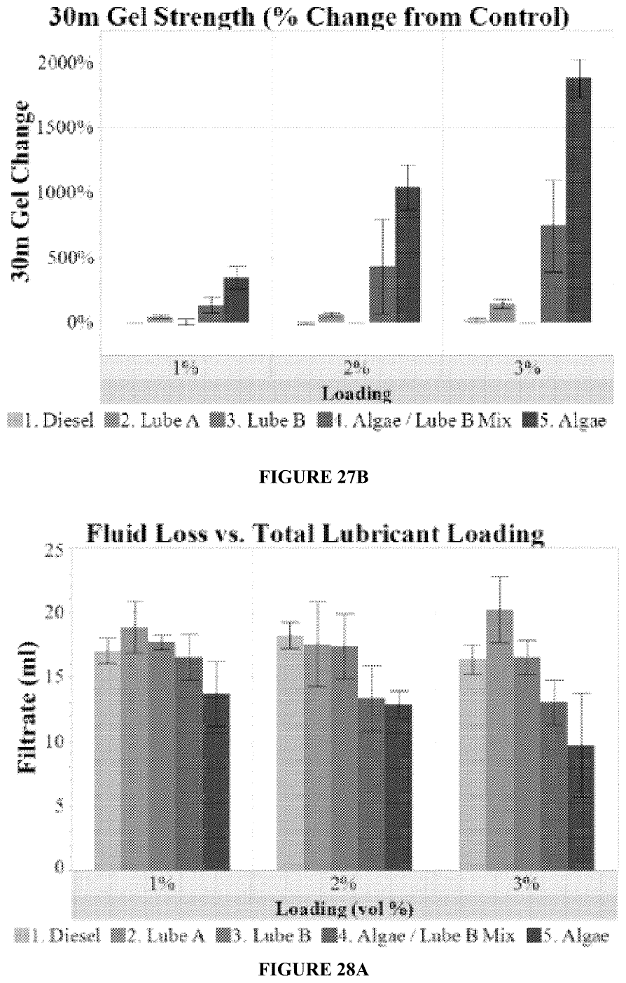

The gel strength is a measure of the ability of a fluid to maintain viscosity after a period of static rest. FIG. 25a,b, FIG. 26a,b, and FIG. 27a,b show the change in ten-second, ten-minute, and thirty-minute gel strength readings, respectively. All values are an average of triplicate tests and the error bars are one (1) standard deviation from the mean. Like the plastic viscosity and yield point, the gel strength reading is largely unaffected by diesel or Lube B alone; however, there is a slight impact from Lube A and a sizable increase from the algae and algae/Lube B samples. Because the control gel strength values were very low, the percentage increases may appear large. The algae ranged from an increase of about 300 percent at the lowest concentration to nearly 2000 percent at the highest, while the mixture with Lube B was around 100 percent and 750 percent at the same concentrations.

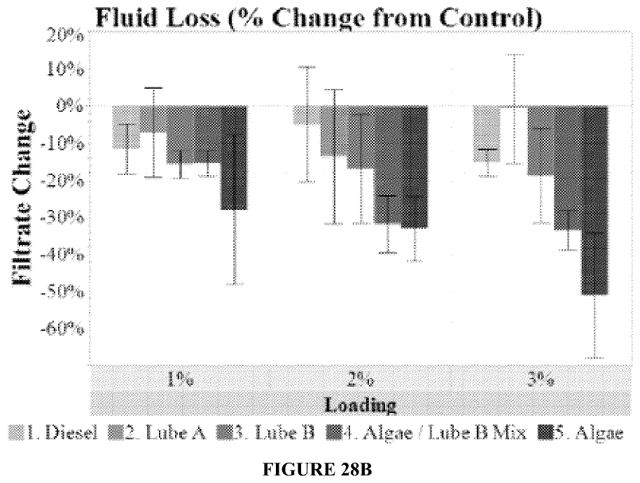

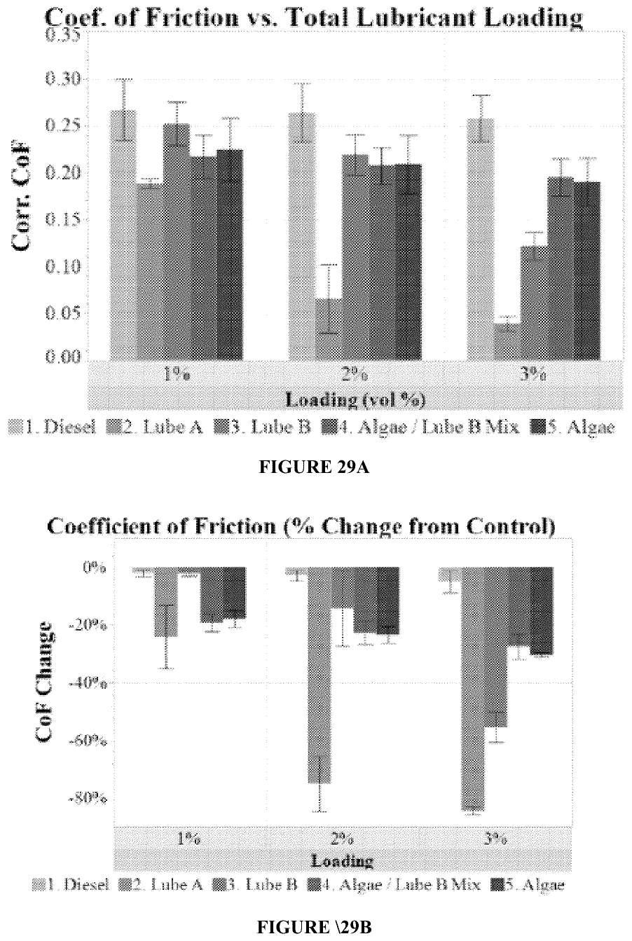

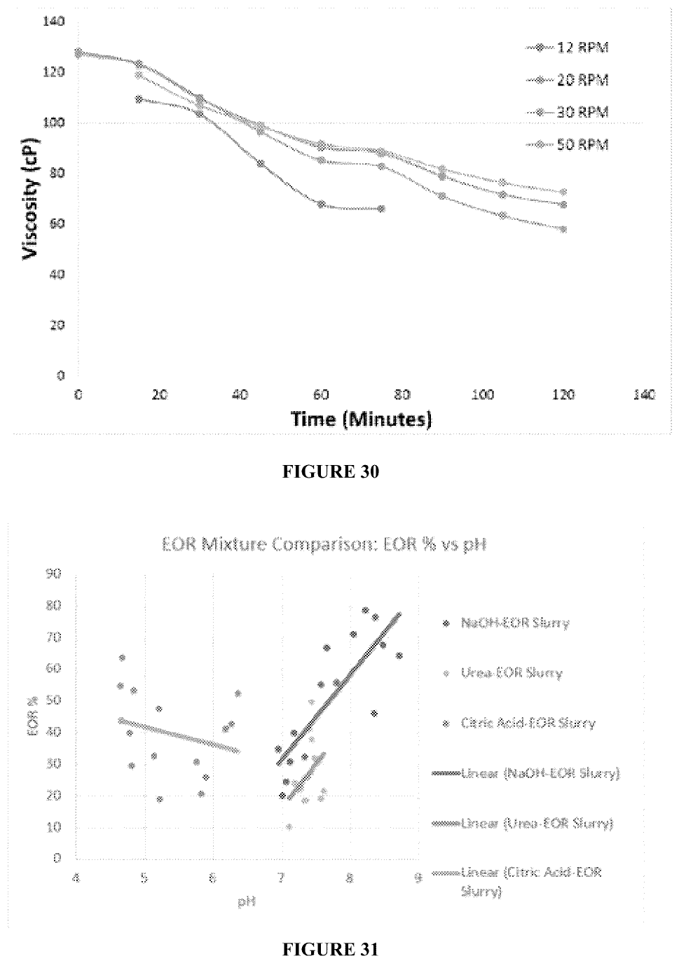

High pressure, high temperature filtrate volume is a laboratory measure of how well the sample can mitigate or reduce fluid losses into the pores of the formation. FIG. 28 shows the filtrate volume reported as fluid loss as described in the API procedure. All values are an average of triplicate tests and the error bars are one (1) standard deviation from the mean. The algae powder provided the greatest fluid loss reduction across all concentrations. Worth noting is that at two (2) percent total lubricant loading, the algae/lube B mixture exhibited nearly identical behavior to that of the algae alone even though it only contains half as much algae.

One of the most effective ways to measure the performance of a lubricant is the reduction in coefficient of friction or torque from a control. The values for this reduction are shown in FIG. 32 calculated using Equation 7. All values are an average of triplicate tests and the error bars are one (1) standard deviation from the mean. These results show that the commercial lubricant (Lube A) performed the best at all three concentrations. It is important to note, however, that the algae/Lube B mixture out performed Lube B at one (1) and two (2) percent concentrations and had very similar results to the algae powder alone despite having half as much of the substance.

As a drilling fluid additive and lubricant, the denatured biomass continued to show promising results. It improved the plastic viscosity, yield stress, fluid loss, and gel strength of the mixture more so than the other lubricants studied. 50:50 mixtures of Lube B with denatured biomass also showed promising results. In regard to lubricity as assessed by coefficient of friction reduction, the denatured biomass did not perform as well as Lube A which is environmentally hazardous. However, the denatured biomass outperformed Lube B, especially at lower loadings, which indicates that it is highly competitive in the “environmentally friendly” and EPA-approved class of drilling fluid additives. It should also be noted that combinations of these additives may be of strategic value. For instance, the 50:50 mixture of Lube B and our biomass additive had a significantly lower plastic viscosity but had a similarly lubricating effect as evidenced by the CoF reduction, so this may be a good solution where a lower viscosity is desired but more lubrication is needed.

This part of the study investigates interfacial and rheological properties of novel, Spirulina-based fluids developed for potential use in enhanced oil recovery (EOR) applications. The purpose of this investigation is to determine the driving mechanism(s) behind the EOR performance of these materials after a previous study concluded that the well treatment compositions are a promising technology with up to 96% recovery of the original oil in place (OOIP). It was then hypothesized that the EOR mechanism arose from the interfacial energy effects and/or the rheological properties. In this study, Arthrospira platensis (Spirulina) biomass is modified by different denaturants (sodium hydroxide, citric acid, and urea) and tested for a wide range of interfacial and rheological properties. The interfacial properties include surface tension, interfacial tension with hydraulic oil, contact angle against silicon dioxide, and work of adhesion between the fluid and the silicon dioxide surface. The rheology focuses on determining fluid viscoelastic properties and flow behaviors. Additionally, this study compares and correlates the results amongst each other, which also includes pH, EOR performance, and denaturant concentration.

The EOR performance of these fluids strongly correlates with the increased viscosity, reduced interfacial tension (IFT), and increased wettability in the well treatment compositions. All of the investigated EOR solutions reduced the IFT. Most of the EOR solutions were found to be mostly shear-thinning with shear-thickening approximately in the 2,000-5,000 s⁻¹ range. From the results, it is concluded that the performance of the different types of well treatment compositions is based on different mechanisms. Sodium hydroxide-based fluids are driven by increased low-shear viscosity, reduced IFT, alkaline pH shift, and improved wettability. The citric acid-based fluids do not exhibit high viscosity values in the low shear range, which indicates that their EOR performance seems to be driven by reduced IFT, acidic pH shift, improved wettability, and increased fluid rigidity by the generation of crosslinks in the macromolecular structure. Urea-based fluids did not exhibit improved viscosity, which means that their modest EOR performance is likely driven by reduced IFT and improved wettability. While a shear-thickening rheology is often considered to be a mechanism for improving EOR, it is concluded that the shear-thickening effect is not a dominant mechanism for these fluids, especially since the highest performing EOR fluid (highest NaOH concentration) did not display shear thickening.

The decrease of oilfield exploration projects combined with the production decline in mature fields [1,2] has resulted in an increase in demand for enhanced the oil recovery technologies for production operations. It is estimated that only approximately-one-third to two-fifths of original oil in place (OOIP) is produced via primary and secondary oil recovery methods [3]. Polymer injection is a popular form of chemical injection for tertiary oil recovery. As resource demand and literature of petroleum-related applications continues to grow, so does the interest in chemical flooding EOR practices, due to their wide variety of mechanisms and their versatility in reservoir conditions [4-7]. Polymer injection is comprised of incorporating high-molecular-weight, water-soluble polymers into injection water, which will increase the fluid viscosity and reduce formation permeability. Increasing the viscosity and reducing water-phase permeability will reduce the mobility ratio of the flooding process, which leads to an overall increase in the efficiency of macroscopic displacement of petroleum [8].

In pursuit of low-cost, renewable, and eco-friendly EOR materials, this study focuses on determining the driving mechanism(s) of the EOR performance of chemically modified Arthrospira platensis (Spirulina) biomass, which is seen as potentially suitable candidate to create EOR polymeric bio-sourced fluids based on a previous study [9]. Spirulina has applications in biofuel production, wastewater treatment, and nutraceutical production, all of which yield a Spirulina-based biomass as a waste product of those processes. In addition to being an effective EOR agent, Spirulina-based biomass has the potential to make the aforementioned bioprocesses more profitable or commercially viable. The aim of this article is to study the interfacial and rheological properties of these novel, Spirulina-based well treatment compositions using food-grade Spirulina biomass as a water-soluble base material to be modified by three different denaturants: 1 M sodium hydroxide, 1 M urea, and 1 M citric acid. Surface tension, contact angle, volumetric analysis, interfacial tension between the EOR fluid and oil, work of adhesion, pH, visco-elastic moduli, and flow curve data are investigated to determine correlations and provide insight regarding EOR performance mechanisms of these fluids.

This work looks at three different protein denaturants which utilize different denaturation mechanisms. Sodium hydroxide was used to see the effect of an alkaline denaturant and is a common industrial byproduct. Citric acid represents an acidic denaturant and has the advantage of being bio-sourced. Urea can also be bio-sourced and represents a near-neutral, chaotropic denaturant.

While this is the first paper to investigate the mechanisms of the EOR effect from denatured algal biomass, there have been a variety of other EOR agents proposed from other biomass sources. One more common proposed technology in recent literature is microbial EOR (MEOR) [10], which is centered around living microbes [11,12] or their byproducts [13], which is different from this study focused on denatured biomass which is dead or inactive. Other novel biomass studies for EOR include using sawdust as a thermal EOR agent to reduce the viscosity of extra heavy oil and utilizing a fertilizer solution comprised mostly of beer yeast residue as a chemical surfactant alternative for EOR [15]. Some studies have extracted biosurfactants from bacterial strains or biomasses such as Shewanella oneidensis [16], Aeromonas hydrophilia [17], Pseudomonas aeruginosa [18], and Bacillus mojavensis [16]. Other studies derived surfactants/extracts from plant material, such as red onion skin [19], red beets [20], Malania oleifera and Lunaria annua seeds [21], sunflowers [22], or from coconuts, corn, potatoes, and wheat [18]. Waste-derived materials for EOR are another new area of research for EOR processes, such as waste cooking oils or paper industry black liquor [24].

The hypotheses of this study were that the EOR performance was caused by three driving mechanisms: the increase the fluid work of adhesion with sandstone, reducing fluid—oil IFT, and/or by the fluids having a dilatant rheology. Work of adhesion represents the energy necessary to separate two phases in contact with each other, where a fluid with high work of adhesion values with sandstone/SiO2 would likely displace hydrocarbons that are in contact with sandstone surfaces. Reducing the IFT of the fluid-oil interface would mean that the hydrocarbons are able to generate more stable emulsified phases with the injection fluid, thus increasing the amount of oil produced from the EOR processes. Dilatant (shear-thickening) fluids are desired for EOR injection because they reduce the channeling through porous media by slowing the fast-moving fluid in the larger pores, which can increase the amount of oil produced by allowing for a more even sweep by the well treatment composition.

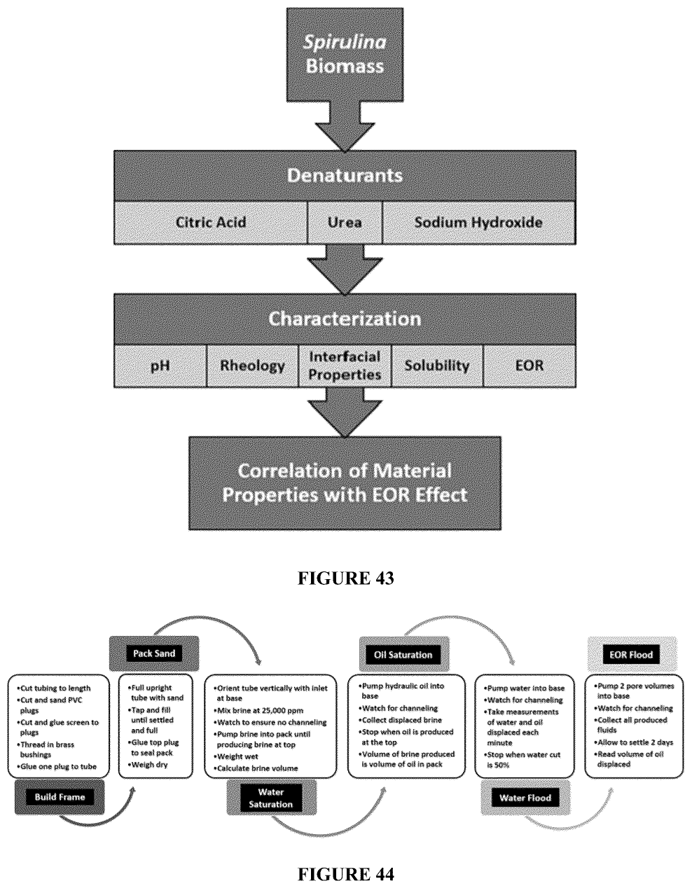

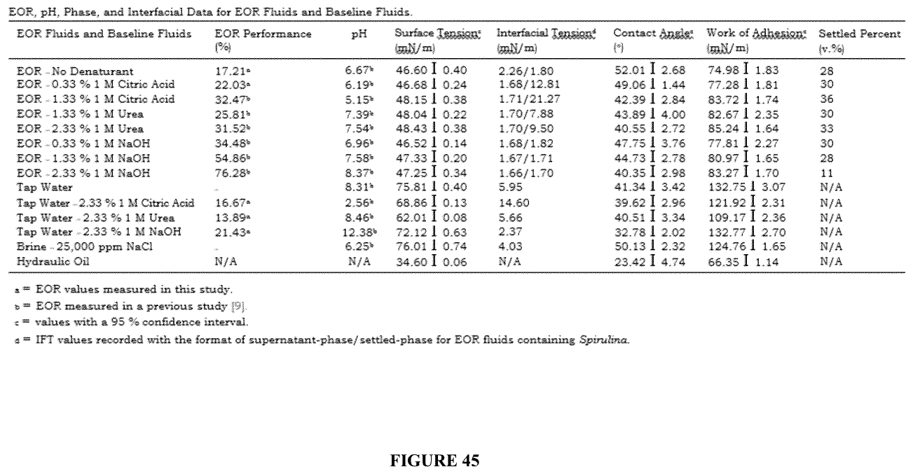

An overview of the work is illustrated in FIG. 43. Spirulina blue-green cyanobacteria biomass was purchased from Acetar Bio-Tech Inc. (Xi'an, China) with a protein content of 62.2 wt % and 6.8% mass loss on drying. The three denaturants used in this study are 1 M citric acid, 1 M urea, and 1 M sodium hydroxide with their base chemical reagents purchased from Flinn Scientific (Batavia, IL, USA). Mobil DTE 26, batch #70272502, hydraulic oil was obtained from Exxon Mobile. A quartz crystal was obtained from the School of Geo-sciences of the University of Louisiana at Lafayette.

To make samples comparable to a previous EOR study [9] and minimize material consumption, the size and number of well treatment compositions are scaled down to 100 mL of tap water with a constant 9.33 g of biomass and 1 M denaturant volumes of 0.33 mL, 1.33 mL, and 2.33 mL. Each sample is mixed for 1 h. It was observed after 2 h of settling time that the prepared EOR fluid samples are comprised of a transparent, supernatant solution and settled-particulate components. 50 mL of samples were centrifuged at 180 G-Force for 15 min in order to obtain a clear separation between the components. After centrifuging, the components are separated into separate containers and centrifuged for another 15 min under 180 G-Force to ensure substance purity.

Surface tension and interfacial tension characterizations were carried out using a KSV Sigma 702 Force Tensiometer using the platinum-iridium Du Noüy Ring method. A minimum of three duplicate batches for each of the well treatment compositions were assessed with at least 15 repeated data points after the interfaces stabilized. For interfacial tension measurements, both the supernatant phase and settled components of each mixture were tested at an interface with the hydraulic oil. The fluids, interface, and Du Noüy ring are allowed to sit idle for 30 min to ensure that a stable interface has been formed between the EOR fluid component and the hydraulic oil interface.

In preparation for contact angle measurements, a quartz crystal is cleaned with water, followed by 99% isopropanol alcohol, and then allowed to dry for 48 h. While the EOR fluid is mixing, the quartz crystal is continuously heated and maintained for 1 hr at 125±5° F. (−49-54° C.) and positioned under a constant airflow to preserve a clean testing surface. The temperature is monitored by utilizing an Etekcity Lasergrip1022 Infrared Thermometer. Once the heating treatment is finalized, the crystal is removed and allowed to return to 75±3° F. (−22-25° C.) for the contact angle tests. The quartz surface is then horizontally positioned on the KRUSS Advance Drop Shape Analyzer stage. Using the sessile drop method, small droplets of EOR fluid are carefully applied to the surface of the crystal with a blunt-tip syringe and the contact angle values are recorded with the ADVANCE software. One sample of each of the well treatment compositions was mixed and tested for contact angle values with 20 individual droplets. Contact angle data for baseline fluids such as tap water, brine (25,000 ppm NaCl), tap water with 2.33% of each 1 M denaturant, and hydraulic oil were also obtained.

Young's equation is described in the equations below and the work of adhesion is described by Equation 0.18 and Equation 0.19 [25]. _{Wadh} is the work of adhesion, E is the surface tension value, and 9 is the contact angle. The subscripts of S, L, and A depict the interfaces for

solids, liquids, and air, respectively [25]. For determining the mean work of adhesion values, the mean values of contact angle and surface tension are used.

ES/A=ES/L +EL/A cos(9) Equation 0.17

Wadh=ES/A+EL/A [symbol] ES/L Equation 0.18

Wadh=EL/A[1+cos(9)] Equation 0.19

Oscillatory and rotational rheology characterizations were carried out on an Anton Paar Modular Compact Rheometer 302 (MCR-302) using the parallel plate method and Rheoplus software. The stainless steel, parallel-plate attachment had a diameter of 25 mm. The plate gap was set to 0.1 mm. All measurements were performed at 20° C. and atmospheric pressure.

The oscillatory linear viscoelastic measurements were comprised of the amplitude sweep and frequency sweep tests. The amplitude sweep measurements were taken in the strain range from 0.1 to 1000% at a constant angular frequency of 10 rad/s. Frequency sweep measurements were performed in the range from 0.1 to 100 rad/s at a constant strain value of 0.5%, which was determined from the amplitude sweep results. The amplitude sweep is tested in order to observe the linear viscoelastic range (LVER), storage moduli, loss moduli, and to be able to choose a constant strain value that does not destroy the material's structure during frequency sweep experiments, which are also used to evaluate the storage and loss moduli of the fluids.

The flow curves with controlled shear rate (CSR) are measured in the range increasing from 0 to 20,000 s⁻¹. Both viscosity and shear stress flow curves are measured in order to characterize the differences in viscosity values of the well treatment compositions and to observe their non-Newtonian behaviors.

The thixotropy experiments are used to evaluate the time it takes to rebuild the fluid structures after experiencing high-shear loads. The first thixotropy interval and third thixotropy interval both have a constant shear rate of 10 s⁻¹, which is to simulate a natural flow through porous media of reservoir formations [26]. The second thixotropy interval is set to induce a higher shear rate of 200 s⁻¹ in order to break the fluid structure and allow the structure to rebuild during the third interval.

Sand-pack flooding experiments were conducted in this study for fluids containing Spirulina without denaturant and the denaturant solutions themselves. This data is crucial for correlating the different data sets in this study. In this study and a previous study [9], hydraulic oil is used because it has physical properties comparable to “dead” crude oil and is considered to be an incompressible fluid since no vapors are being released. This allows for multiple experiments over time to be compared without the variance caused by the sampling of crude oil or its variance over time. Other studies in recent literature have used similar types of petroleum-based products for EOR-related applications, such as hydraulic oil or mobil oil [27], crude oil/kerosene mixtures [28], and light mineral oil [29].

A flowchart of sand-pack preparation for EOR flooding is detailed in FIG. 44, which is the same preparation used in a previous study [9]. Sand was compacted into clear acrylic tubing capped with steel mesh and brass bushings. These enclosed tubes of sand, denoted as sand packs, were saturated with a 25,000 ppm NaCl brine, then with hydraulic oil, then flooded with water to reach a 50% critical water cut, which is typically between 50 and 99% depending on operating conditions and the crude oil economy. The sand packs were then finally pumped with two pore volumes of the relevant EOR fluid at flow rates up to 25 mL/min and the produced fluids were collected in graduated cylinders in which the fluids were then separated via gravity and centrifuging for calculation purposes. To reduce fracturing of the formation, no fluid flow was performed above 25 psi. This group's previous study [9] offers a more detailed explanation of the sand-pack flooding experiments. Measurements for oil recovery were a visual reading of the oil level in a graduated cylinder, plus 66.73% of the volume of the agglomeration, which has been determined experimentally by centrifugation.

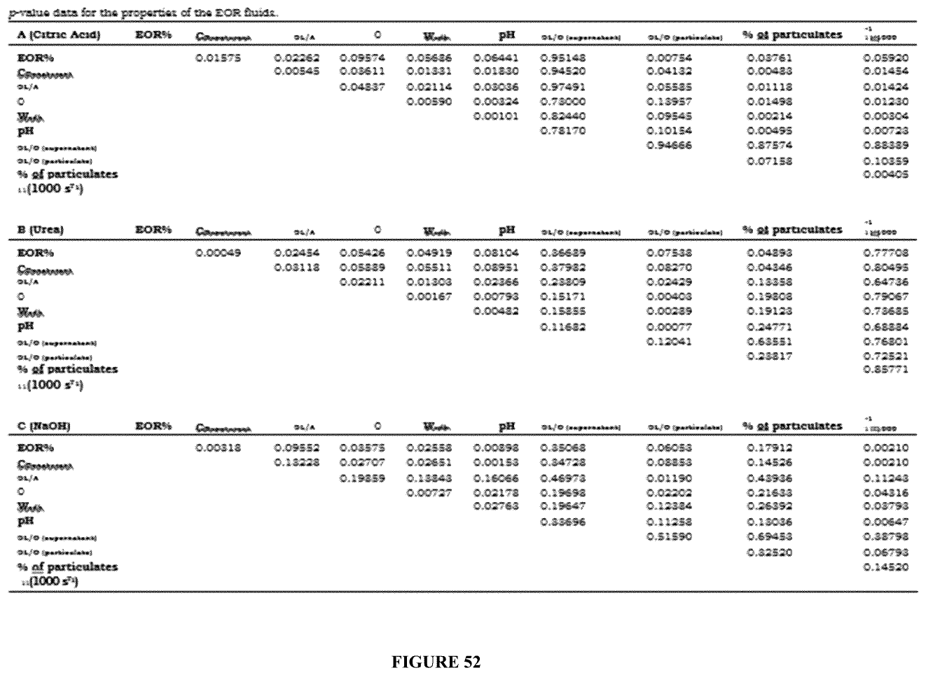

The pH values for the well treatment compositions are listed in column 3 of Table 6 shown in FIG. 52. The well treatment compositions all exhibited changes in pH as denaturant concentration increased. The Spirulina biomass had an acidic effect on the tap water, which indicates that the biomass is anionic in aqueous solutions. The pH shift after adding biomass to the denaturants indicates that there are significant chemical interactions between them. In general, it was observed that the Spirulina had a sort of pH buffering effect in that it moved the acidic and alkaline EOR denaturants toward a neutral pH.

The surface tension results of the statistical analysis are listed in column 4 of Table 6 shown in FIG. 52. All of the well treatment compositions displayed increases in surface tension as the concentration of 1 M denaturants increase. The only exception to this observation is the EOR fluid with the 2.33% concentration of 1 M sodium hydroxide, which experiences a slight decrease in surface tension compared to the EOR fluid with 1.33% 1 M sodium hydroxide.

The tap water and brine average surface tension values were 75.81 and 76.01 mN·m⁻¹, respectively, which agrees with previous observations of salinity increasing the surface tension [30]. The addition of the Spirulina biomass had a surfactant effect, decreasing the fluid surface tension values significantly.

The denaturants decreased the surface the interfacial tension values of both base fluids as well as the EOR fluid supernatant phase and settled phases are listed in column 5 of Table 6 shown in FIG. 52. Both the citric acid-based and urea-based EOR fluid components displayed increases in interfacial tension as the concentration of denaturants increase while the sodium hydroxide-based EOR fluid components displayed IFT reduction as the concentration increased. Overall, the addition of the Spirulina to tap water reduced the IFT value of the fluid, and denaturant influence in the well treatment compositions reduced the IFT values further. The exceptions to these observations are the settled components of the urea-based and citric acid-based well treatment compositions.

The contact angle results of the statistical analysis are listed in column 6 of Table 6 shown in FIG. 52. Against a silica surface, the EOR fluid without denaturant and the brine yield the two highest contact angle results, while the hydraulic oil yields the lowest contact angle. The contact angle results show that increasing concentration of all three denaturants will increase the wettability of the well treatment compositions on quartz surfaces. The contact angle for tap water was lowered when denaturants were introduced. The lowest of the three denaturant solutions is the tap water that contains 2.33% 1 M sodium hydroxide, which reduced the tap water contact angle by 8.56°. This observation indicates that introducing sodium hydroxide to well treatment compositions increases wettability as expected from literature when the pH deviates from neutral [31,32], However, the well treatment compositions with concentrations of sodium hydroxide did not substantially increase the wettability much more than the other well treatment compositions. These wettability observations regarding sodium hydroxide indicate that higher concentrations of sodium hydroxide may be beneficial for enhancing wetta-bility to enhance EOR. Alkaline environments alter the wettability of sandstone surfaces, which may result in changing fluids that are initially adhered to the sandstone. Silicon dioxide (quartz) has a negative surface charge and clays have a positive surface charge. Alkaline environments increase the negative charge of the surfaces, which promotes an increased negative charge on silica and an alteration from positive to negative charge on clays. It is clear from Table 6 shown in FIG. 52, that the denaturants and the biomass affected the contact angle with the denatured biomass having a contact angle between the biomass and denaturants alone. Identification of specific adsorptive groups will be a subject for future work, but the denaturants could also be modifying the biomass to affect the contact angle results. For instance, if the denaturants allow for the activation of organic acids, they are known to reduce contact angle to sandstone and carbonate rocks [33].

The work of adhesion values for the well treatment compositions are listed in column 7 of Table 6 shown in FIG. 52. The well treatment compositions all exhibited increases in adhesive energy as denaturant concentration increased. Addition of the Spirulina biomass decreased the adhesion value significantly, while the denaturants seemed to increase it. The adhesive energy on a quartz surface for the well treatment compositions are all less than that of the various tap water solutions and the brine. However, all fluids that were measured have higher adhesive energy values on silica surfaces than the hydraulic oil does, which is known to be a significant aid when implementing wettability-altering solutions in oil-wet and intermediate-wet reservoirs. Since the work of adhesion for the well treatment compositions is less than that of water, the adhesive energy of the well treatment compositions is unlikely to be the primary driving mechanism in their chemical flooding performances.

There are significant relationships between denaturant concentration and the interfacial properties of surface tension and IFT, especially for the solutions containing sodium hydroxide and citric acid. Furthermore, it is known that Spirulina contains water-soluble globular proteins, which have stabilization properties for fluid interfaces and emulsions [34]. The Spirulina biomass contributed a 62-69% reduction in fluid-oil IFT, and achieving values as low as 1.66 mN/m when sodium hydroxide is added. The recorded IFT values are not close to 10⁻ 3-10−2 mN/m in value, which is not considered to be “ultra-low IFT” [35-37]. This indicates that IFT reduction of these fluids cannot be considered to be the sole EOR driving force for EOR performance, but the significant reduction is highly likely to still have a positive contribution to increasing their EOR performance.

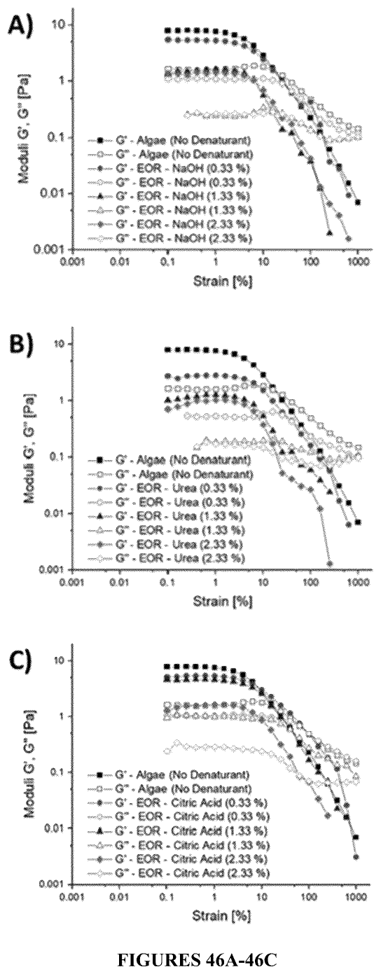

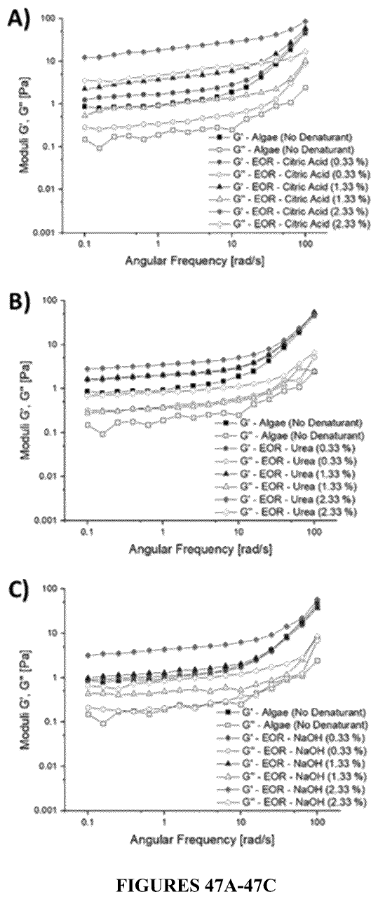

As shown in FIG. 46, all well treatment compositions were tested in a frequency-controlled strain sweep at a constant temperature of 20° C. and a constant frequency of 10 rad·s⁻¹. Within the LVER, all well treatment compositions exhibited a storage modulus that was greater than the loss modulus, which indicates that their viscoelastic properties are elasticity dominated. All the well treatment compositions displayed decreases in moduli by an order of magnitude as their denaturant concentration increased.

Furthermore, the crossover points of the well treatment compositions deviated as result of incorporating certain denaturants, where citric acid-based well treatment compositions displayed slight deviations in the crossover point to the right and both urea- and sodium hydroxide-based well treatment compositions displayed left-side deviations of the crossover point. The structure of the well treatment compositions did not destabilize until reaching at least 1% strain values. Therefore, a viscoelastic properties are structurally elastic. All the well treatment compositions displayed increases in moduli as the concentration of denaturant increased. The highest storage modulus achieved is the EOR fluid with 2.33% of 1 M citric acid.

The modulus magnitudes for the Spirulina-based well treatment compositions are comparable to industry standard well treatment compositions comprised of either xanthan gum or hydrolyzed polyacrylamide. A 1 wt % xanthan gum EOR fluid, which has storage moduli being greater than loss moduli (G′>G″), yields an approximate storage modulus value of 1 Pa within the LVER of the amplitude sweep [38] and a 1 Pa storage modulus at 1 rad/s for frequency sweep [39]. However, a 0.5 wt % HPAM EOR fluid exhibits a modulus value around 0.2 Pa within the LVER [40] and a 4.0 wt % HPAM fluid exhibits around 8 Pa [41]. Additionally for 1 rad/s, a 0.5 wt % HPAM will exhibit a modulus around 0.4 Pa [40] and the 4.0 wt % HPAM fluid will yield an approximate 10 Pa modulus value [41]. With these literature values, we can say that the Spirulina-based well treatment compositions have adequate oscillatory properties, specifically that the amplitude sweep moduli are within the LVER and frequency sweep moduli at 1 rad/s fall between approximate ranges of 1-8 Pa and 1-20 Pa, respectively.

The EOR fluid component analysis of all EOR fluid supernatant and settled phases is listed in column 8 of Table 6 shown in FIG. 52. Both the citric acid-based and urea-based well treatment compositions generated larger amounts of settled components as the denaturant concentrations increased. However, the sodium hydroxide-based well treatment compositions reduce the amount of the settled components as the denaturant concentration increased. The settled components for the citric acid-based well treatment compositions were significantly more viscous/gel-like during extraction for the centrifuge methods and IFT analysis than the other two denaturant-based well treatment compositions.

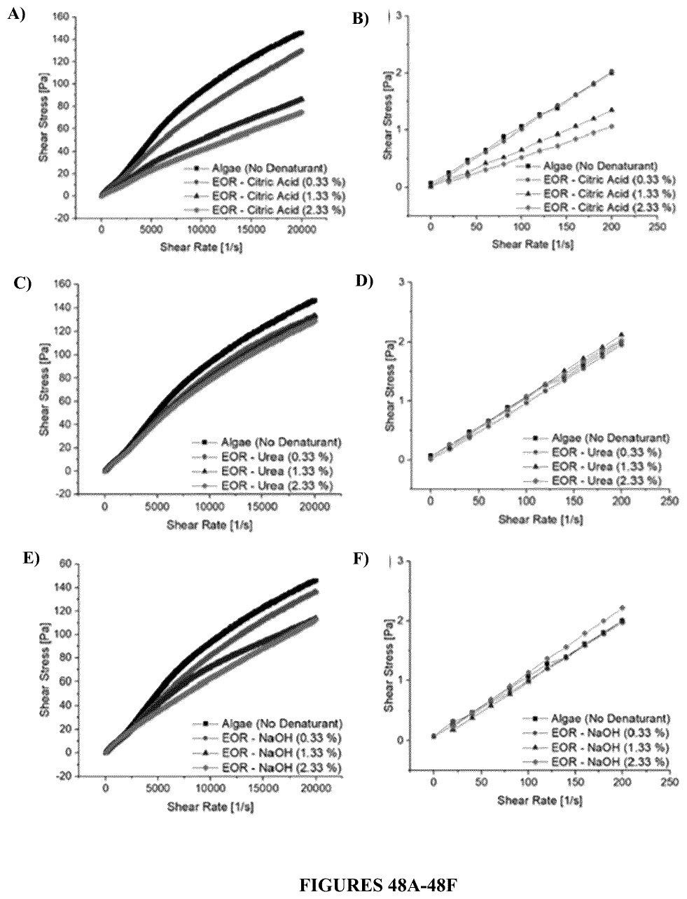

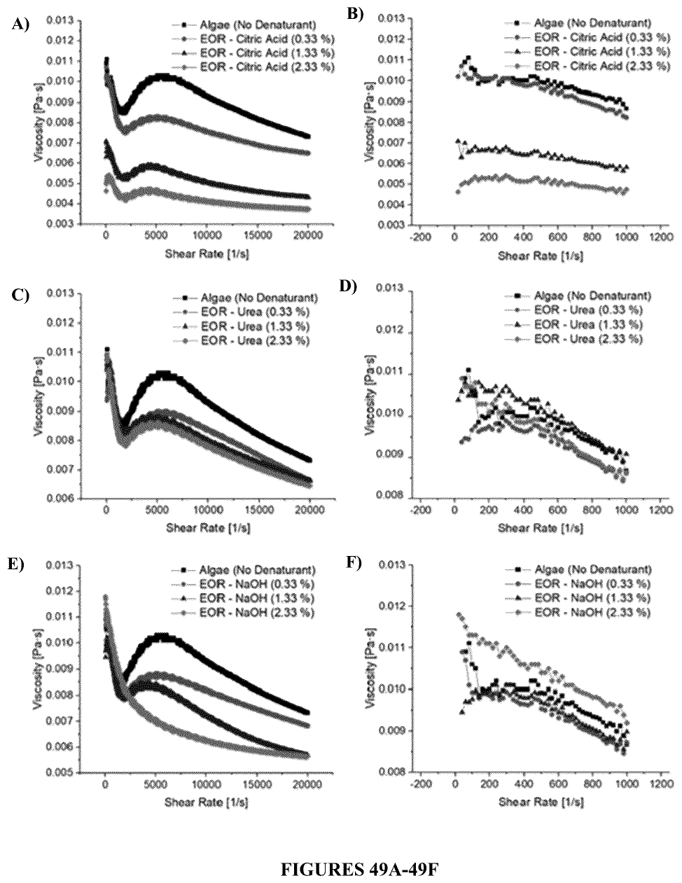

As shown in FIGS. 48 and 49, all well treatment compositions were tested in a controlled-shear rate flow curve at a constant temperature of 20° C. All of the well treatment compositions exhibited shear-thinning behavior in the approximate 0-2,000 [symbol] 1 range of shear rate that was investigated accompanied with reductions in viscosity as shear rate increases. FIG. 49 C′ shows that the sodium hydroxide-based well treatment compositions were found to yield higher viscosity values than the other well treatment compositions in this low shear range when high concentrations of denaturant is used. On the other hand, FIGS. 49A and 49A′ depict that the viscosity of the citric acid-based well treatment compositions decreased significantly in this shear range as the concentration of denaturant increases. well treatment compositions containing urea displayed no distinct concentration-related trend or change in viscosity at this shear range. With the exception of the EOR fluid containing 2.33% of 1 M sodium hydroxide, all of the well treatment compositions exhibit shear-thickening after approximately 2,000, which is later followed by another region of shear-thinning until a final shear rate of 20,000 s⁻¹ was reached. The approximate shear-thickening intervals are 2,000-7,000 s⁻¹, 2,000-4,500 s⁻¹, 2,000-5,200 s⁻¹, 2,000-4,000 s⁻¹, for the undenatured well treatment compositions, citric acid-based well treatment compositions, urea-based well treatment compositions, and sodium hydroxide-based well treatment compositions, respectively.

Some of the well treatment compositions, most notably the undenatured EOR fluid, experience a profound increase in fluid viscosity within shear-thickening interval. The undenatured well treatment compositions exhibit a shear-thickening region which is understood to be a direct result of the insoluble Spirulina soft-body colloids that are suspended in the liquid. Additionally, it is determined that the intensity of the shear-thickening regime is directly related to the amount of these soft-body colloids within the different well treatment compositions. Furthermore, it is observed that increasing denaturant concentration reduces the magnitude of this increased viscosity region, which is observed to be completely absent for the EOR fluid containing 2.33% 1 M sodium hydroxide. In the shear ranges greater than 2,000 _{s_}1, the overall viscosity values for each EOR fluid in these two regions are consistently reduced as denaturant concentration increases.

In comparison to the standard xanthan gum or hydrolyzed poly-acrylamide, the viscosity values of the Spirulina-based well treatment compositions are significant. In literature, a 1 wt % xanthan gum EOR fluid is known to exhibit a viscosity of approximately 0.015 Pa·s at 100 s⁻¹ [38,39,42-44], while 1 wt % HPAM well treatment compositions have been recorded to have viscosity values ranging from 0.005 to 0.015 Pa·s at 100 s⁻¹, depending on the study [44,45]. From these literature results and given that 0.0113 Pa·s was the highest viscosity achieved at 100 s⁻¹ for these Spirulina-based well treatment compositions, we can say that these algal well treatment compositions are comparable to HPAM and xanthan gum EOR standards.

In recent literature, many studies evaluate the practical rheological performance of well treatment compositions at shear rates no higher than −1,000 s⁻¹, [46-49] as it is discussed that higher shear rates are thought to not be prevalent throughout the petroleum reservoir's porous media [50]. Thus, while not applicable in terms of petroleum reservoir flow through porous media, it is seen that all of the well treatment compositions exhibit shear-thinning behavior until −2,000 s⁻¹, where the insoluble colloids begin to interact with each other and increase the viscosity in terms of shear-thickening. More specifically, the exhibited shear-thickening behaviors are

attributed to the formation of hydroclusters [51] amongst the colloids and the generation of friction between them and other materials within the well treatment compositions. It is understood that when a certain level of critical shear is reached for each respective EOR fluid, the present hydroclusters will elastically deform or even experience full breakage, in which either scenario would lead to another shear-thinning regime [51].

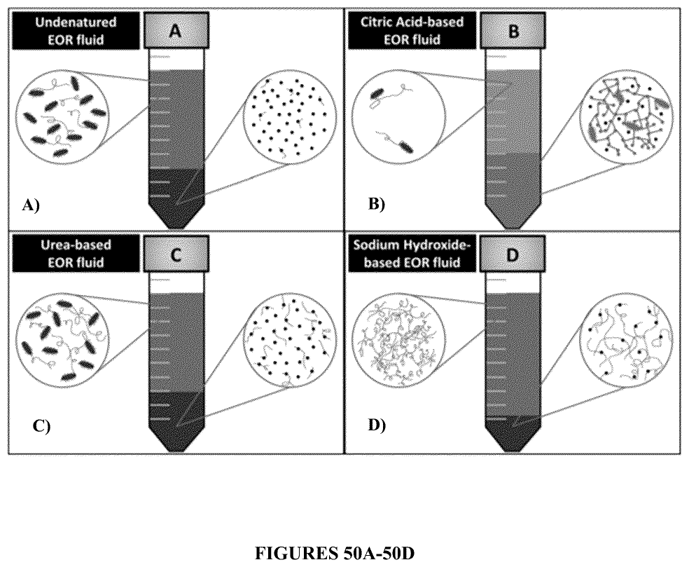

As depicted in FIG. 50, the denaturants interact with the insoluble colloidal particles, and thereby reduce the amount of soft-body colloids in the supernatant by solubilization through denaturation or by promoting colloidal interactions with the polymeric network to facilitate flocculation and settling. From noting the amount of the solids-containing settled phase, sodium hydroxide is causing a large degree of hydrolysis and solubilization, while the citric acid is resulting in the flocculation and settling of the biomass. In the top layer (supernatant phase) in the centrifuge tubes is thought to contain the water-soluble globular proteins of Spirulina with different levels of partial denaturation amongst the different well treatment compositions. It has been stated in literature that adding Spirulina proteins to water can result in partial protein denaturation [52], where the extent of partial denaturation would increase with shear-induced mixing [53]. The bottom layer (settled phase) is understood to contain insoluble Spirulina components which, among other solid components, may also include proteins that became insoluble due to denaturation or crosslinking. The insoluble proteins in the “settled phase” are

likely to be fibrous proteins [54]. The different layer volumes displayed in the figure are comparable to the data in Table 6 shown in FIG. 52. The color change illustrated in FIG. 50B reflects the color change observed in the experiment, where the supernatant phase had a distinct blue color and the settled phase turned to a lighter green color.

In the citric acid-based well treatment compositions, the shear-thickening region was observed within the shear interval of 2,000-4,500. Compared to the undenatured EOR fluid, the shear thickening regime has decreased significantly as the concentration of citric acid increases. In addition to reducing solution pH, citric acid is known to crosslink anionic bio-polymers like cellulose and xanthan gum [55,56], as well as proteins and peptide compounds [58]. Thus, it is likely to do so with Spirulina biomass which is predominantly anionic in water [59,60] and contains both polysaccharides [61,62] and peptides [54]. Considering the crosslinking effects of citric acid, the decrease in shear-thickening behavior is thought to be in direct accordance with the amount of undenatured soft-body colloids remaining in each EOR fluid. If cross-linking occurs between the Spirulina biomass and citric acid, this would account for the volume increase of the “settled-phase” (see FIG. 50B) when centrifuging the well treatment compositions as recorded in the previous results of column 8 in Table 6 shown in FIG. 52. The citric acid could create insoluble components through protein denaturation, by crosslinking proteins into solid phases, or by acting as a flocculant that binds other solids together. The cross-linking effect of citric acid has been established in literature for gel technology [63-65], which reinforces the understanding that cross-linking the colloids will effectively reduce the amount left in suspension within the EOR fluid and thus result in reduced shear-thickening effects. As seen in the flow curve data, the citric acid-based well treatment compositions display the lowest viscosities at the low shear ranges, which means that fluid viscosity is likely to not a significant driving force in citric acid EOR performance.

Urea-based well treatment compositions displayed their shear-thickening region within the shear rate interval of 2,000-5,200 s⁻¹. Based on the trends, it can be seen that the addition of urea has reduced the intensity of the shear-thickening behavior significantly, but with minor reductions based on increasing the concentration of urea. These reductions are attributed to the known denaturation properties of urea [66]. However, it was observed that the urea-induced denaturation of the Spirulina biomass is a slow and gradual process, which would result in the less denaturation of the colloidal proteins in comparison to the more aggressive denaturants included in this study, thereby leaving more insoluble soft colloids (see FIG. 50C) comparable in quantity with the undenatured well treatment compositions. This explains why the previously discussed volume of “settled phase” in column 8 of Table 6 shown in FIG. 52 did not significantly change and the shear-thickening behavior was still visible in the flow curve experiments. The urea-based well treatment compositions displayed little increase regarding their low shear viscosity, which indicates that the lack of improved viscosity is likely to play a part in their low EOR performance.

The sodium hydroxide-based well treatment compositions experienced the most significant reduction in shear-thickening behavior, which only appears in the shear range of 2,000-4,000 s⁻¹. The reduction in shear-thickening intensity was more prominent as the concentration of sodium hydroxide increases, where the EOR fluid containing 2.33% sodium hydroxide did not exhibit any indication of a shear-thickening region over the entire interval of 0-20,000 s⁻¹. The sharp reduction in shear-thickening is attributed to the strong denaturant activity of sodium hydroxide [67], which was observed in this study to be a much faster process than denaturation induced by urea. Due to this more rapid denaturation process, it can be inferred that significantly more colloids were fully denatured and only a small amount of partially denatured colloids would remain, which explains the significant reduction in the volume of the “settled phase” in column 8 of Table 6 shown in FIG. 52. As shown in the flow curve data, the EOR fluid containing 2.33% of 1 M sodium hydroxide exhibited the highest viscosity at the low shear ranges, which indicates that fluid viscosity is highly likely to be a significant driving force in the performance of these well treatment compositions.

From the aforementioned results, it was determined that insoluble particle-particle interactions are more significant in increasing the intensity of shear-thickening than the insoluble particle interactions with either soluble and denatured proteins or crosslinked Spirulina gel structures. The observed discontinuous shear-thickening behavior of the Spirulina-derived soft-body colloids present in these well treatment compositions is consistent with the hydrodynamic lubrication theory, which agrees with the study of Kaldasch, et al. [68] and their discussion of particle volume fraction and its resulting particle-particle lubrication. In short, their interpretation states that increasing volume fraction of soft-body colloids will also increase the van der Waals attraction between individual colloids and result in colloid coagulation, which is increasingly more significant when the repulsion stabilization between the colloids is small. Furthermore, they explained that increasing this volume fraction results in relaxation times approaching infinity (elastic solid behavior), while decreasing this volume fraction results in the relaxation times approaching zero (viscous liquid behavior).

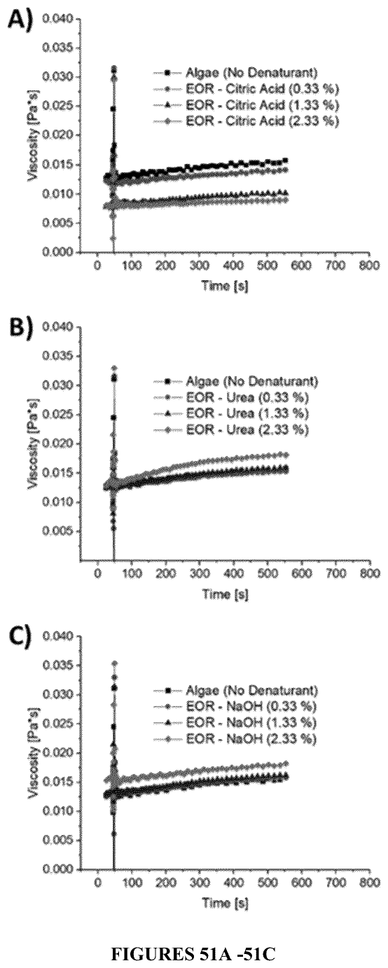

As shown in FIG. 51, all well treatment compositions were tested in a 3-interval-thixotropy test at a constant temperature of 20° C. The first interval is held at a constant shear to maintain the fluid structure, the second interval induces a high shear to break the fluid structure, and the third interval is held at a constant shear in order to measure the time needed for the fluid to recover its initial structure. The EOR fluid structures were stable at the low shear interval of 10 [symbol] 1, then experienced a breakdown of structure at the induced high shear interval of 200 s⁻¹. Additionally, it was observed that all solutions needed at most approximately 200 s to rebuild their structure to the initial state before experiencing the high-shear load. The sodium hydroxide-based well treatment compositions yield higher viscosities than the other two well treatment compositions at equivalent concentrations.

The citric acid-based well treatment compositions showed reduction in viscosity as the concentration of citric acid denaturant increased. The urea- and sodium hydroxide-based well treatment compositions displayed minor viscosity fluctuations as the concentration of 1 M denaturant increased from 0.33% to 1.33%. However, viscosity was shown to increase more significantly for the sodium hydroxide- and urea-based of well treatment compositions at 2.33% denaturant concentrations. All well treatment compositions display signs of slight viscosity increase as the steady shear continues over time.

Thixotropy results have shown that the structures of all well treatment compositions were able to sufficiently rebuild after experiencing a high shear load of 200 s⁻¹. Based on observations, all of the fluids seemed to recover their structures quickly and also slightly increase in viscosity. The rebuild time in these fluids seem to not be significantly driven by denaturant concentration. However, denaturant concentration may play a more significant role in thixotropy characterization of the solids after being separated from the liquid.

The total average tertiary recovery from undenatured Spirulina-based well treatment compositions was 17.21%. An average of 13.3% of oil was retrieved by the EOR agent based on the amount of oil initially in the sand pack. By centrifugation, 66.73% of the agglomeration can be recovered as oil, which may be added to the total yield of free oil. In these cases, the agglomeration tends to make up roughly 44% of the total output, increasing the total average yield to 17.21%. The total recovery observed is a combination of water flooding and EOR flooding, which was measured to be 39.39%. This is representative of an initial water flood phase to a 50% instantaneous water cut, followed by an injection of 2 pore volumes (PV) of the EOR agent. Compared to the previous study [9] on sand-pack flooding with denatured Spirulina-based well treatment compositions, the undenatured well treatment compositions displayed a reduced percentage of recovery.

Fluids containing 2.33% of each denaturant were also evaluated for their tertiary performance. The total tertiary recoveries for these fluids were 16.67%, 13.89%, and 21.43% for fluids containing citric acid, urea, and sodium hydroxide, respectively. Additionally, the total recovery percentages for these fluids are 43.03%, 38.89%, and 50.86%, respectively. Like the undenatured Spirulina well treatment compositions, these fluids also displayed a reduced percentage of recovery than the fluids containing both Spirulina and a denaturant.

The overall objective of this study is to investigate the active EOR mechanisms in fluids derived from denatured algal biomass which now can be analyzed using the p-value charts assembled in FIG. 52 One notable observation is that the EOR performance of the different well treatment compositions correlates with different properties which indicates that their EOR performances of the three denaturants are based on different mechanisms.

The EOR performance of the citric acid-based well treatment compositions have strong correlations with the settled-phase IFT and % of particulates, followed by concentration of citric acid and surface tension. Given the rigid, gel-like properties of these citric acid-based particulates and the p-value statistics, it is very likely that these components are a significant contributor to the EOR performance of the citric acid-based well treatment compositions. For the urea-based well treatment compositions, the most significant correlations with EOR performance are concentration of urea and settled-phase IFT, which are followed by surface tension and work of adhesion. For the sodium hydroxide-based well treatment compositions, the strongest correlations with EOR performance are concentration of sodium hydroxide, pH, and shear viscosity, followed by contact angle and work of adhesion.

Conceptually, it is well-accepted in the petrochemical industry that improved wettability, reduced IFT between oil and the aqueous phase, and increased viscosity are common driving mechanisms for improved EOR performance. As observed by the reduction in surface tension, IFT, and contact angle, all of the well treatment compositions exhibited good wettability with silicon dioxide surfaces. They also proved to be effective oil emulsifiers. Shear-thickening behavior was thought to be the rheological driving force behind the EOR performance of these novel fluids, which would improve the mobility ratio between the injection fluid and the hydro-carbon. Considering the rheological behavior and EOR performance of the sodium hydroxide-based well treatment compositions, the increase in viscosity seems to play a more dominant role in EOR performance than a shear-thickening rheology for this fluid.

The interfacial and rheological properties of Spirulina-based well treatment compositions were quantified to test their correlations with their oil recovery and to provide greater insight into the main driving mechanisms of the observed EOR effect. The liquid phase of all well treatment compositions yield reduced interfacial tension values with the hydraulic oil interface. From visual observation of the distinct color change and its gel-like characteristics, the settled-phase of the citric acid-based well treatment compositions is likely to be a new compound formed via reaction between citric acid and anionic Spirulina biomass which needs characterization in a future study.

The contact angle measurements indicated that increasing the concentration of denaturants of the well treatment compositions will improve wettability with sodium hydroxide displaying the best wettability enhancement. The work of adhesion of the well treatment compositions did not exceed the adhesion energy values of the tap water or brine, but the adhesion energy of well treatment compositions exceeds the adhesion energy of hydraulic oil. It can be concluded that the EOR effect is not largely driven by the work of adhesion, because the well treatment compositions recovered more oil than water while having lower works of adhesion to the substrate.

The rheology measurements determined that the well treatment compositions behave like viscoelastic solids where the storage modulus exceeds the loss modulus. The well treatment compositions containing sodium hydroxide displayed the highest viscosities at lower shear rates which is highly desired during polymer flooding applications in EOR. At higher shear rates (up to 20,000 s⁻¹), it is confirmed that most fluids can exhibit both shear-thickening and shear-thinning behavior. The intensity of the different non-Newtonian regimes is attributed to the presence of the soft-body colloids of the Spirulina biomass and their interactions with the denaturant. The discontinuous shear-thickening is thought to be a direct result of colloid volume and the particle-particle lubrication interactions, which is consistent with the hydrodynamic lubrication theory.

Additionally, it was found that the Spirulina-based well treatment compositions were able to achieve viscosity values (up to 0.0113 Pa·s at 100 s⁻¹), amplitude sweep LVER moduli values (between 1 and 8 Pa), and frequency sweep moduli values at 1 rad/s (between 1 and 20 Pa) that are comparable to the industry standard well treatment compositions of xanthan gum and HPAM.

The EOR performance of these fluids strongly correlates with the increased viscosity, reduced IFT, and increased wettability in the well treatment compositions. From the results, it is concluded that the performance of the different types of well treatment compositions is based on different mechanisms. Sodium hydroxide-based fluids seem to be driven by increased low-shear viscosity, reduced IFT, alkaline pH shift, and improved wettability. The citric acid-based fluids do not exhibit high viscosity values in the low shear range, which indicates that their EOR performance seems to be driven by reduced IFT, acidic pH shift, improved wettability, and increased fluid rigidity by the generation of crosslinks in the macro-molecular structure. In addition to lacking a significant pH shift, urea-based fluids also do not exhibit improved viscosity, which means that their modest EOR performance is likely driven by reduced IFT and improved wettability. While a shear-thickening rheology is often considered to be a mechanism for improving EOR, it is concluded that the shear-thickening effect is not a dominant mechanism for these fluids, especially since the highest performing EOR fluid (highest NaOH concentration) did not display shear thickening.

Table 7 shows a Certificate of Analysis for Spirulina plantesis.

TABLE 7
Certificate of Analysis for Spirulina plantesis

| ANALYSIS ITEM | SPECIFICATION | RESULTS | | METHOD |
|---|---|---|---|---|
| Appearance | Deep green fine powder | Complies | | Visual |
| Odor | Characteristic | Complies | | |
| Sieve analysis | 100% pass 100 mesh | Complies | | 100 mesh |
| Loss on Drying | ≤7.0% | 6.8% | | Sreen |
| | | | | 105° C./ |
| | | | | 3 hrs |
| Ashes | ≤9% | 7.2% | | |
| Chlorophyll | ≥1.0% | 1.3% | | |
| Carotenoid (g/kg) | ≥2.0 | 2.0 | | CP2000 |
| Pb (ppm) | <2.0 | <1.2 | | CP2000 |
| Cd (ppm) | ≤0.2 | ≤0.1 | | |
| Hg (ppm) | ≤0.05 | <0.05 | | |
| As (ppm) | <0.5 | Complies | | AAS |
| Total Plate Count | <30000/g | 8000 | cfu/g | |
| Yeast & Mold | <90/g | 30 | cfu/g | |

| | | | |
|---|---|---|---|
| E.Coil | Negative | Complies | |
| Salmonella | Negative | Complies | |
| Crude protein | ≥60% | 62.2% | CP2000 |

The Table 8 was obtained using the same protocols as the other EOR, but instead Chlorella vulgaris was used instead of Spirulina, and crude oil was used instead of hydraulic fluid.

TABLE 8
Well Treatment Compositions
including Chlorella vulgaris

| | | EOR | total |
|---|---|---|---|
| | NaOH | oil | EOR |
| Algae | solution | recovery | recovery |
| added (g) | added (mL) | % | % |
| 90 | 17.5 | 36.14% | 60.74% |
| 90 | 20 | 41.21% | 61.37% |
| 90 | 22.5 | 45.21% | 65.44% |
| 90 | 25 | 54.05% | 71.67% |
| 50 | 15 | 18.18% | 46.76% |
| 70 | 15 | 26.19% | 54.41% |
| 90 | 15 | 26.04% | 52.09% |
| 110 | 15 | 27.27% | 55.00% |

One of ordinary skill in the art will readily appreciate that alternative but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits may be the values from the examples. Certain lower limits, upper limits and ranges appear in at least one claims below. All numerical values are “about” or “approximately” the indicated value, and consider experimental error and variations that would be expected by a person having ordinary skill in the art.

### REFERENCES

IEA—International Energy Agency n.d. https://www.iea.org/ (accessed Dec. 28, 2021).
Whatever happened to enhanced oil recovery?—Analysis—IEA n.d. https://www.iea.org/commentaries/whatever-happened-to-enhanced-oil-recovery (accessed Dec. 28, 2021).
Strom E T, Paul J M, Phelps C H, Sampath K. New biopolymer for high-temperature profile control. Part 1. Laboratory testing. SPE Resery Eng (Society Pet Eng 1991. https://doi.org/10.2118/19633-PA.
Blokker N C M. Analysis of Alginate-like Exopolysaccharides for the Application in Enhanced Oil Recovery 2014.
Olajire A A. Review of ASP EOR (alkaline surfactant polymer enhanced oil recovery) technology in the petroleum industry: Prospects and challenges. Energy 2014; 77:963-82. https://doi.org/10.1016/j.energy.2014.09.005.
Schexnayder P, Baudoin N, Chirdon W M. Enhanced oil recovery from denatured algal biomass: Synergy between conventional and emergent fuels. Fuel 2020: 119533. https://doi.org/10.1016/j.fuel.2020.119533.
Nikolova C, Gutierrez T. Use of microorganisms in the recovery of oil from recalcitrant oil reservoirs: current state of knowledge, technological advances and future perspectives. Front Microbiol 2020:10. https://doi.org/10.3389/fmicb.2019.02996.
Yernazarova A, Kayirmanova G, Zhubanova A B and A. Microbial enhanced oil recovery. In: Romero-Zeron L, editor., Rijeka: IntechOpen; 2016, p. Ch. 5. https://doi.org/10.5772/64805.
Haq B. The role of microbial products in green enhanced oil recovery: acetone and butanone. Polymers (Basel) 2021:13. https://doi.org/10.3390/polym13121946.
Wang J, Tang X, Li J, Dai L, Deng G, Zhang Z. Sawdust biomass promotes aquathermolysis of extra-heavy oil. Fuel Process Technol 2022; 238:107522. https://doi.org/10.1016/j.fuproc.2022.107522.
Sugai Y, Komatsu K, Sasaki K. Estimation of IFT reduction by a biomass material and potential of its utilization for EOR. SPE/IATMI Asia Pacific Oil Gas Conf Exhib 2015: SPE-176490-MS. https://doi.org/10.2118/176490-MS.
Armstrong R T, Wildenschild D. Investigating the pore-scale mechanisms of microbial enhanced oil recovery. J Pet Sci Eng 2012; 94-95:155-64. https://doi.org/10.1016/j.petrol.2012.06.031.
Pandey R, Krishnamurthy B, Singh H P, Batish D R. Evaluation of a glycolipopepetide biosurfactant from Aeromonas hydrophila RP1 for bioremediation and enhanced oil recovery. J Clean Prod 2022:345. https://doi.org/10.1016/j.jclepro.2022.131098.
Al-Ghamdi A, Haq B, Al-Shehri D, Muhammed N S, Mahmoud M. Surfactant formulation for green enhanced oil recovery. Energy Rep 2022; 8:7800-13. https://doi.org/10.1016/j.egyr.2022.05.293.
Obuebite A A, Victor-Oji C O, Eke W I. Laboratory evaluation of red onion skin extract and its derivative as biomass-based enhanced oil recovery agents. Sci Afr 2023; 19:e01460. https://doi.org/10.1016/j.sciaf.2022.e01460.
Mtui H I, Liu F-H, Wang W, Lang J-Q, Yang S-Z, Mu B-Z. Renewable non-edible oils derived long chain (C24.1) bio-based zwitterionic surfactant with ultralow interfacial tension between crude oil and formation brine. Green Chem Eng 2022. https://doi.org/10.1016/j.gce.2022.08.001.
Tajik A, Farhadian A, Khelkhal M A, Rezaeisadat M, Petrov S M, Eskin A A, et al. Sunflower oil as renewable biomass source to develop highly effective oil-soluble catalysts for in-situ combustion of heavy oil. Chem Eng J 2023; 453:139813. https://doi.org/10.1016/j.cej.2022.139813.
Sharma K, Toor S S, Branda\tilde{ }o J, Pedersen T H, Rosendahl L A. Optimized conversion of waste cooking oil into ecofriendly bio-based polymeric surfactant—A solution for enhanced oil recovery and green fuel compatibility. J Clean Prod 2021; 294: 126214. https://doi.org/10.1016/j.clepro.2021.126214.
Priyanto S, Kusworo T D, Sayyidah, Pramudono B, Untoro E, Ratu P. Characterization and purification of surfactant sodium ligno sulfonate (SLS) from biomass waste in the application of enhanced oil recovery (EOR). J Phys Conf Ser 2019; 1295(1):012015. https://doi.org/10.1088/1742-6596/1295/1/012015.
Chirdon W M, O'Brien W J, Robertson R E. Adsorption of catechol and comparative solutes on hydroxyapatite. J Biomed Mater Res—Part B Appl Biomater 2003; 66: 532-8. https://doi.org/10.1002/jbm.b.10041.
Sharma J, Kapley A, Sundar D, Srivastava P. Characterization of a potent biosurfactant produced from Franconibacter sp. IITDAS19 and its application in enhanced oil recovery. Colloids Surf B Biointerfaces 2022; 214:112453. https://doi.org/10.1016/j.colsurfb.2022.112453.
Li J, Jiang Z, Wang Y, Zheng J, Huang G. Stability, seepage and displacement characteristics of heterogeneous branched-preformed particle gels for enhanced oil recovery. RSC Adv 2018; 8:4881-9. https://doi.org/10.1039/c7ra13152f.
Nwufoh P, Hu Z, Wen D, Wang M. Nanoparticle assisted EOR during sand-pack flooding: electrical tomography to assess flow dynamics and oil recovery. Sensors (Basel) 2019:19. https://doi.org/10.3390/s19143036.
Pegram L M, Record M T. Hofmeister salt effects on surface tension arise from partitioning of anions and cations between bulk water and the air-water interface. J Phys Chem B 2007; 111:5411-7. https://doi.org/10.1021/jp070245z.
Virga E, Spruijt E, de Vos W M, Biesheuvel P M. Wettability of amphoteric surfaces: the effect of pH and ionic strength on surface ionization and wetting. Langmuir 2018; 34:15174-80. https://doi.org/10.1021/acs.langmuir.8b02875.
Mwangi P, Brady P V, Radonjic M, Thyne G. The effect of organic acids on wettability of sandstone and carbonate rocks. J Pet Sci Eng 2018; 165:428-35. https://doi.org/10.1016/j.petrol.2018.01.033.
Bertsch P, Bo\ddot{ }cker L, Mathys A, Fischer P. Proteins from microalgae for the stabilization of fluid interfaces, emulsions, and foams. Trends Food Sci Technol 2021; 108:326-42. https://doi.org/10.1016/j.tifs.2020.12.014.
Rosen M, Wang H, Shen P, Zhu Y. Ultralow interfacial tension for enhanced oil recovery at very low surfactant concentrations. Langmuir 2005; 21:3749-56. https://doi.org/10.1021/la0400959.
Liu Q, Liu S, Luo D, Peng B. Ultra-low interfacial tension foam system for enhanced oil recovery. Appl Sci 2019:9. https://doi.org/10.3390/app9102155.

Xu L, Xu G, Liu T, Chen Y, Gong H. The comparison of rheological properties of aqueous welan gum and xanthan gum solutions. Carbohydr Polym 2013; 92: 516-22. https://doi.org/10.1016/j.carbpol.2012.09.082.
Gansbiller M, Schmid J, Sieber V. In-depth rheological characterization of genetically modified xanthan-variants. Carbohydr Polym 2019; 213:236-46. https://doi.org/10.1016/j.carbpol.2019.02.055.
Aliabadian E, Sadeghi S, Rezvani Moghaddam A, Maini B, Chen Z, Sundararaj U. Application of graphene oxide nanosheets and HPAM aqueous dispersion for improving heavy oil recovery: Effect of localized functionalization. Fuel 2020; 265: 116918. https://doi.org/10.1016/j.fuel.2019.116918.
Corredor L M, Aliabadian E, Husein M, Chen Z, Maini B, Sundararaj U. Heavy oil recovery by surface modified silica nanoparticle/HPAM nanofluids. Fuel 2019; 252: 622-34. https://doi.org/10.1016/j.fuel.2019.04.145.
Alquraishi A A, Alsewailem F D. Xanthan and guar polymer solutions for water shut off in high salinity reservoirs. Carbohydr Polym 2012; 88:859-63. https://doi.org/10.1016/j.carbpol.2012.01.022.
Xu L, Dong M, Gong H, Sun M, Li Y. Effects of inorganic cations on the rheology of aqueous welan, xanthan, gellan solutions and their mixtures. Carbohydr Polym 2015; 121:147-54. https://doi.org/10.1016/j.carbpol.2014.12.030.
Ogunkunle T F, Oni B A, Afolabi R O, Fadairo A S, Ojo T, Adesina O. Comparative analysis of the performance of hydrophobically associating polymer, xanthan and guar gum as mobility control agent, in enhanced oil recovery application. J King Saud Univ—Eng Sci 2022. https://doi.org/10.1016/j.jksues.2022.01.003.
Lizcano Nino J C, De Sousa F V H, Moreno Rosangela B Z L. Less-concentrated hpam solutions as a polymer retention reduction method in ceor. Fuentes El Revent Energ 2020; 18:75-92. https://doi.org/10.18273/REVFUE.V17N1-2020008.
Al-Shakry B, Skauge T, Shiran B S, Skauge A. Impact of mechanical degradation on polymer injectivity in porous media. Polymers (Basel) 2018; 10:1-23. https://doi.org/10.3390/polym10070742.
Lotfollahi M, Farajzadeh R, Delshad M, Al-Abri K, Wassing B M, Mjeni R, et al. Mechanistic simulation of polymer injectivity in field tests. Soc Pet Eng—SPE Asia Pacific Enhanc Oil Recover Conf EORC 2015 2015:1079-106. https://doi.org/10.2118/174665-ms.
Azad MS, Patel V, Shah N, Sharma T, Trivedi J J. Extensional rheological measurements of surfactant-polymer mixtures. ACS Omega 2020; 5:30787-98. https://doi.org/10.1021/acsomega.0c00481.
Ferreira V, Moreno R. Polyacrylamide adsorption and readsorption in sandstone porous media. SPE J 2019:25. https://doi.org/10.2118/199352-PA.
Maaref S, Kantzas A, Bryant S. Investigating sweep efficiency improvement in oil-wet porous media by the application of functionalized nanoparticles. Fuel 2020; 267:117263. https://doi.org/10.1016/j.fuel.2020.117263.
Wagner N J, Brady J F. Shear thickening in colloidal dispersions. Phys Today 2009; 62:27-32. https://doi.org/10.1063/1.3248476.
Vern\grave{ }es L, Abert-Vian M, El Maetaoui M, Tao Y, Bornard I, Chemat F. Application of ultrasound for green extraction of proteins from Spirulina. Mechanism, optimization, modeling, and industrial prospects. Ultrason Sonochem 2019; 54: 48-60. https://doi.org/10.1016/j.ultsonch.2019.02.016.
Jaspe J, Hagen S J. Do protein molecules unfold in a simple shear flow? Biophys J 2006; 91:3415-24. https://doi.org/10.1529/biophysj.106.089367.
Hongsthong A, Sirijuntarut M, Prommeenate P, Thammathorn S, Bunnag B, Cheevadhanarak S, et al. Revealing differentially expressed proteins in two morphological forms of Spirulina platensis by proteomic analysis. Mol Biotechnol 2007; 36(2):123-30. https://doi.org/10.1007/s12033-007-0013-5.
Raucci M G, Alvarez-Perez M A, Demitri C, Giugliano D, De Benedictis V, Sannino A, et al. Effect of citric acid crosslinking cellulose-based hydrogels on osteogenic differentiation. J Biomed Mater Res Part A 2015; 103:2045-56. https://doi.org/10.1002/jbm.a.35343.
Li Y, Zhang D, Ash J, Jia X, Leone A, Templeton A. Mechanism and impact of excipient incompatibility: cross-linking of xanthan gum in pediatric powder-for-suspension formulations. J Pharm Sci 2019; 108:3609-15. https://doi.org/10.1016/j.xphs.2019.07.005.
Li T, Wang C, Li T, Ma L, Sun D, Hou J, et al. Surface hydrophobicity and functional properties of citric acid cross-linked whey protein isolate: The impact of pH and concentration of citric acid. Molecules 2018; 23(9):2383. https://doi.org/10.3390/molecules23092383.
Nourbakhsh H, Emam-Djomeh Z, Madadlou A, Mousavi M E, Moosavi-Movahedi A A, Gunasekaran S. Antioxidant peptidic particles for delivery of gallic acid. J Food Process Presery 2017; 41:e12767. https://doi.org/https://doi.org/10.1111/jfpp.12767.
Michalak I, Mironiuk M, Godlewska K, Trynda J, Marycz K. Arthrospira (Spirulina) platensis: An effective biosorbent for nutrients. Process Biochem 2020; 88:129-37. https://doi.org/10.1016/j.procbio.2019.10.004.
Dmytryk A, Saeid A, Chojnacka K. Biosorption of microelements by Spirulina: Towards technology of mineral feed supplements. Sci World J 2014; 2014:1-15. https://doi.org/10.1155/2014/356328.
Wang B, Cai T, Liu Q, Whitney J, Du M, Ma Q, et al. Preparation and evaluation of Spirulina polysaccharide nanoemulsions. Int J Mol Med 2018. https://doi.org/10.3892/ijmm.2018.3717.
Li T T, Huang Z R, Jia R B, Lv X C, Zhao C, Liu B. Spirulina platensis polysaccharides attenuate lipid and carbohydrate metabolism disorder in high-sucrose and high-fat diet-fed rats in association with intestinal microbiota. Food Res Int 2021; 147: 110530. https://doi.org/10.1016/j.foodres.2021.110530.
Ghorpade V S, Yadav A V, Dias R J, Mali K K, Pargaonkar S S, Shinde P V, et al. Citric acid crosslinked carboxymethylcellulose-poly(ethylene glycol) hydrogel films for delivery of poorly soluble drugs. Int J Biol Macromol 2018; 118:783-91. https://doi.org/10.1016/j.ijbiomac.2018.06.142.
Kanafi N M, Rahman N A, Rosdi N H. Citric acid cross-linking of highly porous carboxymethyl cellulose/poly(ethylene oxide) composite hydrogel films for controlled release applications. Mater Today Proc 2019; 7:721-31. https://doi.org/10.1016/j.matpr.2018.12.067.
Saito H, Taguchi T, Kobayashi H, Kataoka K, Tanaka J, Murabayashi S, et al. Physicochemical properties of gelatin gels prepared using citric acid derivative. Mater Sci Eng C 2004; 24:781-5. https://doi.org/10.1016/j.msec.2004.08.039.
Rossky P J. Protein denaturation by urea: slash and bond. Proc Natl Acad Sci U S A 2008; 105:16825-6. https://doi.org/10.1073/pnas.0809224105.
Mohammadi Z, Dummer P M H. Properties and applications of calcium hydroxide in endodontics and dental traumatology. Int Endod J 2011; 44:697-730. https://doi.org/10.1111/j.1365-2591.2011.01886.x.
Kaldasch J, Senge B, Laven J. Shear thickening in concentrated soft sphere colloidal suspensions: A shear induced phase transition. J Thermodyn 2015; 2015: 153854. https://doi.org/10.1155/2015/153854.

What is claimed is:

1. A well treatment composition, wherein the well treatment composition comprises:
denatured algae, wherein the denatured algae have a protein content from about 20.0 wt % to about 90.0 wt %, and where the denatured algae have a lipid content from about 0.1 wt % to about 30.0 wt %;
one or more carrier fluids; and
one or more additives.

2. A well treatment composition of claim 1, wherein the one or more carrier fluids are present from about 91 wt % to about 99.9 wt %.

3. The well treatment composition of claim 1, wherein the denatured algae comprise Spirulina plantesis.

4. The well treatment composition of claim 1, wherein the denatured algae comprise Chlorella vulgaris.

5. The well treatment composition of claim 1, wherein the well treatment composition has a viscosity from about 0.005 Pa·s to about 0.015 Pa·s over a shear rate range of about 0 to 20,000 s⁻¹.

6. A well treatment composition of claim 2, wherein one or more additives are present from about 0.1 wt % to about 20 wt %.

7. A method of making a well treatment composition, wherein the method of making the well treatment composition comprises:
contacting one or more algae with one or more denaturants to make a denatured algae, wherein the denatured algae have a protein content from about 20.0 wt % to about 90.0 wt %, and where the denatured algae have a lipid content from about 0.1 wt % to about 30.0 wt %; and
contacting the denatured algae with one or more carrier fluids to make a well treatment composition.

8. The method of making a well treatment composition of claim 7, wherein the one or more carrier fluids are present from about 91.0 wt % to about 99.9 wt %.

9. The method of making a well treatment composition of claim 7, wherein the denatured algae comprise Spirulina plantesis.

10. The method of making a well treatment composition of claim 7, wherein the denatured algae comprise Chlorella vulgaris.

11. The method of making a well treatment composition of claim 7, wherein the well treatment composition has a viscosity from about 0.005 Pa·s to about 0.015 Pa·s over a shear rate range of about 0 to 20,000 s⁻¹.

12. The method of making a well treatment composition of claim 7, wherein one or more denaturants is selected from a list consisting of: sodium hydroxide, urea, and citric acid.

13. A well treatment composition of claim 8, wherein the one or more additives are present from about 0.1 wt % to about 20.0 wt %.

14. A method of treating a well or subterranean formation, the method comprising:
injecting a well treatment composition into a wellbore, wherein the well treatment composition comprises:
one or more denatured algae, wherein the denatured algae have a protein content from about 20.0 wt % to about 90.0 wt %, and where the denatured algae have a lipid content from about 0.1 wt % to about 30.0 wt %;
one or more carrier fluids; and
one or more additives.

15. The method of treating a well or subterranean formation of claim 14, wherein the one or more carrier fluids, wherein the one or more carrier fluids are present from about 91.0 wt % to about 99.0 wt %.

16. The well treatment composition of claim 14, wherein the denatured algae comprise Spirulina plantesis.

17. The well treatment composition of claim 14, wherein the denatured algae comprise Chlorella vulgaris.

18. The well treatment composition of claim 14, wherein the well treatment composition has a viscosity from about 0.005 Pa·s to about 0.015 Pa·s over a shear rate range of about 0 to 20,000 s⁻¹.

19. The well treatment composition of claim 14, wherein the one or more denatured algae has a component arising after denaturation with a molecular weight from about 225,000 g/mole to about 275,000.

20. A well treatment composition of claim 15, wherein the one or more additives are present from about 0.1 wt % to about 20.0 wt %.
